(19) United States
(12) Patent Application Publication
(10) Pub. No.: US 2022/0241278 A1
(43) Pub. Date: Aug. 4, 2022

(54) INHALABLE IMATINIB FORMULATION

(71) Applicant: InCarda Therapeutics, Inc., Newark, CA (US)

(72) Inventors: Carlos SCHULER, Kensington, CA (US); Michael Laird HURREY, San Ramon, CA (US); Grace E. COLON, Los Gatos, CA (US); Brian WIEST, Castro Valley, CA (US)

(21) Appl. No.: 17/719,042

(22) Filed: Apr. 12, 2022

Related U.S. Application Data
(63) Continuation of application No. PCT/US2022/011448, filed on Jan. 6, 2022.
(60) Provisional application No. 63/134,336, filed on Jan. 6, 2021.
(60) Provisional application No. 63/170,246, filed on Apr. 2, 2021.

(51) Int. Cl. A61K 31/506; A61K 47/40; A61K 9/00

(52) U.S. Cl. CPC A61K 31/506; A61K 9/007; A61K 47/40

(57) ABSTRACT

Pharmaceutical compositions comprising imatinib or a derivative thereof for treatment of a pulmonary disease via inhalation. Methods of treating a pulmonary disease include administering by inhalation an effective amount of imatinib or a derivative thereof to a patient in need thereof. In aspects, the pharmaceutical composition provided herein comprises an aqueous solution or suspension of imatinib or a derivative thereof that is formulated for inhalatory administration.

### CROSS-REFERENCE

This application is a continuation of International Patent Application No. PCT/US2022/011448, filed Jan. 6, 2022, which claims the benefits of U.S. Provisional Patent Application No. 63/134,336, filed Jan. 6, 2021, and U.S. Provisional Patent Application No. 63/170,246, filed Apr. 2, 2021, each of which is incorporated herein by reference in its entirety for all purposes.

### BACKGROUND

Imatinib is a small molecule kinase inhibitor that can inhibit the Bcr-Abl tyrosine kinase, the constitutive abnormal tyrosine kinase created by the Philadelphia chromosome abnormality in chronic myeloid leukemia (CML). It can be used to treat certain types of cancer. It is currently marketed by Novartis as Gleevec (USA) or Glivec (Europe/Australia) as its mesylate salt, imatinib mesilate (INN). It can inhibit proliferation and induce apoptosis in Bcr-Abl positive cell lines as well as fresh leukemic cells from Philadelphia chromosome positive chronic myeloid leukemia. Imatinib can also inhibit the receptor tyrosine kinases for platelet derived growth factor (PDGF) and stem cell factor (SCF). There are also reports that injection (subcutaneous or intraperitoneal) or oral delivery of imatinib mesylate can have therapeutic effects on pulmonary arterial hypertension in animal models and patients enrolled in clinical trials.

### SUMMARY

In some aspects, disclosed herein is a composition, comprising an aqueous solution or suspension that comprises: (1) imatinib or a derivative thereof, (2) a solubility enhancer, and (3) a pH buffer, wherein the aqueous solution or suspension: (a) has a concentration of the imatinib or derivative thereof of from 20 to 500 mg/mL; (b) has a viscosity of at most 10 centipoise; and (c) has a pH of 3 to 8.

In some embodiments of the composition, the solubility enhancer is selected from the group consisting of: cyclodextrins, lipids, co-solvents, organic acids, and sufactants. In some cases of the composition, the solubility enhancer comprises a cyclodextrin. In some cases of the composition, the aqueous solution or suspension has the cyclodextrin at a concentration of from about 1% (w/v) to about 80% (w/v). In some cases of the composition, the solubility enhancer comprises a lipid or a fatty acid. In some cases of the composition, the lipid or fatty acid is selected from the group consisting of: polyethoxylated castor oil, phospholipids, glycolipids, ganglioside GM1, sphingomyelin, phosphatidic acid, cardiolipin; lipids bearing polymer chains such as polyethylene glycol (PEG), chitin, hyaluronic acid, and polyvinylpyrrolidone; lipids bearing sulfonated monosaccharides, lipid-bearing sulfonated disaccharides, lipid bearing sulfonated polysaccharides; fatty acids such as palmitic acid, stearic acid, and oleic acid; cholesterol, cholesterol esters, and cholesterol hemisuccinate. In some cases of the composition, the solubility enhancer comprises a co-solvent. In some cases of the composition, the co-solvent comprises glycerol or ethanol. In some cases of the composition, the solubility enhancer comprises an organic acid. In some cases of the composition, the organic acid is selected from the group consisting of: acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric acid, tannic acid, L(+)-tartaric acid, taurocholic acid, and thiodipropionic acid. In some cases, the solubility enhancer comprises a surfactant. In some cases, the surfactant comprises Tween, sodium lauryl sulfate (SLS), or dipalmitoylphosphatidylcholine (DPPC).

In some aspects, disclosed herein is a composition, comprising an aqueous solution or suspension that comprises imatinib or a derivative thereof and cyclodextrin, wherein the aqueous solution or suspension has the cyclodextrin at a concentration of from about 1% (w/v) to about 80% (w/v).

In some cases of the composition, the cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins. In some cases of the composition, the cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin. In some cases of the composition, the cyclodextrin comprises an anionic cyclodextrin.

In some aspects, disclosed herein is a composition, comprising an aqueous solution or suspension that comprises imatinib or a derivative thereof and cyclodextrin, wherein the cyclodextrin comprises an anionic cyclodextrin.

In some cases of the composition, the aqueous solution or suspension further comprises a pH buffer. In some cases of the composition, the pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer. In some cases of the composition, the pH buffer comprises a phosphate buffer.

In some aspects, disclosed herein is a composition, comprising an aqueous solution or suspension that comprises imatinib or a derivative thereof, cyclodextrin, a pH buffer, and a surfactant.

In some cases of the composition, the aqueous solution or suspension comprises a salt of the cyclodextrin. In some cases of the composition, salt of said cyclodextrin is a salt selected from the group consisting of: sodium salt, calcium salt, magnesium salt, iron salt, chromium salt, copper salt, zinc salt, lysine salt, arginine salt, and histidine salt. In some cases of the composition, the cyclodextrin comprises sulfobutylether-β-cyclodextrin. In some cases of the composition, the cyclodextrin comprises hydroxypropyl-β-cyclodextrin. In some cases of the composition, the aqueous solution or suspension comprises sulfobutylether-β-cyclodextrin sodium.

In some cases of the composition, the aqueous solution or suspension further comprises a surfactant. In some cases of the composition, the surfactant comprises Tween, sodium lauryl sulfate (SLS), or dipalmitoylphosphatidylcholine (DPPC).

In some cases of the composition, the aqueous solution or suspension has a viscosity of at most 10 centipoise. In some cases of the composition, the aqueous solution or suspension has a viscosity of at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise. In some cases of the composition, the aqueous solution or suspension has a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.

In some cases of the composition, the aqueous solution or suspension has from 20 to 500 mg/mL of the imatinib or derivative thereof. In some cases of the composition, the aqueous solution or suspension has from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of the imatinib or derivative thereof. In some cases of the composition, the aqueous solution or suspension has about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of the imatinib or derivative thereof. In some cases of the composition, the aqueous solution or suspension has about 80 mg/mL of the imatinib or derivative thereof.

In some cases of the composition, the aqueous solution or suspension has a pH of 3 to 8. In some cases of the composition, the pH of the aqueous solution or suspension is from 3 to 6, from 4 to 6, from 4.5 to 5.5, from 5 to 6, from 4 to 7, from 5 to 7, or from 6 to 7. In some cases of the composition, the pH of the aqueous solution or suspension is about 4.5, about 4.7, about 4.8, about 4.9, about 5.0, about 5.1, about 5.2, about 5.4, about 5.5, or about 5.6. In some cases of the composition, the pH of the aqueous solution or suspension is from 7 to 8. In some cases of the composition, the pH of the aqueous solution or suspension is about 7.0, about 7.2, about 7.4, about 7.6, about 7.8, or about 8.0.

In some cases of the composition, the aqueous solution or suspension has the cyclodextrin at a concentration of from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v). In some cases of the composition, the aqueous solution or suspension has the cyclodextrin at a concentration of from 5% (w/v) to 40% (w/v). In some cases of the composition, the aqueous solution or suspension has the cyclodextrin at a concentration of from 10% (w/v) to 20% (w/v). In some cases of the composition, the aqueous solution or suspension has the cyclodextrin at a concentration of from 25% (w/v) to 40% (w/v). In some cases of the composition, the aqueous solution or suspension has the cyclodextrin at a concentration of about 10% (w/v), about 12% (w/v), about 14% (w/v), about 15% (w/v), about 16% (w/v), about 18% (w/v), or about 20% (w/v). In some cases of the composition, the aqueous solution or suspension has the cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

In some cases of the composition, the composition comprises the aqueous solution. In some cases of the composition, the solubility of the imatinib or derivative thereof in the aqueous solution is negatively correlated with the pH of the aqueous solution. In some cases of the composition, the solubility of the imatinib or derivative thereof in the aqueous solution is positively correlated with concentration of the cyclodextrin in the aqueous solution.

In some cases, the composition comprises the aqueous suspension.

In some cases of the composition, the composition comprises less than 1 mg/mL, less than 0.5 mg/mL, less than 0.1 mg/mL, less than 0.05 mg/mL, less than 0.01 mg/mL, less than 0.005 mg/mL, less than 0.001 mg/mL, or less than 0.0001 mg/mL imatinib mesylate. In some cases of the composition, the composition does not comprise imatinib mesylate. In some cases of the composition, the imatinib or derivative thereof comprises imatinib free base. In some cases of the composition, the imatinib or derivative thereof is imatinib free base. In some cases of the composition, the composition comprises a salt of the imatinib or derivative thereof selected from the group consisting of: acetate salt, formate salt, citrate salt, phosphate salt, maleate salt, fumarate salt, tartrate salt, malonate salt, lactic salt, and succinate salt.

In some aspects, disclosed herein is a pharmaceutical composition, comprising the composition disclosed herein. In some cases, the pharmaceutical composition is formulated for inhalatory administration. In some cases of the pharmaceutical composition, the aqueous solution further comprises a pharmaceutically acceptable excipient. In some cases, the pharmaceutically acceptable excipient comprises a surfactant. In some cases of the pharmaceutical composition, the surfactant comprises Tween, sodium lauryl sulfate (SLS), or dipalmitoylphosphatidylcholine (DPPC). In some cases, the pharmaceutically acceptable excipient comprises a lipid. In some cases of the pharmaceutical composition, the lipid comprises a polymeric lipid, a sulfonated poly saccharide, or a fatty acid. In some cases of the pharmaceutical composition, the lipid comprises a polymeric lipid, a sulfonated poly saccharide, or a fatty acid. In some cases of the pharmaceutical composition, the pharmaceutical composition is organoleptically tolerated when inhaled by a human subject. In some cases of the pharmaceutical composition, pharmaceutical composition does not induce cough reflex when inhaled by a human subject. In some cases of the pharmaceutical composition, the pharmaceutical composition is not or minimally irritative to mouth or throat when inhaled by a human subject.

In some aspects, disclosed herein is a pharmaceutical composition, comprising an aqueous solution that comprises cyclodextrin and a therapeutically effective amount of imatinib or a derivative thereof, wherein the aqueous solution is formulated for inhalatory administration.

In some cases of the pharmaceutical composition, the aqueous solution has a viscosity of at most 10 centipoise. In some cases of the pharmaceutical composition, the aqueous solution has a viscosity of at most 2.5 centipoise.

In some cases of the pharmaceutical composition, the aqueous solution has from 20 to 500 mg/mL of the imatinib or derivative thereof. In some cases of the pharmaceutical composition, the aqueous solution has from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of the imatinib or derivative thereof. In some cases of the pharmaceutical composition, the aqueous solution has about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of the imatinib or derivative thereof. In some cases of the pharmaceutical composition, the aqueous solution has about 80 mg/mL of the imatinib or derivative thereof.

In some cases of the pharmaceutical composition, the aqueous solution has a pH of 3 to 8. In some cases of the pharmaceutical composition, the pH of the aqueous solution is from 3 to 6, from 4 to 6, from 4.5 to 5.5, from 5 to 6, from 4 to 7, from 5 to 7, or from 6 to 7. In some cases of the pharmaceutical composition, the pH of the aqueous solution is about 4.5, about 4.7, about 4.8, about 4.9, about 5.0, about 5.1, about 5.2, about 5.4, about 5.5, or about 5.6. In some cases of the pharmaceutical composition, the pH of the aqueous solution or suspension is from 7 to 8. In some cases of the pharmaceutical composition, the pH of the aqueous solution is about 7.0, about 7.2, about 7.4, about 7.6, about 7.8, or about 8.0. In some cases of the pharmaceutical composition, the aqueous solution further comprises a pH buffer. In some cases of the pharmaceutical composition, the pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

In some cases of the pharmaceutical composition, the cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins. In some cases of the pharmaceutical composition, the cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin. In some cases of the pharmaceutical composition, the cyclodextrin comprises an anionic cyclodextrin. In some cases of the pharmaceutical composition, the cyclodextrin comprises sulfobutylether-β-cyclodextrin. In some cases of the pharmaceutical composition, the cyclodextrin comprises hydroxypropyl-β-cyclodextrin. In some cases of the pharmaceutical composition, the aqueous solution comprises a salt of the cyclodextrin. In some cases of the pharmaceutical composition, salt of said cyclodextrin is a salt selected from the group consisting of: sodium salt, calcium salt, magnesium salt, iron salt, chromium salt, copper salt, zinc salt, lysine salt, arginine salt, and histidine salt. In some cases of the pharmaceutical composition, the aqueous solution comprises sulfobutylether-β-cyclodextrin sodium

In some cases of the pharmaceutical composition, the aqueous solution has the cyclodextrin at a concentration of from about 1% (w/v) to about 80% (w/v), from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v). In some cases of the pharmaceutical composition, the aqueous solution has the cyclodextrin at a concentration of from 5% (w/v) to 40% (w/v). In some cases of the pharmaceutical composition, the aqueous solution has the cyclodextrin at a concentration of from 10% (w/v) to 20% (w/v). In some cases of the pharmaceutical composition, the aqueous solution has the cyclodextrin at a concentration of from 25% (w/v) to 40% (w/v). In some cases of the pharmaceutical composition, the aqueous solution has the cyclodextrin at a concentration of about 10% (w/v), about 12% (w/v), about 14% (w/v), about 15% (w/v), about 16% (w/v), about 18% (w/v), or about 20% (w/v). In some cases of the pharmaceutical composition, the aqueous solution has the cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

In some cases, the pharmaceutical composition is organoleptically tolerated when inhaled by a human subject. In some cases, the pharmaceutical composition does not induce cough reflex when inhaled by a human subject. In some cases, the pharmaceutical composition is not or minimally irritative to mouth or throat when inhaled by a human subject.

In some cases, the pharmaceutical composition comprises less than 1 mg/mL, less than 0.5 mg/mL, less than 0.1 mg/mL, less than 0.005 mg/mL, less than 0.001 mg/mL, or less than 0.0001 mg/mL imatinib mesylate. In some cases, the pharmaceutical composition does not comprise imatinib mesylate. In some cases of the pharmaceutical composition, the imatinib or derivative thereof comprises imatinib free base. In some cases of the pharmaceutical composition, the imatinib or derivative thereof is imatinib free base.

In some cases, the pharmaceutical composition comprises a salt of the imatinib or derivative thereof selected from the group consisting of: acetate salt, formate salt, citrate salt, phosphate salt, maleate salt, fumarate salt, tartrate salt, malonate salt, lactic salt, and succinate salt.

In some cases of the pharmaceutical composition, the aqueous solution further comprises a pharmaceutically acceptable excipient. In some cases of the pharmaceutical composition, the pharmaceutically acceptable excipient comprises a surfactant. In some cases of the pharmaceutical composition, the surfactant comprises Tween, sodium lauryl sulfate (SLS), or dipalmitoylphosphatidylcholine (DPPC). In some cases of the pharmaceutical composition, the pharmaceutically acceptable excipient comprises a lipid. In some cases of the pharmaceutical composition, the lipid comprises a polymeric lipid, a sulfonated poly saccharide, or a fatty acid. In some cases of the pharmaceutical composition, the lipid comprises a polymeric lipid, a sulfonated poly saccharide, or a fatty acid.

In some cases of the pharmaceutical composition, the solubility of the imatinib or derivative thereof in the aqueous solution is negatively correlated with the pH of the aqueous solution. In some cases of the pharmaceutical composition, the solubility of the imatinib or derivative thereof in the aqueous solution is positively correlated with concentration of the cyclodextrin in the aqueous solution.

In some aspects, disclosed herein is an aerosol composition, comprising nebulized droplets of the pharmaceutical composition disclosed herein, or nebulized droplets of the composition disclosed herein. In some cases, the nebulized droplets have an average mass median aerodynamic diameter of from 1 μm to 5 μm, from 1 μm to 4 μm, from 1 μm to 3 μm, from 1 μm to 2 μm, from 2 μm to 5 μm, from 2 μm to 4 μm, from 2 μm to 3 μm, or from 3 μm to 4 μm.

In some aspects, disclosed herein is a unit dose of the pharmaceutical composition disclosed herein, or the composition disclosed herein, or the aerosol composition disclosed herein, comprising from about 10 mg to about 500 mg of imatinib or a derivative thereof. In some cases, the unit dose comprises from 20 mg to 180 mg, from 20 mg to 150 mg, from 20 mg to 120 mg, from 20 mg to 100 mg, from 20 mg to 80 mg, from 20 mg to 60 mg, from 20 mg to 40 mg, from 40 mg to 120 mg, from 60 mg to 100 mg, or from 60 mg to 80 mg of the imatinib or derivative thereof.

In some aspects, disclosed herein is a method of treating a subject having a pulmonary disease, comprising administering to the subject in need thereof via inhalation the pharmaceutical composition disclosed herein.

In some cases, the method comprises administering to the subject from about 10 mg to about 500 mg of the imatinib or derivative thereof via inhalation. In some cases, the method comprises administering to the subject from 20 mg to 180 mg, from 20 mg to 150 mg, from 20 mg to 120 mg, from 20 mg to 100 mg, from 20 mg to 80 mg, from 20 mg to 60 mg, from 20 mg to 40 mg, from 40 mg to 120 mg, from 60 mg to 100 mg, or from 60 mg to 80 mg of the imatinib or derivative thereof.

In some cases, the pulmonary disease comprises lung fibrosis, lung cancer, or pulmonary hypertension. In some cases, the pulmonary disease comprises pulmonary arterial hypertension. In some cases, the method comprises administering to the subject the pharmaceutical composition at least once per day. In some cases, the method comprises administering to the subject the pharmaceutical composition 2, 3, 4, or 5 times per day. In some cases, the method comprises administering to the subject the pharmaceutical composition for a period of at least 5, 10, 20, 30, 60, 100, or 300 days, at least 1, 2, 3, 4, or 5 years.

In some cases of the method, the administering is performed using a nebulizer. In some cases of the method, the nebulizer is a jet nebulizer, a vibrating mesh nebulizer, or an ultrasonic nebulizer. In some cases of the method, administration of a single unit dose of the pharmaceutical composition takes place within 30 minutes. In some cases of the method, the administration of a single unit dosage of the pharmaceutical composition takes place within 15 minutes, 10 minutes, or 5 minutes. In some cases of the method, the administration of the pharmaceutical composition does not induce cough reflex of the subject. In some cases of the method, the pharmaceutical composition is not or minimally irritative to mouth or throat of the subject.

In some aspects, disclosed herein is a kit, comprising: the pharmaceutical composition disclosed herein or the unit dose disclosed herein, and instructions for use of the pharmaceutical composition for treatment of a pulmonary disease.

In some aspects, disclosed herein is a kit, comprising: (a) the pharmaceutical composition disclosed herein; (b) a receptacle containing the pharmaceutical composition; and (c) instructions for administering the pharmaceutical composition to a subject in need thereof via a nebulizer.

In some aspects, disclosed herein is a system comprising: the pharmaceutical composition disclosed herein and a nebulizer. In some cases of the system, the nebulizer is a jet nebulizer, a vibrating mesh nebulizer, or an ultrasonic nebulizer.

In some aspects, disclosed herein is a method of manufacturing a pharmaceutical composition that comprises imatinib or a derivative thereof, comprising: providing an aqueous solution comprising a solubility enhancer; dissolving the imatinib or derivative thereof, or a pharmaceutically acceptable salt thereof in the aqueous solution comprising the solubility enhancer, thereby producing an aqueous solution containing imatinib or derivative thereof; and adjusting volume, pH, osmolality, or viscosity of the aqueous solution containing imatinib or derivative thereof, thereby producing the pharmaceutical composition that comprises imatinib or derivative thereof.

In some cases of the method, the imatinib or derivative thereof, or pharmaceutically acceptable salt thereof comprises imatinib free base. In some cases of the method, the imatinib or derivative thereof, or pharmaceutically acceptable salt thereof is imatinib free base. In some cases of the method, the imatinib or derivative thereof, or pharmaceutically acceptable salt thereof comprises salt of imatinib selected from the group consisting of: acetate salt, formate salt, citrate salt, phosphate salt, maleate salt, fumarate salt, tartrate salt, malonate salt, lactic salt, and succinate salt. In some cases of the method, the imatinib or derivative thereof, or pharmaceutically acceptable salt thereof comprises less than 0.2%, less than 0.1%, less than 0.05%, less than 0.02%, less than 0.01%, or less than 0.001% imatinib mesylate. In some cases of the method, the pharmaceutical composition comprises less than 1 mg/mL, less than 0.5 mg/mL, less than 0.1 mg/mL, less than 0.005 mg/mL, less than 0.001 mg/mL, or less than 0.0001 mg/mL imatinib mesylate. In some cases of the method, the imatinib or derivative thereof, or pharmaceutically acceptable salt thereof does not comprise imatinib mesylate. In some cases of the method, the pharmaceutical composition does not comprise imatinib mesylate. In some cases of the method, the solubility enhancer is selected from the group consisting of: cyclodextrins, lipids, co-solvents, and organic acids.

In some cases of the method, the solubility enhancer comprises a cyclodextrin. In some cases of the method, the cyclodextrin is selected from the group consisting of: α-cyclodextrin, 3-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, 6A-amino-6A-deoxy-N-(3-hydroxypropyl)-β-cyclodextrin. In some cases of the method, the cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfobutylether-γ-cyclodextrin, or sulfobutylether-γ-cyclodextrin. In some cases of the method, the cyclodextrin comprises an anionic cyclodextrin. In some cases of the method, the cyclodextrin comprises hydroxypropyl-β-cyclodextrin. In some cases of the method, the cyclodextrin comprises hydroxypropyl-β-cyclodextrin. In some cases of the method, the aqueous solution comprises a salt of the cyclodextrin. In some cases of the method, salt of said cyclodextrin is a salt selected from the group consisting of: sodium salt, calcium salt, magnesium salt, iron salt, chromium salt, copper salt, zinc salt, lysine salt, arginine salt, and histidine salt. In some cases of the method, the aqueous solution comprises sulfobutylether-β-cyclodextrin sodium.

In some cases of the method, the pharmaceutical composition comprises the cyclodextrin at a concentration of from about 1% (w/v) to about 80% (w/v), from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v). In some cases of the method, the pharmaceutical composition comprises the cyclodextrin at a concentration of from 5% (w/v) to 40% (w/v). In some cases of the method, the pharmaceutical composition comprises the cyclodextrin at a concentration of from 10% (w/v) to 20% (w/v). In some cases of the method, the pharmaceutical composition comprises the cyclodextrin at a concentration of from 25% (w/v) to 40% (w/v). In some cases of the method, the pharmaceutical composition comprises the cyclodextrin at a concentration of about 10% (w/v), about 12% (w/v), about 14% (w/v), about 15% (w/v), about 16% (w/v), about 18% (w/v), or about 20% (w/v). In some cases of the method, the pharmaceutical composition comprises the cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

In some cases of the method, the solubility enhancer comprises a lipid or a fatty acid. In some cases of the method, the lipid or fatty acid is selected from the group consisting of: polyethoxylated castor oil, phospholipids, glycolipids, ganglioside GM1, sphingomyelin, phosphatidic acid, cardiolipin; lipids bearing polymer chains such as polyethylene glycol (PEG), chitin, hyaluronic acid, and polyvinylpyrrolidone; lipids bearing sulfonated monosaccharides, lipid-bearing sulfonated disaccharides, lipid bearing sulfonated polysaccharides; fatty acids such as palmitic acid, stearic acid, and oleic acid; cholesterol, cholesterol esters, and cholesterol hemisuccinate. In some cases of the method, the solubility enhancer comprises a co-solvent. In some cases of the method, the co-solvent comprises glycerol or ethanol.

In some cases of the method, the solubility enhancer comprises an organic acid. In some cases of the method, the organic acid is selected from the group consisting of: acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric Acid, tannic acid, L(+)-tartaric acid, taurocholic acid, and thiodipropionic acid.

In some cases of the method, the pharmaceutical composition comprises from 20 to 500 mg/mL of the imatinib or derivative thereof. In some cases of the method, the pharmaceutical composition comprises from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of the imatinib or derivative thereof. In some cases of the method, the pharmaceutical composition comprises about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of the imatinib or derivative thereof. In some cases of the method, the pharmaceutical composition comprises about 80 mg/mL of the imatinib or derivative thereof.

In some cases of the method, the aqueous solution comprising the solubility enhancer further comprises a pH buffer. In some cases of the method, the pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer. In some cases of the method, the pH buffer comprises a phosphate buffer. In some cases of the method, the pharmaceutical composition has a pH of 3 to 8. In some cases of the method, the pharmaceutical composition has a pH of from 3 to 6, from 4 to 6, from 4.5 to 5.5, from 5 to 6, from 4 to 7, from 5 to 7, or from 6 to 7. In some cases of the method, the pharmaceutical composition has a pH of about 4.7, about 4.8, about 4.9, about 5.0, about 5.1, about 5.2, about 5.4, about 5.5, or about 5.6. In some cases of the method, the pharmaceutical composition has a pH of from 7 to 8. In some cases of the method, the pharmaceutical composition has a pH of about 7.0, about 7.2, about 7.4, about 7.6, about 7.8, or about 8.0.

In some cases of the method, the pharmaceutical composition has a viscosity of at most 10 centipoise. In some cases of the method, the pharmaceutical composition further comprises a pharmaceutically acceptable excipient. In some cases of the method, the pharmaceutically acceptable excipient comprises a surfactant. In some cases of the method, the surfactant comprises Tween, sodium lauryl sulfate (SLS), or dipalmitoylphosphatidylcholine (DPPC). In some cases of the method, the pharmaceutically acceptable excipient comprises a lipid. In some cases of the method, the lipid comprises a polymeric lipid, a sulfonated poly saccharide, or a fatty acid. In some cases of the method, the lipid comprises a polymeric lipid, a sulfonated poly saccharide, or a fatty acid. In some cases of the method, the solubility of the imatinib or derivative thereof in the aqueous solution is negatively correlated with the pH of the aqueous solution. In some cases of the method, the solubility of the imatinib or derivative thereof in the aqueous solution is positively correlated with concentration of the cyclodextrin in the aqueous solution.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure may be obtained by reference to the following detailed description that sets forth illustrative embodiments, in which the principles of the disclosure are utilized, and the accompanying drawings of which:

FIG. 1 shows a graph demonstrating the maximum concentration of imatinib free base (mg/mL) as a function of pH.

FIG. 2 shows a graph demonstrating the maximum concentration of imatinib free base (mg/mL) as a function of percent hydroxypropyl β cyclodextrin (HPβCD or “HPBCD” in the figure) (w/v) at a pH of 5 and 7.5.

FIG. 3A shows a graph demonstrating the maximum concentration of imatinib free base (mg/mL) as a function of percent hydroxypropyl β cyclodextrin (HPβCD or “HPBCD” in the figure) (w/v) at a pH of 5 and 7.5 and percent sulfobutylether β cyclodextrin (SBEβCD or “SBEBCD” in the figure) (w/v) at a pH of 5.

FIG. 3B shows pictures of exemplary suspension and solutions of about 30 mg/mL imatinib free base in an aqueous solution of 30% SBEβCD and 50 mM phosphate buffer at different pH levels.

FIG. 4A is a plot summarizing lung tissue concentration of imatinib post IT (freebase suspension) or IV (mesylate solution) administration over time.

FIG. 4B is a plot summarizing plasma concentration of imatinib post IT (freebase suspension) or IV (mesylate solution) administration over time.

FIG. 4C is a plot summarizing lung tissue concentration of imatinib vs. plasma concentration of imatinib over time post IT administration of the imatinib free base suspension.

FIG. 4D is a plot summarizing lung tissue concentration of imatinib vs. plasma concentration of imatinib over time post IV administration of the imatinib mesylate solution.

FIG. 4E is a plot summarizing lung tissue concentration of imatinib vs. plasma concentration of imatinib over time post IT administration of the imatinib free base suspension plotted on a log scale.

FIG. 4F is a plot on a log scale summarizing lung tissue concentration of imatinib vs. plasma concentration of imatinib over time post IV administration of the imatinib mesylate solution.

### DETAILED DESCRIPTION

In some aspects, the present disclosure provides compositions (e.g., pharmaceutical compositions), methods (e.g., methods of treatment, methods of making the compositions), kits, and systems that relate to an aqueous solution or suspension of imatinib. The aqueous solution or suspension of imatinib disclosed herein can be used as an inhalable formulation, e.g., via aerosolization by a nebulizer, for use in human patients. In some embodiments, the pharmaceutical compositions and methods of treatment provided herein are advantageous in offering fast, efficient, and safe therapeutic solution to treating pulmonary conditions. In some embodiments, the present disclosure relates to inhalational administration of a pharmaceutical composition in an aqueous solution or suspension form that comprises imatinib and a solubility enhancer.

In some embodiments, the pharmaceutical composition or formulation provided herein enables delivery of more pharmaceutically active ingredient, e.g., imatinib, to the subject, in a single dose, or in multiples doses over a period of time. In some embodiments, the subject pharmaceutical composition or formulation has at least one solubility enhancer. In some embodiments, the solubility enhancers comprises cyclodextrin, pH buffer, lipids, fatty acids, co-solvents, or organic solvents. In some cases, the pharmaceutical compositions described herein has a variable concentration of solubility enhancers to increase solubility of imatinib or a derivative thereof, or a pharmaceutically acceptable salt thereof. As used herein, the term “solubility” with respect to a designated solute in a solution can refer to the maximum amount of the solute that can be dissolved in a unit amount of a designated solvent in the solution. The term “solubility” when used with reference to imatinib or a derivative thereof (e.g., imatinib free base) that is dissolved in an aqueous solution can refer to the maximum amount of imatinib or derivative thereof that can be dissolved in a unit amount of water present in the aqueous solution. In some embodiments, the amount of imatinib to solubility enhancers in the aqueous solution or suspension provided herein shortens the inhalation duration as a given dose can be delivered at a higher speed, for instance, as compared to a comparable formulation that does not have the solubility enhancer, and thus has a relatively much lower concentration of imatinib. Shorter inhalation duration can improve subject compliance, which can further increase the delivery efficiency of the drug.

In some embodiments, the pharmaceutical composition or formulation provided herein reduces adverse cough of the subject while inhaling, has improved organoleptic properties, and improves overall patient experience of inhalation. In some embodiments, the improved overall inhalation experience results in better compliance with the full inhalation program. In some embodiments, more effective drug delivery is achieved when the subject has better inhalation compliance, and thus more drug is delivered. Some aqueous solutions of imatinib mesylate or other salt of imatinib can have poor organoleptic properties, for instance, they can be severely irritative to respiratory tract, can induce significant adverse sensation in mouth and throat when inhaled by a human subject, and/or can induce cough or even strong coughs so that continuous deep lung inhalation may become impossible or impractical. In some cases, solutions of imatinib mesylate or certain other salts of imatinib are not inhalable, e.g., because they are not organoleptically tolerable to human subjects to enable continuous deep lung inhalation of the nebulized aerosol. In contrast, formulations according to some embodiments of the present disclosure can have improved organoleptic properties and suitable for deep lung inhalation of their nebulized aerosols. In some cases, formulations provided herein, for instance, aqueous solutions made of imatinib freebase, are not irritative or minimally irritative to mouth and throat when being inhaled in the form of a nebulized aerosol. For instance, subject may not experience any adverse or severely adverse sensational irritation when inhaling nebulized aerosol of some formulations provided herein. Some formulations provided herein may not induce cough reflex or strong coughs of the subject inhaling the formulations. Subject inhaling some formulations provided herein may report some formulations provided herein as tolerable and can continuously conduct deep lung inhalation of them for a desirable period of time.

Without being bound by a certain theory, mesylate salt form of imatinib may contribute to the adverse organoleptic properties of the nebulized aerosol of its aqueous solution. In some embodiments, the formulations provided herein circumvent the problem associated with the adverse organoleptic properties by preparing the formulation using imatinib freebase or other salt forms of imatinib. The absence of mesylate form of imatinib can contribute to the improved organoleptic properties of certain formulations provided herein.

In one aspect of the present disclosure, provided herein is a unit dose of a pharmaceutical composition provided herein. In some embodiments, the unit dose comprises about 20 mg to about 500 mg of imatinib free base. In another aspect, provided herein are kits comprising the pharmaceutical composition or the unit dose provided herein and instructions for use of the pharmaceutical composition for treatment of a pulmonary disease.

### Composition

In some aspects, provided herein are compositions (e.g., pharmaceutical compositions or formulations) that comprise an aqueous solution or suspension of imatinib or a derivative thereof. In some cases, the composition comprises an aqueous solution or suspension that comprises: imatinib or a derivative thereof, a solubility enhancer, and a pH buffer. In some embodiments, the aqueous solution or suspension has a concentration of imatinib of from 20 to 500 mg/mL. In some cases, the aqueous solution or suspension has a viscosity of at most 10 centipoise. In some cases, the aqueous solution or suspension has a pH of 3 to 8. In some cases, the aqueous solution or suspension has a concentration of imatinib or derivative thereof of from 20 to 500 mg/m, has a viscosity of at most 10 centipoise and has a pH of 3 to 8.

In some cases, provided herein is a composition that comprises an aqueous solution or suspension that comprises imatinib or a derivative thereof, and cyclodextrin. In some cases, the aqueous solution or suspension has the cyclodextrin at a concentration of from about 1% (w/v) to about 80% (w/v). In some cases, the cyclodextrin is anionic cyclodextrin.

In some cases, the compositions provided herein have from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL imatinib or derivative thereof.

In some cases, the compositions provided herein have a viscosity of at most 10 centipoise, such as at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise. For instance, the composition provided herein has a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.

In some cases, the compositions provided herein have a pH of 3 to 7, such as from 3 to 6, from 4 to 6, from 4.5 to 5.5, from 5 to 6, from 4 to 7, from 5 to 7, or from 6 to 7. For instance, the composition provided herein has a pH of about 4.5, about 4.7, about 4.8, about 4.9, about 5.0, about 5.1, about 5.2, about 5.4, about 5.5, or about 5.6. In some cases, the composition comprises an aqueous solution of imatinib or derivative thereof, and has a pH of 3 to 7.

In some cases, the compositions provided herein have a pH of 7 to 8, such as about about 7.0, about 7.2, about 7.4, about 7.6, about 7.8, or about 8.0. In some cases, the composition comprises an aqueous suspension of imatinib or derivative thereof, and has a pH of 3 to 8.

In some cases, the compositions provided herein do not have imatinib mesylate. In some cases, the compositions provided herein have substantially low amount of imatinib mesylate, for instance, less than 1 mg/mL, less than 0.5 mg/mL, less than 0.4 mg/mL, less than 0.3 mg/mL, less than 0.2 mg/mL, less than 0.1 mg/mL, less than 0.075 mg/mL, less than 0.05 mg/mL, less than 0.025 mg/mL, less than 0.01 mg/mL, less than 0.0075 mg/mL, less than 0.005 mg/mL, less than 0.0025 mg/mL, less than 0.001 mg/mL, less than 0.00075 mg/mL, less than 0.0005 mg/mL, less than 0.00025 mg/mL, or less than 0.0001 mg/mL imatinib mesylate, or less. In some cases, the composition has less than less than 0.2%, less than 0.15%, less than 0.1%, less than 0.075%, less than 0.05%, less than 0.025%, less than 0.02%, less than 0.015%, less than 0.01%, less than 0.0075%, less than 0.005%, less than 0.0025%, less than 0.002%, less than 0.0015%, or less than 0.001% imatinib mesylate, or even less in total amount of imatinib contained in the composition.

In some cases, the composition provided herein comprises an aqueous solution of imatinib or a derivative thereof. The term “solution,” as used herein, can refer to a homogenous mixture of one or more solutes dissolved in a solvent. The term “solvent” can refer to the substance in which a solute dissolves to produce the homogeneous mixture, and the term “solute” can refer to the substance that dissolves in a solvent to produce the homogeneous mixture. As used herein, the term “aqueous solution” can refer to a solution in which one of the one or more solvents is water.

In some cases, the composition provided herein is an aqueous suspension of imatinib or a derivative thereof. The term “suspension,” as used herein, can refer to a heterogenous mixture in which at least some of the solute particles do not dissolve, but get suspended throughout the bulk of the solvent. The suspension disclosed herein can have some of the solute (e.g., imatinib or a derivative thereof) dissolved in the solvent (e.g., water) while the remainder suspended in the water, left floating around freely therein. The term “aqueous suspension,” as used herein, can refer to a suspension in which one of the one or more solvents is water. The suspension provided herein may be used for therapeutic treatment and may be prepared (e.g., mixing the components and suspending the undissolved imatinib or derivative thereof in the water-based solution) immediately prior to the therapeutic use.

Imatinib

In some aspects, the composition disclosed herein comprises imatinib or a derivative thereof. In some aspects of the present disclosure, the pharmaceutical composition comprises a pharmaceutical agent for treating pulmonary diseases. In some embodiments, the pharmaceutical agent for treating pulmonary diseases is imatinib or a pharmaceutically acceptable salt thereof. In some embodiments, the pharmaceutically active ingredient in the composition (e.g., pharmaceutical composition or formulation) is imatinib free base. In some embodiments, the pharmaceutically active ingredient, e.g., the pharmaceutically acceptable salt of imatinib, comprises imatinib mesylate. In some embodiments, the pharmaceutical agent for treating pulmonary diseases is an imatinib derivative (e.g., Nilotinib, Sorafenib, Dasatinib) or a pharmaceutically acceptable salt thereof. Exemplary imatinib derivatives can include those that are described in Skobridis K et al. Chem Med Chem. 2010 January; 5(1):130-9, A. Mortlock et al. Comprehensive Medicinal Chemistry II, Volume 7, 2007, Pages 183-220, and Musumeci F et al. Expert Opin Ther Pat. 2015; 25(12):1411-21, all of which are incorporated herein in its entirety.

The chemical structure of imatinib is shown in Compound I.

### Compound I

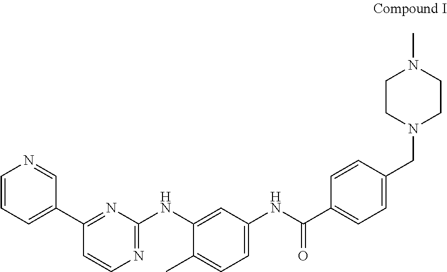 [SMILES: Cc1ccc(NC(=O)c2ccc(CN3CCN(C)CC3)cc2)cc1Nc1nccc(-c2cccnc2)n1]

Examples of pharmaceutically acceptable salts include those salts prepared by reaction of the composition described herein with a mineral acid, organic acid, or inorganic base, such salts including acetate, acrylate, adipate, alginate, aspartate, benzoate, benzenesulfonate, bisulfate, bisulfate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the composition (e.g., pharmaceutical composition or formulation) described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those pharmaceutical compositions described herein, which comprise a free acid group, react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

In some embodiments, the pharmaceutically acceptable salt of imatinib comprises acetate salt, formate salt, citrate salt, phosphate salt, maleate salt, fumarate salt, tartrate salt, malonate salt, lactic salt, and succinate salt.

Solubility Enhancer

In some aspects of the present disclosure, a solubility enhancer described herein provides the pharmaceutical agent for treating pulmonary diseases, e.g., imatinib or a derivative thereof, or a pharmaceutically acceptable salt thereof, increased solubility in an aqueous solution. In some aspects of the present disclosure, a solubility enhancer described herein is a cyclodextrin. In some aspects of the present disclosure, a solubility enhancer described herein is a lipid or a fatty acid. In some aspects of the present disclosure, a solubility enhancer described herein is a co-solvent. In some aspects of the present disclosure, a solubility enhancer described herein is an organic acid or generally recognized as safe (GRAS) excipient acid. In some aspects of the present disclosure, a solubility enhancer described herein is a surfactant, such as Tween, sodium lauryl sulfate (SLS), or dipalmitoylphosphatidylcholine (DPPC).

The lipid or fatty acid in a composition (e.g., pharmaceutical composition or formulation) provided herein can include, but not limited to, polyethoxylated castor oil, phospholipids, glycolipids, ganglioside GM1, sphingomyelin, phosphatidic acid, cardiolipin; lipids bearing polymer chains such as polyethylene glycol (PEG, PEG300, PEG400), propylene glycol (PG), chitin, hyaluronic acid, and polyvinylpyrrolidone; lipids bearing sulfonated monosaccharides, lipid-bearing sulfonated disaccharides, lipid bearing sulfonated polysaccharides; fatty acids such as palmitic acid, stearic acid, and oleic acid; cholesterol, cholesterol esters, and cholesterol hemisuccinate. In some aspects of the present disclosure, the lipid is polymeric. In some aspects of the present disclosure, the polymeric lipid is polyvinylpyrrolidone (PVP). In some aspects of the present disclosure, the polymeric lipid is polyethylene glycol (PEG). In some aspects of the present disclosure, the lipid is a sulfonated polysaccharide. In some aspects of the present disclosure, the lipid is a fatty acid. In some aspects of the present disclosure, the fatty acid is steric or oleic acid. In some aspects of the present disclosure, the fatty acid a phospholipid. In some embodiments, the phospholipid is lecirhin or 1,2-dipalmitoyl-sn-glycerol-3-phosphocholine (DPPC).

The co-solvent in a composition (e.g., pharmaceutical composition or formulation) provided herein can include, but not limited to, glycol or ethanol. The organic acid in a composition (e.g., pharmaceutical composition or formulation) provided herein can include, but not limited to acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric acid, tannic acid, L(+)-tartaric acid, taurocholic acid, and thiodipropionic acid. The GRAS excipient acid in a composition (e.g., pharmaceutical composition or formulation) provided herein can include, but not limited to, acetic acid, formic acid, citrate, tartrate, maleate, fumarate, tartrate, malonate, lactic, and succinate.

Cyclodextrin

In some aspects of the present disclosure, a cyclodextrin is used as a solubility enhancer of imatinib or a derivative thereof. In some aspects of the present disclosure, a cyclodextrin is used as a solubility enhancer of imatinib free base. In some aspects of the present disclosure, a cyclodextrin is used as a solubility enhancer of a salt of imatinib. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit can have secondary hydroxyl groups at the 2 and 3 positions and a primary hydroxyl group at the 6-position. The cyclodextrins can be pictured as hollow truncated cones with hydrophilic exterior surfaces and hydrophobic interior cavities. In aqueous solutions, these hydrophobic cavities can provide a haven for hydrophobic organic compounds that can fit all or part of their structure into these cavities. This process, known as inclusion complexation, can result in increased apparent aqueous solubility and stability for the complexed drug.

The cyclodextrin in a composition (e.g., a pharmaceutical composition) provided herein can include, but not limited to, α-cyclodextrin (αCD), β-cyclodextrin (βCD), γ-cyclodextrin (γCD), derivatized α-cyclodextrins, derivatized β-cyclodextrins, and derivatized γ-cyclodextrins. Non-limiting examples of cyclodextrin that can be used in the subject composition (e.g., pharmaceutical composition or formulation) include α-cyclodextrin, β-cyclodextrin, γ-cyclodextrin, hydroxypropyl-β-cyclodextrin (HPβCD), hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin (MβCD), succinyl-α-cyclodextrin (SαCD), succinyl-β-cyclodextrin (SβCD), succinyl-γ-cyclodextrin (SγCD), 6A-amino-6A-deoxy-N-(3-hydroxypropyl)-β-cyclodextrin, sulfobutylether-α-cyclodextrin (SBEαCD) sulfobutylether-β-cyclodextrin (SBEβCD), sulfobutylether-γ-cyclodextrin, sulfoalkylether-β-cyclodextrins, sulfoalkylether-γ-cyclodextrins, carboxymethyl-α-cyclodextrin (CMαCD), carboxymethyl-β-cyclodextrin (CMβCD), carboxymethyl-γ-cyclodextrin (CMγCD), 2-carboxyethyl-α-cyclodextrin (CEαCD), 2-carboxyethyl-β-cyclodextrin (CEβCD), 2-carboxyethyl-γ-cyclodextrin (CEαCD), phosphate-α-cyclodextrin (PαCD), phosphate-β-cyclodextrin (PβCD), and phosphate-γ-cyclodextrin (PγCD). In some embodiments, the composition (e.g., the pharmaceutical composition) comprises hydroxypropyl-β-cyclodextrin (HPβCD). In some embodiments, the composition (e.g., the pharmaceutical composition) comprises more than one species of cyclodextrins, such as, 2, 3, 4, 5, 6, 7, 8, 9, 10 or more different species of cyclodextrins. In some embodiments, the composition (e.g., the pharmaceutical composition) comprises HPβCD and one or more other cyclodextrins, such as, 1, 2, 3, 4, 5, 6, 7, 8, 9, 10 or more other different species of cyclodextrins. In some cases, the cyclodextrin is selected from the group consisting of: α-cyclodextrin, β-cyclodextrin, γ-cyclodextrin, derivatized α-cyclodextrins, derivatized 3-cyclodextrins, and derivatized γ-cyclodextrins. In some cases, the cyclodextrin is selected from the group consisting of: α-cyclodextrin, β-cyclodextrin, γ-cyclodextrin, hydroxypropyl-3-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, succinyl-β-cyclodextrin, 6A-amino-6A-deoxy-N-(3-hydroxypropyl)-β-cyclodextrin, sulfobutylether-β-cyclodextrin, sulfobutylether-γ-cyclodextrin, sulfoalkylether-β-cyclodextrins, and sulfoalkylether-γ-cyclodextrins.

In some cases, a salt of cyclodextrin is used to prepare the composition disclosed herein. For instance, a metal salt of cyclodextrin can be used, such as, but not limited to, sodium salt, calcium salt, magnesium salt, iron salt, chromium salt, copper salt, and zinc salt. Alternatively or additionally, an amino acid salt of cyclodextrin can be used, such as, but not limited to, lysine, arginine, and histidine salts. In some cases, the aqueous solution or suspension disclosed herein comprises a salt of a cyclodextrin, for instance, a salt of an anionic cyclodextrin, e.g., a salt of sulfobutylether-β-cyclodextrin. In some cases, the aqueous solution or suspension disclosed herein comprises sulfobutylether-β-cyclodextrin sodium.

In some embodiments, the concentration of the cyclodextrin contributes to the viscosity of the solution, which can reduce the nebulization efficiency (or rate) of the solution. For instance, in some cases, the higher the concentration of the cyclodextrin is, the higher viscosity of the solution is. In some cases, the concentration of the cyclodextrin in the composition is controlled so that the viscosity of the solution is not higher than a reference value, such as about 0.1 centipoise (cP), 0.2 cP, 0.3 cP, 0.4 cP, 0.5 cP, 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.2 cP, 3.4 cP, 3.5 cP, 3.6 cP, 3.8 cP, 4.0 cP, 4.2 cP, 4.4 cP, 4.6 cP, 4.8 cP, 5.0 cP, 5.2 cP, 5.4 cP, 5.6 cP, 5.8 cP, 6.0 cP, 6.2 cP, 6.4 cP, 6.6 cP, 6.8 cP, 7.0 cP, 7.2 cP, 7.4 cP, 7.6 cP, 7.8 cP, 8.0 cP, 8.2 cP, 8.4 cP, 8.6 cP, 8.8 cP, 9.0 cP, 9.2 cP, 9.4 cP, 9.6 cP, 9.8 cP, or 10.0 cP. In some cases, the concentration of the cyclodextrin in the composition (e.g., pharmaceutical composition or formulation) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution.

Without wishing to be bound to a certain theory, imatinib or a derivative thereof (e.g., imatinib free base) in acidic conditions can be protonated, leading to an increase in the solubility of imatinib free base. Without wishing to be bound to a certain theory, cyclodextrin can increase the solubility of imatinib or a derivative thereof (e.g., imatinib free base) by stabilizing the positively charged piperazine ring in acidic conditions. Without wishing to be bound to a certain theory, cyclodextrins that are anionic can increase the stabilization of the positively charged imatinib or a derivative thereof (e.g., positively charged imatinib free base) and increasing the solubility. In some embodiments, cyclodextrins that can be anionic can be used in the compositions and methods disclosed herein, for example, succinyl-α-cyclodextrin (SαCD), succinyl-β-cyclodextrin (SβCD), succinyl-γ-cyclodextrin (SγCD), sulfobutylether-α-cyclodextrin (SBEαCD) sulfobutylether-β-cyclodextrin (SBEβCD), sulfobutylether-γ-cyclodextrin (SBEγCD), carboxymethyl-α-cyclodextrin (CMαCD), carboxymethyl-β-cyclodextrin (CMβCD), carboxymethyl-γ-cyclodextrin (CMγCD), 2-carboxyethyl-α-cyclodextrin (CEαCD), 2-carboxyethyl-β-cyclodextrin (CEβCD), 2-carboxyethyl-γ-cyclodextrin (CEαCD), phosphate-α-cyclodextrin (PαCD), phosphate-β-cyclodextrin (PβCD), and γ-cyclodextrin (PγCD).

In some aspects of the present disclosure, cyclodextrin, e.g., SBEβCD, is used as a solubility enhancer of imatinib or a derivative thereof. In some aspects of the present disclosure, cyclodextrin, e.g., SBEβCD, is used as a solubility enhancer of imatinib free base. In some aspects of the present disclosure, cyclodextrin, e.g., SBEβCD, is used as a solubility enhancer of a salt of imatinib. In some cases, the concentration of the cyclodextrin, e.g., SBEβCD, in the composition (e.g., the pharmaceutical composition) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution at a pH from about 3 to about 8. In some cases, the concentration of the cyclodextrin, e.g., SBEβCD, in the composition (e.g., the pharmaceutical composition) is at most about 20% (w/v) of the solution at a pH from about 3 to about 8. In some embodiments, the concentration of cyclodextrin, e.g., SBEβCD, in the composition is at about 2% to about 80% the solution at a pH of about 5. In some embodiments, the concentration of cyclodextrin, e.g., SBEβCD, in the composition is at about 2% to about 60% the solution at a pH of about 5. In some embodiments, the concentration of cyclodextrin, e.g., SBEβCD, in the composition is at about 2% to about 50% the solution at a pH of about 5. In some embodiments, the concentration of cyclodextrin, e.g., SBEβCD, in the composition is at about 5% to about 45% the solution at a pH of about 5. In some embodiments, the concentration of cyclodextrin, e.g., SBEβCD, in the composition is at about 10% to about 20% the solution at a pH of about 4. In some embodiments, the concentration of cyclodextrin, e.g., SBEβCD, in the composition is at about 2% to about 80% the solution at a pH of about 4. In some embodiments, the concentration of cyclodextrin, e.g., SBEβCD, in the composition is at about 2% to about 60% the solution at a pH of about 4. In some embodiments, the concentration of cyclodextrin, e.g., SBEβCD, in the composition is at about 2% to about 50% the solution at a pH of about 4. In some embodiments, the concentration of cyclodextrin, e.g., SBEβCD, in the composition is at about 5% to about 45% the solution at a pH of about 4. In some embodiments, the concentration of cyclodextrin, e.g., SBEβCD, in the composition is at about 10% to about 20% the solution at a pH of about 4. In some embodiments, the concentration of cyclodextrin, e.g., SBEβCD, in the composition (e.g., pharmaceutical composition or formulation) is at about 2% to about 80% the solution at a pH of about 3. In some embodiments, the concentration of cyclodextrin, e.g., SBEβCD, in the composition (e.g., pharmaceutical composition or formulation) is at about 2% to about 60% the solution at a pH of about 3. In some embodiments, the concentration of cyclodextrin, e.g., SBEβCD, in the composition is at about 2% to about 50% the solution at a pH of about 3. In some embodiments, the concentration of cyclodextrin, e.g., SBEβCD, in the composition is at about 5% to about 45% the solution at a pH of about 3. In some embodiments, the concentration of cyclodextrin, e.g., SBEβCD, in the composition is at about 10% to about 20% the solution at a pH of about 3. In some embodiments, the concentration of cyclodextrin, e.g., SBEβCD, in the composition is at about 2% to about 80% the solution at a pH of about 6. In some embodiments, the concentration of cyclodextrin, e.g., SBEβCD, in the composition is at about 2% to about 60% the solution at a pH of about 6. In some embodiments, the concentration of cyclodextrin, e.g., SBEβCD, in the composition is at about 2% to about 50% the solution at a pH of about 6. In some embodiments, the concentration of cyclodextrin, e.g., SBEβCD, in the composition is at about 5% to about 45% the solution at a pH of about 6. In some embodiments, the concentration of cyclodextrin, e.g., SBEβCD, in the composition is at about 10% to about 20% the solution at a pH of about 6.

In some cases, the concentration of the cyclodextrin, e.g., SBEβCD, in the composition (e.g., the pharmaceutical composition) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution or suspension at a pH from about 3 to about 8 and the concentration of imatinib dissolved is about 0.0001 mg/mL to about 500 mg/mL. In some cases, the concentration of the cyclodextrin, e.g., SBEβCD, in the composition is at most about 2% to about 80% (w/v) of the solution at a pH at about 5 and the concentration of imatinib dissolved is about 1 mg/mL to about 200 mg/mL.

In some cases, the formulation disclosed herein, e.g. a formulation containing imatinib free base and cyclodextrin e.g., SBEβCD, at an acidic pH range can lead to low systemic absorption of imatinib and a high lung residence time for imatinib. In some cases, the formulation disclosed herein, e.g. a formulation containing imatinib free base and cyclodextrin e.g., SBEβCD, at an acidic pH range can lead to highly preferential retention of the pharmaceutically active ingredient, e.g., imatinib or derivative thereof, in the lungs as compared to in plasma. Without wishing to be bound to a certain theory, minimizing systemic exposure can lead to decreasing systemic side effects. In some cases, an acidic solution of imatinib or derivative thereof, e.g. containing imatinib free base and cyclodextrin (e.g., SBEβCD), when administered to the lungs results in precipitation of imatinib or its salt upon coming in contact with the lung lining fluid.

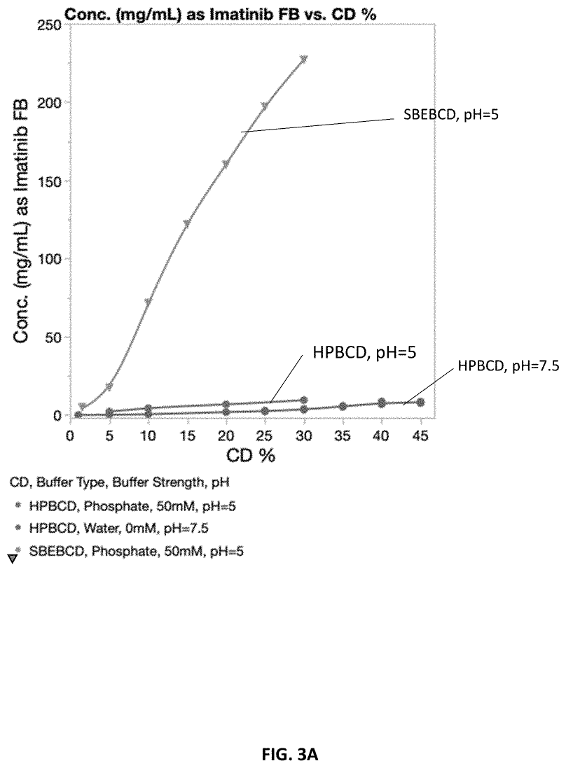

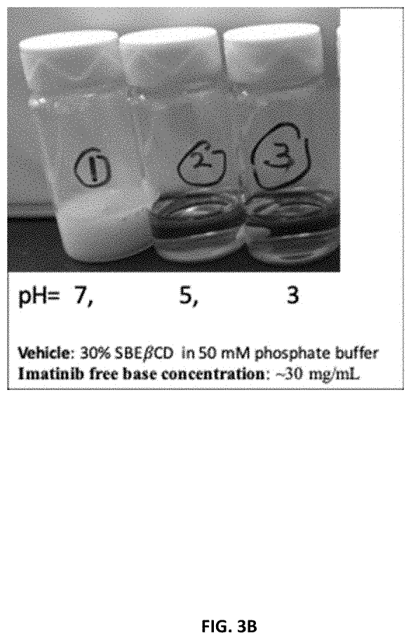

Solubility of imatinib or derivative thereof (e.g., imatinib free base) in an acidic solution according to some embodiments of the present disclosure can be negatively correlated with the pH of the solution, for instance, the higher the pH of the solution is, the lower the possibility that imatinib free base is dissolved in the solution is (e.g., as shown in Examples 4 and 5 and FIGS. 2, 3A, and 3B). Additionally or alternatively, solubility of imatinib or derivative thereof (e.g., imatinib free base) in an acidic solution containing cyclodextrin (e.g., SBE®CD) according to some embodiments of the present disclosure can be positively correlated with the concentration of cyclodextrin in the solution, for instance, the higher the concentration of SBE®CD is, the higher the possibility that imatinib freebase is dissolved in the solution is (e.g., as shown in Examples 5-7). Without wishing to be bound to a certain theory, in some cases, the lung lining fluid acts as a buffer that increases the pH to precipitate imatinib or a derivative thereof delivered in the formulation. Without wishing to be bound to a certain theory, in some cases, the lung lining fluid acts as a diluent to precipitate imatinib or a derivative thereof delivered in the formulation. Without wishing to be bound to a certain theory, in some cases, the lung lining fluid acts as a buffer increasing the pH and as a diluent to precipitate imatinib or a derivative thereof delivered in the formulation. Without wishing to be bound to a certain theory, solid precipitated imatinib can lead to extended release of imatinib to the lung tissue over time, lower systemic absorption, and longer lung residence time.

In some cases, within 10 min, 20 min, 30 min, 1 hour, 2 hours, 3 hours, 5 hours, or 10 hours after inhalatory administration of the exemplary composition disclosed herein, concentration of imatinib or derivative thereof in systemic circulation, e.g., concentration of imatinib measured in blood samples collected from large blood vessels or heart, is lower than concentration of imatinib in the lung tissue, e.g., concentration of imatinib measured in samples collected from the lung. For instance, within 10 min, 20 min, 30 min, 1 hour, 2 hours, 3 hours, 5 hours, or 10 hours after inhalatory administration, the concentration ratio of imatinib measured in blood sample compared to samples collected from the lungs is less than 1, such as about 0.0001 to about 0.9, about 0.001 to about 0.9, about 0.01 to about 0.9, or about 0.1 to about 0.9. In some embodiments, the concentration ratio is about 0.0001, about 0.0005, about 0.001, about 0.005, about 0.008, about 0.01, about 0.015, about 0.02, about 0.03, about 0.04, about 0.05, bout 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, or about 0.9.

In some cases, after inhalatory administration of the exemplary imatinib composition disclosed herein, concentration of imatinib in the lung tissues, e.g., concentration of imatinib measured in samples collected from the lung, decreases at a lower speed as compared to the concentration of imatinib in systemic circulation, e.g., concentration of imatinib measured in blood samples collected from large blood vessels or heart.

pH Buffers

In some aspects of the present disclosure, the compositions provided herein further comprises a pH buffer. In some cases, the pH buffer is an organic acid salt, including but not limited to, of citric acid, lactic acid, ascorbic acid, gluconic acid, carbonic acid, tartaric acid, succinic acid, acetic acid, or phthalic acid, Tris, tromethamine hydrochloride, or a phosphate buffer. In some embodiments, the composition comprise more than one pH buffer.

In some embodiments, the pH buffers are present in the compositions (e.g., pharmaceutical compositions) described herein to provide the aqueous solution a pH of about 3 to about 7. In some embodiments, the pH buffers are present in the compositions (e.g., pharmaceutical compositions) described herein to provide the aqueous suspension a pH of about 3 to about 8. In some cases, the pH of the composition (e.g., pharmaceutical composition or formulation) is about 3, about 4, about 5, about 6, about 7, or about 8 In some embodiments, the pH buffers are present in the compositions described herein to provide the aqueous solution a pH of the aqueous solution is from 3 to 6, from 4 to 6, from 4.5 to 5.5, from 5 to 6, from 4 to 7, from 5 to 7, or from 6 to 7.

In some embodiments, the pH buffer is present in the aqueous composition at about 0.001 mg/mL to about 100 mg/mL, for example between about 0.1 mg/mL to about 100 mg/mL, about 0.5 mg/mL to about 50 mg/mL, about 0.5 mg/mL to about 20 mg/ml, about 0.5 mg/mL to about 10 mg/mL, about 0.5 mg/mL to 5 mg/mL, or about 1 mg/mL to about 5 mg/mL. In some embodiments, the pH buffer is present in the aqueous composition at about 0.1 mM to about 500 mM, for example from about 1 mM to 500 mM, 1 mM to 200 mM, 1 mM to 100 mM, 1 mM to 80 mM, 1 mM to 50 mM, 1 mM to 25 mM, 1 mM to 10 mM, 1 mM to 5 mM, 5 mM to 200 mM, 5 mM to 100 mM, 5 mM to 80 mM, 5 mM to 50 mM, 5 mM to 25 mM, 5 mM to 10 mM, 20 mM to 200 mM, 20 mM to 100 mM, 20 mM to 80 mM, or 20 mM to 50 mM.

Pharmaceutical Acceptable Excipients

In one aspect, provided herein are formulations for treatment of a pulmonary disease. The formulations can include the compositions provided herein and a pharmaceutically acceptable carrier, excipient, diluent, or any other suitable component for the intended administration routes, such as oral or nasal inhalation. Examples of pharmaceutically acceptable excipients include, but are not limited to, lipids, metal ions, surfactants, amino acids, carbohydrates, buffers, salts, polymers, sweeteners, and the like, and combinations thereof.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose, and the like; disaccharides such as lactose, maltose, sucrose, trehalose, and the like; trisaccharides such as raffinose and the like; and other carbohydrates such as starches (hydroxyethylstarch), and maltodextrins.

Non-limiting examples of lipids include phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate.

In some cases, the phospholipid comprises a saturated phospholipid, such as one or more phosphatidylcholines. Exemplary acyl chain lengths are 16:0 and 18:0 (e.g., palmitoyl and stearoyl). The phospholipid content can be determined by the active agent activity, the mode of delivery, and other factors.

Phospholipids from both natural and synthetic sources can be used in varying amounts. When phospholipids are present, the amount is typically sufficient to coat the active agent(s) with at least a single molecular layer of phospholipid. In general, the phospholipid content ranges from about 5 wt % to about 99.9 wt %, such as about 20 wt % to about 80 wt %.

Generally, compatible phospholipids can comprise those that have a gel to liquid crystal phase transition greater than about 40° C., such as greater than about 60° C., or greater than about 80° C. The incorporated phospholipids can be relatively long chain (e.g., C₁₆-C₂₂) saturated lipids. Exemplary phospholipids useful in the present disclosure include, but are not limited to, phosphoglycerides such as dipalmitoylphosphatidylcholine, distearoylphosphatidylcholine, diarachidoylphosphatidylcholine, dibehenoylphosphatidylcholine, diphosphatidyl glycerols, short-chain phosphatidylcholines, hydrogenated phosphatidylcholine, E-100-3 (available from Lipoid KG, Ludwigshafen, Germany), long-chain saturated phosphatidylethanolamines, long-chain saturated phosphatidylserines, long-chain saturated phosphatidylglycerols, long-chain saturated phosphatidylinositols, phosphatidic acid, phosphatidylinositol, and sphingomyelin.

Examples of metal ions include, but are not limited to, divalent cations, including calcium, magnesium, zinc, iron, and the like. For instance, when phospholipids are used, the pharmaceutical composition can also comprise a polyvalent cation, as disclosed in U.S. Pat. Nos. 8,709,484 and 7,871,598, which are incorporated herein by reference in their entireties. The polyvalent cation can be present in an amount effective to increase the melting temperature (Tₘ) of the phospholipid such that the pharmaceutical composition exhibits a Tₘ which is greater than its storage temperature (Tₘ) by at least about 20° C., such as at least about 40° C. The molar ratio of polyvalent cation to phospholipid can be at least about 0.05:1, such as about 0.05:1 to about 2.0:1 or about 0.25:1 to about 1.0:1. An example of the molar ratio of polyvalent cation:phospholipid is about 0.50:1. When the polyvalent cation is calcium, it can be in the form of calcium chloride. Although metal ion, such as calcium, is often included with phospholipid, none is required.

The pharmaceutical composition can include one or more surfactants. For instance, one or more surfactants can be in the liquid phase with one or more being associated with solid droplets or droplets of the composition. By “associated with” it is meant that the pharmaceutical compositions can incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and can be used to provide the desired preparations. In some aspects, the surfactant in the pharmaceutical composition described herein comprises Tween, sodium lauryl sulfate (SLS), or dipalmitoylphosphatidylcholine (DPPC). Without wishing to be bound to a certain theory, in some cases, the surfactant also serves as a solubility enhancer in the composition.

Examples of nonionic detergents include, but are not limited to, sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.), which is incorporated herein by reference in its entirety.

Examples of block copolymers include, but are not limited to, diblock and triblock copolymers of polyoxyethylene and polyoxypropylene, including poloxamer 188 (Pluronic™ F-68), poloxamer 407 (Pluronic™ F-127), and poloxamer 338. Examples of ionic surfactants include, but are not limited to, sodium sulfosuccinate, and fatty acid soaps. Examples of amino acids include, but are not limited to hydrophobic amino acids. Use of amino acids as pharmaceutically acceptable excipients is known in the art as disclosed in U.S. Pat. Nos. 6,123,936, 6,358,530, and 6,921,527, which are incorporated herein by reference in their entireties.

The pharmaceutical composition according to one or more embodiments of the disclosure may, if desired, contain a combination of pharmaceutical agent for treatment of pulmonary diseases (e.g., imatinib or a derivative thereof, e.g., imatinib free base or imatinib salt) and one or more additional active agents. Examples of additional active agents include, but are not limited to, agents that can be delivered through the lungs.

Additional active agents can comprise, for example, hypnotics and sedatives, psychic energizers, tranquilizers, respiratory drugs, anticonvulsants, muscle relaxants, antiparkinson agents (dopamine antagonists), analgesics, anti-inflammatories, antianxiety drugs (anxiolytics), appetite suppressants, antimigraine agents, muscle contractants, additional anti-infectives (antivirals, antifungals, vaccines) antiarthritics, antimalarials, antiemetics, antiepileptics, cytokines, growth factors, anti-cancer agents, antithrombotic agents, antihypertensives, cardiovascular drugs, antiarrhythmics, antioxidants, anti-asthma agents, hormonal agents including contraceptives, sympathomimetics, diuretics, lipid regulating agents, antiandrogenic agents, antiparasitic, anticoagulants, neoplastics, antineoplastics, hypoglycemics, nutritional agents and supplements, growth supplements, antienteritis agents, vaccines, antibodies, diagnostic agents, and contrasting agents. The additional active agent, when administered by inhalation, can act locally or systemically.

The additional active agent can fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of additional active agents suitable for use in this disclosure include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFFR) gene, deoxyribonuclease (DNase), bactericidal/permeability increasing protein (BPI), anti-CMV antibody, 13-cis retinoic acid, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem, pentamidine isethiouate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the disclosure is intended to encompass synthetic, native, glycosylated, unglycosylated, pegylated forms, and biologically active fragments, derivatives, and analogs thereof.

Additional active agents for use in the disclosure can further include nucleic acids, as bare nucleic acid molecules, vectors, associated viral droplets, plasmid DNA or RNA or other nucleic acid constructions of a type suitable for transfection or transformation of cells, e.g., suitable for gene therapy including antisense. Further, an active agent can comprise live attenuated or killed viruses suitable for use as vaccines. Other useful drugs include those listed within the Physician's Desk Reference (most recent edition), which is incorporated herein by reference in its entirety.

When a combination of active agents is used, the agents can be provided in combination in a single species of pharmaceutical composition or individually in separate species of pharmaceutical compositions.

The pharmaceutical compositions of one or more embodiments of the present disclosure can lack taste. In this regard, although taste masking agents are optionally included within the composition, the compositions in some embodiments do not include a taste masking agent other than a cyclodextrin and lack taste even without a taste masking agent.

In some embodiments, the pharmaceutical composition provided herein comprises a sweetener to improve the organoleptic properties of the composition. The sweetener can be a natural sweet substance, e.g. certain sugars, or an artificial sweetener. Without wishing to be bound to a certain theory, the presence of the sweetener in the pharmaceutical composition can improve the organoleptic properties of the composition. In some cases, the presence of the sweetener in the pharmaceutical composition can improve the compliance of the subject, presence of the sweetener in the pharmaceutical composition can increase the delivery efficiency of the composition. In some embodiments, the presence of the sweetener in the pharmaceutical composition can enhance the therapeutic effects of the composition.

Non-limiting examples of artificial sweeteners that can be used in the pharmaceutical composition include acesulfame potassium, aspartame, cyclamate, mogrosides, saccharin, stevia, sucralose, neotame, and sugar alcohols (e.g., erythritol, hydrogenated starch hydrolysates, isomalt, lactitol, maltitol, mannitol, sorbitol, and xylitol), such as those used in commercial products, like Sweet n′ low powder sweetener, Truvia powder sweetener, Equal (aspartame), Stevia powder sachet, Aspen Naturals liquid stevia, Now Better Stevia liquid sweetener, Sweet N′ Low liquid sweetener, Quick Sweet: Neotame liquid sweetener, or Splenda powder sachet, or pharmaceutically acceptable salts thereof. In some embodiments, the pharmaceutical composition comprises saccharin. In some embodiments, the pharmaceutical composition comprises a salt of saccharin. In some embodiments, the pharmaceutical composition comprises saccharin sodium.

Natural sweet substances that can be used in the pharmaceutical composition include, but not limited to, sucrose, agave, brown sugar, confectioner's (powdered) sugar, corn syrup, dextrose, fructose, fruit juice concentrate, glucose, high-fructose corn syrup, honey, invert sugar, lactose, malt sugar, maltose, maple syrup, molasses, nectars, raw sugar, and syrup. Sugars can increase the viscosity of the liquid solution, thus the concentration of any sugar added into the pharmaceutical composition, in some embodiments, is tightly controlled below a certain threshold value.

In some embodiments, pharmaceutically acceptable excipient or carrier comprises lactose, mannitol, sorbitol, erythritol, raffinose, sucrose, xylitol, trehalose, dextrose, cyclodextrins, maltitol, maltose, glucose, hydroxyapatite, or any combinations thereof.

Besides the above mentioned pharmaceutically acceptable excipients, it can be desirable to add other pharmaceutically acceptable excipients to the pharmaceutical composition to improve droplet rigidity, production yield, emitted dose and deposition, shelf-life, and patient acceptance. Such optional pharmaceutically acceptable excipients include, but are not limited to: coloring agents, taste masking agents, buffers, hygroscopic agents, antioxidants, and chemical stabilizers. In some embodiments, the compositions may include one or more osmolarity adjuster, such as sodium chloride. For instance, sodium chloride may be added to solutions to adjust the osmolarity of the solution.

In one or more embodiments, an aqueous pharmaceutical composition described herein comprises of essentially a pharmaceutical agent for treating pulmonary diseases (e.g., imatinib), water, pH buffer, solubility enhancer, and osmolarity adjuster. In some embodiments, the osmolarity adjuster can provide stability of aerosolized pharmaceutical composition, reduce adverse reaction to inhaled aerosolized pharmaceutical compositions (e.g., coughing), efficiency of aerosolization, or influence the droplet size. In some embodiments, the osmolarity of the aqueous pharmaceutical composition described herein are acceptable for pharmaceutical use (e.g., iso-osmolar, physiologic osmolarity, hypo-osmolar, physiologically hypotonic, hyper-osmolar, physiologically hypertonic). In some embodiments, the osmolarity of the aqueous pharmaceutical composition at about 0.001 mOsm to about 2,000 mOsm, for example between about 0.1 mOsm to about 1,000 mOsm, about 1 mOsm to about 200 mOsm, about 100 mOsm to about 200 mOsm, about 100 mOsm to about 500 mOsm, about 200 mOsm to about 400 mOsm, about 250 mOsm to about 350 mOsm, about 300 mOsm to about 400 mOsm, about 300 mOsm to about 2,000 mOsm, or about 1,000 mOsm to 2,000 mOsm. In some cases, the osmolality adjuster is a salt, such as sodium chloride or sodium carbonate.

### Nebulized Aerosol

Droplets

The distribution of aerosol droplets of an inhalable formulation can be expressed in terms of either: the mass median aerodynamic diameter (MMAD)—the size at which half of the mass of the aerosol is contained in smaller droplets and half in larger droplets; volumetric mean diameter (VMD); mass median diameter (MMD); the fine droplet fraction (FDF)—the percentage of droplets that are <5 um in diameter. These measures have been used for comparisons of the in vitro performance of different nebulizers and drug combinations. In general, the higher the fine droplet fraction, the higher the proportion of the emitted dose that is likely to deposit the lung. Generally, inhaled droplets are subject to deposition by one of two mechanisms: impaction, which usually predominates for larger droplets, and sedimentation, which is prevalent for smaller droplets. Impaction can occur when the momentum of an inhaled droplet is large enough that the droplet does not follow the air stream and encounters a physiological surface. In contrast, sedimentation can occur primarily in the deep lung when very small droplets which have traveled with the inhaled air stream encounter physiological surfaces as a result of random diffusion within the air stream.

For pulmonary administration, the upper airways are usually avoided in favor of the middle and lower airways. Pulmonary drug delivery can be accomplished by inhalation of an aerosol through the mouth and throat. Droplets having a mass median aerodynamic diameter (MMAD) of greater than about 5 microns generally do not reach the lung; instead, they tend to impact the back of the throat and are swallowed and possibly orally absorbed. Droplets having diameters of about 1 to about 5 microns are small enough to reach the upper-to-mid-pulmonary region (conducting airways), but are too large to reach the alveoli. Smaller droplets, i.e., about 0.5 to about 2 microns, are capable of reaching the alveolar region. Droplets having diameters smaller than about 0.5 microns can also be deposited in the alveolar region by sedimentation, although very small droplets can be exhaled. Measures of droplet size can be referred to as volumetric mean diameter (VMD), mass median diameter (MMD), or MMAD. These measurements can be made by impaction (MMD and MMAD) or by laser (VMD). For liquid particles, VMD, MMD and MMAD may be the same if environmental conditions are maintained, e.g., standard humidity. However, if humidity is not maintained, MMD and MMAD determinations will be smaller than VMD due to dehydration during impactor measurements. For the purposes of this description, VMD, MMD and MMAD measurements are considered to be under standard conditions such that descriptions of VMD, MMD and MMAD will be comparable.

Aerosol particle size may be expressed in terms of the mass median aerodynamic diameter (MMAD). Large droplets (e.g., MMAD-5 um) can deposit in the upper airway because they are too large to navigate the curvature of the upper airway. Small droplets (e.g., MMAD-2 um) can be poorly deposited in the lower airways and thus become exhaled, providing additional opportunity for upper airway deposition. Hence, intolerability (e.g., cough and bronchospasm) can occur from upper airway deposition from both inhalation impaction of large droplets and settling of small liquid droplets during repeated inhalation and expiration.

Thus, in one embodiment, an optimum droplet size is used (e.g., MMAD=1-5 um) in order to maximize deposition at a mid-lung and to minimize intolerability associated with upper airway deposition. Moreover, generation of a defined droplet size with limited geometric standard deviation (GSD) can optimize deposition and tolerability. Narrow GSD limits the number of droplets outside the desired MMAD size range. In one embodiment, an aerosol containing one or more compounds disclosed herein is provided having a MMAD from about 1 microns to about 5 microns with a GSD of less than or equal to about 2.5 microns. In another embodiment, an aerosol having an MMAD from about 2.8 microns to about 4.3 microns with a GSD less than or equal to 2 microns is provided. In another embodiment, an aerosol having an MMAD from about 2.5 microns to about 4.5 microns with a GSD less than or equal to 1.8 microns is provided.

In some embodiments, the nebulizer used in any of the methods described herein is a liquid nebulizer. In some embodiments, the nebulizer used in any of the methods described herein is a jet nebulizer, an ultrasonic nebulizer, a pulsating membrane nebulizer, a nebulizer comprising a vibrating mesh or plate with multiple apertures, or a nebulizer comprising a vibration generator and an aqueous chamber. In some embodiments, the nebulizer used in any of the methods described herein is a nebulizer comprising a vibrating mesh or plate with multiple apertures. In some embodiments, the liquid nebulizer achieves lung deposition of the imatinib or derivative thereof, or a salt thereof administered to the mammal; provides a Geometric Standard Deviation (GSD) of emitted droplet size distribution of the aqueous solution of about 1.0 um to about 2.5 um; provides a mass median aerodynamic diameter (MMAD) of droplet size of the aqueous solution emitted with the high efficiency liquid nebulizer of about 1 um to about Sum; a volumetric mean diameter (VMD) of about 1 um to about 5 um; and/or a mass median diameter (MMD) of about 1 um to about Sum; provides a fine droplet fraction (FDF=% 5 microns) of droplets emitted from the liquid nebulizer of at least about 30%; provides an output rate of at least 0.1 mL/min: and/or provides at least about 25% of the aqueous solution to the mammal.

In some embodiments, the composition (e.g., pharmaceutical composition or formulation) is aerosolized with nebulized droplets having an average mass median aerodynamic diameter of from 1 μm to 5 μm, from 1 μm to 4 μm, from 1 μm to 3 μm, from 1 μm to 2 μm, from 2 μm to 5) μm, from 2 μm to 4 μm, from 2 μm to 3 μm, or from 3 μm to 4 μm.

Nebulizer

In one embodiment, a nebulizer is selected on the basis of allowing the formation of an aerosol of the composition described herein. In some embodiments, the MMAD of nebulized or aerosolized composition has a predominately MMAD of between about 1 to about 5 microns.

Efficient drug delivery to the lungs through nebulizers is dependent on several factors including inhaler device, formulation, and inhalation maneuver. The pharmaceutical compositions can be administered using an aerosolization device. The aerosolization device can be a nebulizer, a metered dose inhaler, or a liquid dose instillation device. The aerosolization device can comprise the extrusion of the pharmaceutical preparation through micron or submicron-sized holes with subsequent Rayleigh break-up into fine droplets. The pharmaceutical composition can be delivered by a nebulizer as described in WO 99/16420, by a metered dose inhaler as described in WO 99/16422, by a liquid dose instillation apparatus as described in WO 99/16421, which are incorporated herein by reference in their entireties. As such, an inhaler can comprise a canister containing the droplets or droplets and propellant, and wherein the inhaler comprises a metering valve in communication with an interior of the canister. The propellant can be a hydrofluoroalkane.

For instance, the pharmaceutical composition can be in liquid solution, and can be administered with nebulizers, such as that disclosed in WO 99/16420, the disclosure of which is hereby incorporated in its entirety by reference, in order to provide an aerosolized medicament that can be administered to the pulmonary air passages of a patient in need thereof. Nebulizers known in the art can easily be employed for administration of the claimed formulations. Breath-activated or breath-actuated nebulizers, as well as those comprising other types of improvements which have been, or will be, developed are also compatible with the formulations of the present disclosure and are contemplated as being within the scope thereof.

In some cases, the nebulizer is a breath activated or breath-actuated nebulizer. In some cases, the nebulizer is a hand-held inhaler device (e.g., AeroEclipse® Breath Actuated Nebulizer (BAN)). In some cases, the nebulizer has a compressed air source. In some cases, the nebulizer converts liquid medication into an aerosol. In some cases, the nebulizer converts liquid medication into an aerosol by extruding the pharmaceutical preparation through micron or submicron-sized holes. In some cases, the nebulizer converts liquid medication into an aerosol so it can be inhaled into the lungs. In some cases, the nebulizer is a small volume nebulizer. In some cases, the nebulizer is a small volume jet nebulizer. In some cases, aerosolized medication is only produced when inhaled through the device. In some cases, the medication is contained in the cup between breaths or during breaks in treatment. In some cases, the medication is contained in the cup until ready to be inhaled.

Nebulizers can impart energy into a liquid composition to aerosolize the liquid, and to allow delivery to the pulmonary system, e.g., the lungs, of a patient. A nebulizer comprises a liquid delivery system, such as a container having a reservoir that contains a liquid composition. The liquid composition generally comprises an active agent that is either in solution or suspended within a liquid medium.

In one type of nebulizer that can be used in the subject methods and kits, generally referred to as a jet nebulizer, compressed gas is forced through an orifice in the container. The compressed gas forces liquid to be withdrawn through a nozzle, and the withdrawn liquid can mix with the flowing gas to form aerosol droplets. A cloud of droplets can then be administered to the patients respiratory tract. In another type of nebulizer that can be used in the subject methods and kits, generally referred to as a vibrating mesh nebulizer, energy, such as mechanical energy, vibrates a mesh. This vibration of the mesh aerosolizes the liquid composition to create an aerosol cloud that is administered to the patient's lungs. In another type of nebulizer that can be used in the subject methods and kits, the nebulizing comprises extrusion through micron or submicron-sized holes followed by Rayleigh break-up into fine droplets. Alternatively or additionally, the composition may be in a liquid form and may be aerosolized using a nebulizer as described in WO 2004/071368, which is herein incorporated by reference in its entirety, as well as U.S. Published application Nos. 2004/0011358 and 2004/0035413, which are both herein incorporated by reference in their entireties. Other examples of nebulizers include, but are not limited to, the Aeroneb® Go or Aeroneb® Pro nebulizers, available from Aerogen Ltd. of Galway, Ireland; the PART eFlow and other PARI nebulizers available from PARI Respiratory Equipment, Inc. of Midlothian, Va.; the Lumiscope® Nebulizer 6600 or 6610 available from Lumiscope Company, Inc. of East Brunswick, N.J.; and the Omron NE-U22 available from Omron Healthcare, Inc. of Kyoto, Japan. Other examples of nebulizers include devices produced by Medspray (Enschede, The Netherlands) and Pulmotree Medical GmbH (Munchen, Germany).

A nebulizer of the vibrating mesh type, such as one that that forms droplets without the use of compressed gas, such as the Aeroneb® Pro can provide unexpected improvement in dosing efficiency and consistency. By generating fine droplets by using a vibrating perforated or unperforated membrane, rather than by introducing compressed air, the aerosolized composition can be introduced without substantially affecting the flow characteristics. In addition, the generated droplets when using a nebulizer of this type can be introduced at a low velocity, thereby decreasing the likelihood of the droplets being driven to an undesired region. When using a nebulizer of the extrusion/Rayleigh jet breakup type, the generated droplets can also be introduced at a low velocity, thereby decreasing the likelihood of the droplets being driven to an undesired region.

In some cases, the nebulizer that can be used in the subject methods and kits is of the vibrating mesh type. In some cases, the nebulizer that can be used in the subject methods and kits is of the pressurized jet type. In some cases, the nebulizer that can be used in the subject methods and kits is of the extrusion/Rayleigh breakup type. In some cases, the nebulizer is lightweight (at most 60 g, at most 100 g, at most 200 g, at most 250 g) and nearly silent. In some cases, the nebulizer has a sound level less than 35 A-weighted decibels (dBA) at 1 meter. In some cases, the nebulizer has a medication cup capacity of 6 mL. In some cases, the nebulizer has a residual volume of less than 0.3 mL. In some cases, the nebulizer generates an average flow rate of 0.4 mL/min. In some cases, the nebulizer generates an average flow rate of 0.5 mL/min. In some cases, the nebulizer generates an average flow rate of 0.6 mL/min. In some cases, the nebulizer generates an average flow rate of 0.7 mL/min. In some cases, the nebulizer generates an average flow rate of 0.8 mL/min. In some cases, the nebulizer generates an average flow rate of 0.9 mL/min. In some cases, the nebulizer generates an average flow rate of 1.0 mL/min. In some cases, the nebulizer generates an average flow rate of 1.1 mL/min. In some cases, the nebulizer generates an average flow rate of 1.2 mL/min. In some cases, the nebulizer generates an average droplet size of 3.0 μm MMAD. In some cases, the nebulizer generates an average droplet size between 3.0 μm MMAD and 4.0 μm MMAD. In some cases, the nebulizer generates an average droplet size of 3.0 μm MMAD. In some cases, the nebulizer generates an average droplet size between 3.0 μm MMAD and 5.0 μm MMAD. In some cases, the nebulizer generates an average droplet size of 3.0 μm MMAD. In some cases, the nebulizer generates an average droplet size between 3.0 μm MMAD and 6.0 μm MMAD. In still another type of nebulizer that can be used in the subject methods and kits, ultrasonic waves are generated to directly vibrate and aerosolize the composition. The compositions disclosed herein can also be administered to the lungs of a patient via aerosolization, such as with a metered dose inhaler. The use of such formulations provides for superior dose reproducibility and improved lung deposition as disclosed in WO99/16422, hereby incorporated in its entirety by reference. Metered dose inhalers (MDIs) known in the art can be employed for administration of the claimed compositions. Breath-activated or breath-actuated MDIs and pressurized MDIs (pMDIs), as well as those comprising other types of improvements which have been, or will be, developed are also compatible with the formulations of the present disclosure and, as such, are contemplated as being within the scope thereof. Along with MDIs and nebulizers, it will be appreciated that the formulations of one or more embodiments of the present disclosure can be used in conjunction with liquid dose instillation or LDI techniques as disclosed in, for example, WO 99/16421, which is incorporated herein by reference in its entirety. Liquid dose instillation involves the direct administration of a formulation to the lung. With respect to LDI the formulations are preferably used in conjunction with partial liquid ventilation or total liquid ventilation. Moreover, one or more embodiments of the present disclosure may further comprise introducing a therapeutically beneficial amount of a physiologically acceptable gas (such as nitric oxide or oxygen) into the pharmaceutical microdispersion prior to, during or following administration.

Aqueous formulations may be aerosolized by liquid nebulizers employing either hydraulic or ultrasonic atomization. Propellant-based systems may use suitable pressurized metered-dose inhalers (pMDIs). A desired particle size and distribution may be obtained by choosing an appropriate device. In some embodiments, the nebulizer is a jet nebulizer, a vibrating mesh nebulizer, or an ultrasonic nebulizer.

Ready-to-Use Formulation

The pharmaceutical compositions provided herein is ready-to-use for treatment of pulmonary diseases for immediate use. In contrast, other drug formulations comprising imatinib are in powder form for liquid suspension for oral use or in concentrated liquid form/stock solution that require dilution prior to usage necessitate additional components and steps. The pharmaceutical compositions described herein are in aqueous liquid form that provides immediate usage for at moment a pulmonary disease related symptom without need of additional steps. The ready-to-use formulation of the pharmaceutical compositions described herein invasive procedures such as intravenous administration.

In some embodiments, the ready-to-use pharmaceutical composition is in a single use dose to treat a pulmonary disease or symptom of a pulmonary disease. In some embodiments, the single use dosage is in a cartridge or container with a volume for the single usage. In some embodiments, the single use dosage is in a cartridge or container with a volume for additional application of the pharmaceutical composition. In some embodiments, the ready-to-use pharmaceutical composition is in a multiple dosage formulation. In some embodiments, the multiple dosage formulation of the ready-to-use pharmaceutical formulation is in a cartridge or container wherein the volume contains the multiple dosages. In some embodiments, the multiple dosage formulations require refilling a cartridge or container of the ready-to-use pharmaceutical composition for nebulization. In some embodiments, a measuring tool and/or transfer tool is included in kits for refilling a cartridge or container for multiple dosage usage.

### Methods of Treatment

Pulmonary Diseases

The methods, compositions, and kits provided herein can include administration of the pharmaceutical composition via inhalation, e.g., oral or nasal inhalation. Examples of pulmonary diseases can include, but not limited to, asthma, emphysema, chronic obstructive pulmonary disease (COPD), infections (e.g., pneumonia, tuberculosis, influenza), coccidioidomycosis, corona virus, cytogenetic organizing pneumonia (COP), pulmonary arterial hypertension, respiratory syncytial virus (RSV), hantavirus pulmonary syndrome (HPS), Mycobacterium avium complex lung disease, Middle Eastern Respiratory Syndrome (MERS), mesothelioma, nontuberculous mycobacteria lung disease (NTM), severe acute respiratory syndrome (SARS), lung cancer, acute respiratory distress syndrome (ARDS), alpha-1 antitrypsin deficiency (AAT), asbestosis, aspergillosis, bronchiectasis, bronchiolitis, bronchiolitis obliterans, acute bronchitis, bronchopulmonary dysplasia, chronic bronchitis, chronic cough, Coal Worker's pneumoconiosis (Black Lung Disease), cystic fibrosis, e-cigarette or vaping use-associated lung injury (EVALI), eosinophilic granulomatosis with polyangiitis, histoplasmosis, human metapneumovirus (hMPV), hypersensitivity pneumonitis, idiopathic pulmonary fibrosis (IPF), institial lung disease (ILD), sarcoidosis, Legionnaires' disease, pertussis (whooping cough), primary ciliary dyskinesia (PCT), pulmonary arterial hypertension (PAH), silicosis, or pulmonary fibrosis. The pharmaceutical compositions described herein can be used to treat diseases and symptoms relating to a lung disorder, respiratory diseases, and trachea or bronci disorders. In some cases, a lung disorder may arise from inhalation and/or exposure to tobacco smoke, infections (e.g., bacterial, viral, fungal), radon, asbestos, air pollution, particulates, debris. In some cases, a lung disorder or symptom of the lung disorder may arise from lung damage, sleep apnea, collapsed lung, or pulmonary embolism.

Thus, the pharmaceutical compositions according to some examples of the present disclosure can be used to treat and/or provide prophylaxis for a broad range of patients. A suitable patient for, receiving treatment and/or prophylaxis as described herein is any mammalian patient in need thereof, preferably such mammal is a human. Examples of subjects include, but are not limited to, pediatric patients, adult patients, and geriatric patients. In some cases, the composition is intended only as a treatment for rapid resolution of symptoms and restoration of normal sinus rhythm, and is not taken as a preventative, e.g., when the patient is well, there is no need for drug—this can increase the benefit-risk ratio of the therapy and overall safety due to the sporadic or intermittent dosing, and the focus on reducing disabling symptoms and restoring sinus rhythm only when needed.

Pharmaceutical compositions disclosed herein can be more effective in subjects that include or lack certain physiological or demographic factors, such as, for example, age at clinical presentation, certain hemodynamic criteria, electrophysiological features, and prior treatments. In some embodiments, a subject treated with a pharmaceutical composition of the disclosure suffers from a pulmonary disease with an onset that occurred within 48 hours prior to the treating. In some embodiments, a subject treated with a pharmaceutical composition of the disclosure suffers from a pulmonary disease with an onset that occurred from 1 hour to 48 hours prior to the treating. In some embodiments, a subject treated with a pharmaceutical composition of the disclosure suffers from recurrent pulmonary disease. In some embodiments, a subject treated with a pharmaceutical composition of the disclosure has an ongoing prescription medication for a pulmonary disease. In some embodiments, the oral medication for treating the pulmonary disease is imatinib, or a pharmaceutically acceptable salt thereof.

In some embodiments, a subject treated with a pharmaceutical composition of the disclosure is over 18 years in age. In some embodiments, a subject treated with a pharmaceutical composition of the disclosure is under the age of 18. In some embodiments, a subject treated with a pharmaceutical composition of the disclosure is no more than 85 years in age. In some embodiments, a subject treated with a pharmaceutical composition of the disclosure is from 18 years old to 85 years old.

In some embodiments, a subject treated with a pharmaceutical composition of the disclosure does not exhibit severe renal impairment, wherein a eGFR of the subject is less than 30 mL/min/1.73 m² at the time of treating. In some embodiments, a subject treated with a pharmaceutical composition of the disclosure is not on dialysis at the time of treating. In some embodiments, a subject treated with a pharmaceutical composition of the disclosure does not exhibit abnormal liver function at the time of treating. In some embodiments, the abnormal liver function is hepatic disease or biochemical evidence of significant liver derangement. In some embodiments, a subject treated with a pharmaceutical composition of the disclosure does not exhibit uncorrected hypokalemia at the time of treating. In some embodiments, a subject treated with a pharmaceutical composition of the disclosure does not exhibit a serum potassium less than 3.6 mEq/L at the time of treating.

In some embodiments, a subject treated with a pharmaceutical composition of the disclosure does not exhibit an established pulmonary disease in need of inhalation medication at the time of treating. In some embodiments, a subject treated with a pharmaceutical composition of the disclosure does not have a hypersensitivity to imatinib or any of its active metabolites, or a history thereof. In some embodiments, a subject treated with a pharmaceutical composition of the disclosure is not concomitantly administered a systemic drug that is an inhibitor of CYP 2D6. In some embodiments, the inhibitor of CYP 2D6 is an antidepressant, a neuroleptic, or an antihistamine. In some embodiments, the inhibitor of CYP 2D6 is propranolol or ritonavir. In some embodiments, a subject treated with a pharmaceutical composition of the disclosure is not concomitantly administered a systemic drug that is a CYP 2D6 inducer. In some embodiments, the CYP 2D6 inducer is phenytoin, phenobarbital, or carbamazepine.

In some embodiments, a subject treated with a pharmaceutical composition of the disclosure does not exhibit a congenital lung disease at the time of treating. In some embodiments, a subject treated with a pharmaceutical composition of the disclosure does not exhibit syncope at the time of treating.

In some embodiments, a subject treated with a pharmaceutical composition of the disclosure does not exhibit any serious or life threatening medical condition other than a pulmonary disease symptoms at the time of treating. In some embodiments, a subject treated with a pharmaceutical composition of the disclosure does not exhibit an acute pathogenic infection at the time of treating.

In some embodiments, a subject treated with a pharmaceutical composition of the disclosure has not exhibited a drug or alcohol dependence within 12 months prior to administration of the pharmaceutical composition. In some embodiments, a subject treated with a pharmaceutical composition of the disclosure does not exhibit a body mass index greater than 40 Kg/m² at the time of treating.

The therapy provided herein can comprise or be suitable for inhalation, e.g., oral or nasal inhalation. In some cases, during administration via oral inhalation, the pharmaceutical agent is inhaled by the patient through the mouth and absorbed by the lungs. In some cases, during administration via nasal inhalation, the pharmaceutical agent is inhaled by the patient through the nose and absorbed by the nasal mucous and/or the lungs.

The inhalation route can avoid first-pass hepatic metabolism, hence dosing variability can be eliminated. Unlike the case for oral tablets or pills, the patient's metabolic rates may not matter as the administration is independent of the metabolic paths experienced when a drug is administered via oral route through gastrointestinal tract, e.g., as tablets, pills, solution, or suspension. A fast onset of action, a potential improvement in efficacy, and/or a reduction in dose can be achieved with the fast absorption of drugs from the nasal mucosa and/or lungs.

The fast absorption rate of drugs through the lungs can be achieved because of the large surface area available in the lungs for aerosols small enough to penetrate central and peripheral lung regions. Consequently, the rate and extent of absorption of drugs delivered via inhalation can yield plasma concentrations vs. time profiles that are comparable with the IV route of administration.

In some cases, the therapy provided herein is provided to a subject for more than once on an as-needed basis. For instance, the therapy can be administered to a subject, e.g., the pharmaceutical composition is inhaled by the subject, for at least once per day, e.g., for 1, 2, 3, 4, 5, 6, 8, or 10 times per day. In some cases, the therapy can be administered to a subject for an extended period of time, for instance, for a period of at least 5, 10, 20, 30, 60, 100, or 300 days, at least 1, 2, 3, 4, or 5 years, during which time the subject receives administration of the therapy on a daily basis, or every other day, or every 2, 3, 4, 5, 6, 7, or 10 days. On each day that the therapy is administered to the subject, the subject can receive administration of the therapy, e.g., inhale the pharmaceutical composition provided herein, for at least once, e.g., 1, 2, 3, 4, 5, 6, 8, or 10 times.

Dosage

The pharmaceutical composition can be administered to the patient on an as-needed basis.

In some embodiments, the unit dosage is about 20 mg to about 500 mg of the pharmaceutical agent for treating pulmonary diseases. In some embodiments, the unit dosage is about 20 mg to about 500 mg of the pharmaceutical agent for treating pulmonary diseases wherein the pharmaceutical agent for treating pulmonary diseases is imatinib or a pharmaceutically acceptable salt thereof. In some embodiments, the unit dosage is about 30 mg to about 500 mg of the pharmaceutical agent for treating pulmonary diseases. In some embodiments, the unit dosage is about 40 mg to about 500 mg of the pharmaceutical agent for treating pulmonary diseases. In some embodiments, the unit dosage is about 50 mg to about 500 mg of the pharmaceutical agent for treating pulmonary diseases. In some embodiments, the unit dosage is about 100 mg to about 500 mg of the pharmaceutical agent for treating pulmonary diseases. In some embodiments, the unit dosage is about 120 mg to about 500 mg of the pharmaceutical agent for treating pulmonary diseases. In some embodiments, the unit dosage is about 150 mg to about 500 mg of the pharmaceutical agent for treating pulmonary diseases. In some embodiments, the unit dosage is about 200 mg to about 500 mg of the pharmaceutical agent for treating pulmonary diseases. In some embodiments, the unit dosage is about 250 mg to about 500 mg of the pharmaceutical agent for treating pulmonary diseases. In some embodiments, the unit dosage is about 300 mg to about 500 mg of the pharmaceutical agent for treating pulmonary diseases. In some embodiments, the unit dosage is about 350 mg to about 500 mg of the pharmaceutical agent for treating pulmonary diseases. In some embodiments, the unit dosage is about 400 mg to about 500 mg of the pharmaceutical agent for treating pulmonary diseases. In some embodiments, the unit dosage is about 450 mg to about 500 mg of the pharmaceutical agent for treating pulmonary diseases. In some embodiments, the unit dosage is about 30 mg to about 500 mg of the pharmaceutical agent for treating pulmonary diseases. In some embodiments, the unit dosage is about 20 mg to about 450 mg of the pharmaceutical agent for treating pulmonary diseases. In some embodiments, the unit dosage is about 20 mg to about 400 mg of the pharmaceutical agent for treating pulmonary diseases. In some embodiments, the unit dosage is about 20 mg to about 350 mg of the pharmaceutical agent for treating pulmonary diseases. In some embodiments, the unit dosage is about 20 mg to about 300 mg of the pharmaceutical agent for treating pulmonary diseases. In some embodiments, the unit dosage is about 20 mg to about 250 mg of the pharmaceutical agent for treating pulmonary diseases. In some embodiments, the unit dosage is about 20 mg to about 200 mg of the pharmaceutical agent for treating pulmonary diseases. In some embodiments, the unit dosage is about 20 mg to about 150 mg of the pharmaceutical agent for treating pulmonary diseases. In some embodiments, the unit dosage is about 20 mg to about 120 mg of the pharmaceutical agent for treating pulmonary diseases. In some embodiments, the unit dosage is about 20 mg to about 100 mg of the pharmaceutical agent for treating pulmonary diseases. In some embodiments, the unit dosage is about 20 mg to about 50 mg of the pharmaceutical agent for treating pulmonary diseases. In some embodiments, the unit dosage is about 20 mg to about 40 mg of the pharmaceutical agent for treating pulmonary diseases. In some embodiments, the unit dosage is about 20 mg to about 30 mg of the pharmaceutical agent for treating pulmonary diseases. In some embodiments, the unit dosage is about 30 mg to about 450 mg of the pharmaceutical agent for treating pulmonary diseases. In some embodiments, the unit dosage is about 40 mg to about 400 mg of the pharmaceutical agent for treating pulmonary diseases. In some embodiments, the unit dosage is about 50 mg to about 350 mg of the pharmaceutical agent for treating pulmonary diseases. In some embodiments, the unit dosage is about 100 mg to about 300 mg of the pharmaceutical agent for treating pulmonary diseases. In some embodiments, the unit dosage is about 150 mg to about 250 mg of the pharmaceutical agent for treating pulmonary diseases. In some embodiments, the unit dosage is about 100 mg to about 150 mg of the pharmaceutical agent for treating pulmonary diseases. In some embodiments, the unit dosage is about 20 mg to about 200 mg of the pharmaceutical agent for treating pulmonary diseases. In some embodiments, the unit dosage is about 200 mg to about 500 mg of the pharmaceutical agent for treating pulmonary diseases. In some embodiments, the unit dosage is about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg of the pharmaceutical agent for treating pulmonary diseases.

In some cases, the unit dose of the pharmaceutical composition described herein in aqueous solution is from 20 mg/mL to 500 mg/mL of imatinib. In some cases, the unit dose is 20 to 500 mg/mL, from 20 to 450 mg/mL, from 20 to 400 mg/mL, from 20 to 350 mg/mL, from 20 to 300 mg/mL, from 20 to 250 mg/mL, from 20 to 200 mg/mL, from 20 to 150 mg/mL, from 20 to 120 mg/mL, from 20 to 100 mg/mL, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 40 mg/mL to 200 mg/mL, from 40 mg/mL to 250 mg/mL, from 40 mg/mL to 300 mg/mL, from 40 mg/mL to 350 mg/mL, from 40 mg/mL to 400 mg/mL, from 40 mg/mL to 450 mg/mL, from 40 mg/mL to 500 mg/mL, from 60 mg/mL to 80 mg/mL, from 60 mg/mL to 100 mg/mL, from 60 mg/mL to 120 mg/mL, from 60 mg/mL to 150 mg/mL, from 60 mg/mL to 200 mg/mL, from 60 mg/mL to 250 mg/mL, from 60 mg/mL to 300 mg/mL, from 60 mg/mL to 350 mg/mL, from 60 mg/mL to 400 mg/mL, from 60 mg/mL to 450 mg/mL, from 60 mg/mL to 500 mg/mL, from 80 mg/mL to 100 mg/mL, from 80 mg/mL to 120 mg/mL, from 80 mg/mL to 150 mg/mL, from 80 mg/mL to 200 mg/mL, from 80 mg/mL to 250 mg/mL, from 80 mg/mL to 300 mg/mL, from 80 mg/mL to 350 mg/mL, from 80 mg/mL to 400 mg/mL, from 80 mg/mL to 450 mg/mL, from 80 mg/mL to 500 mg/mL, from 100 mg/mL to 120 mg/mL, from 100 mg/mL to 150 mg/mL, from 100 mg/mL to 200 mg/mL, from 100 mg/mL to 250 mg/mL, from 100 mg/mL to 300 mg/mL, from 100 mg/mL to 350 mg/mL, from 100 mg/mL to 400 mg/mL, from 100 mg/mL to 450 mg/mL, from 100 mg/mL to 500 mg/mL, from 120 mg/mL to 150 mg/mL, from 120 mg/mL to 200 mg/mL, from 120 mg/mL to 250 mg/mL, from 120 mg/mL to 300 mg/mL, from 120 mg/mL to 350 mg/mL, from 120 mg/mL to 400 mg/mL, from 120 mg/mL to 450 mg/mL, from 120 mg/mL to 500 mg/mL, from 150 mg/mL to 200 mg/mL, from 150 mg/mL to 250 mg/mL, from 150 mg/mL to 300 mg/mL, from 150 mg/mL to 350 mg/mL, from 150 mg/mL to 400 mg/mL, from 150 mg/mL to 450 mg/mL, from 150 mg/mL to 500 mg/mL, from 200 mg/mL to 250 mg/mL, from 200 mg/mL to 300 mg/mL, from 200 mg/mL to 350 mg/mL, from 200 mg/mL to 400 mg/mL, from 200 mg/mL to 450 mg/mL, from 200 mg/mL to 500 mg/mL, from 250 mg/mL to 300 mg/mL, from 250 mg/mL to 350 mg/mL, from 250 mg/mL to 400 mg/mL, from 250 mg/mL to 450 mg/mL, from 250 mg/mL to 500 mg/mL, from 300 mg/mL to 350 mg/mL, from 300 mg/mL to 400 mg/mL, from 300 mg/mL to 450 mg/mL, from 300 mg/mL to 500 mg/mL from 350 mg/mL to 400 mg/mL, from 350 mg/mL to 450 mg/mL, from 350 mg/mL to 500 mg/mL, from 400 mg/mL to 450 mg/mL, from 400 mg/mL to 500 mg/mL, mg/mL, or from 450 mg/mL to 500 mg/mL of imatinib.

The pharmaceutical composition of one or more embodiments of the present disclosure can have improved emitted dose efficiency. The emitted dose (ED) of the aerosolized liquid droplets of the present disclosure can be greater than about 30%, such as greater than about 40%, greater than about 50%, greater than about 60%, or greater than about 70%. The dose of the pharmaceutical agent for treatment of pulmonary diseases (e.g., imatinib free base, imatinib salt, imatinib mesylate, imatinib derivative) can be administered during a single inhalation or can be administered during several inhalations. The fluctuations of the pharmaceutical agent can be reduced by administering the pharmaceutical composition more often or can be increased by administering the pharmaceutical composition less often. Therefore, the pharmaceutical composition provided herein can be administered from about four times daily to about once a month, such as about once daily to about once every two weeks, about once every two days to about once a week, and about once per week.

In some cases, the pharmaceutical agent for treatment of pulmonary diseases is delivered over two or more inhalations. In some cases, time between the two or more inhalations is from about 0.1 to 10 minutes. The pharmaceutical agent for treatment of pulmonary diseases is administered in the described dose in less than 60 minutes, less than 50 minutes, less than 40 minutes, less than 30 minutes, less than 20 minutes, less than 15 minutes, less than 10 minutes, less than 7 minutes, less than 5 minutes, in less than 3 minutes, in less than 2 minutes, or in less than 1 minute. In some cases, delivery of the required dose of pharmaceutical agent for treating pulmonary diseases (e.g., imatinib) is completed with 1, 2, 3, 4, 5, or 6 inhalations. In some cases, each inhalation is performed for about 0.5, 1, 1.2, 1.5, 1.8, 2, 2.2, 2.5, 2.8, 3, 3.2, 3.5, 3.8, 4, 4.2, 4.5, 4.8, or 5 minutes. In some cases, each inhalation is performed for longer than 5 minutes. In some cases, each inhalation is performed for up to 4.5 minutes. In some cases, each inhalation comprises at least 60 inhalation breaths, 50 inhalation breaths, 40 inhalation breaths, 30 inhalation breaths, 20 inhalation breaths, 10 inhalation breaths, 8 inhalation breaths, 6 inhalation breaths, 4 inhalation breaths, 3 inhalation breaths, 2 inhalation breaths or 1 inhalation breath. In some cases, each inhalation comprises no more than 100 inhalation breaths, 90 inhalation breaths, 80 inhalation breaths, 70 inhalation breaths, 60 inhalation breaths, 50 inhalation breaths, 40 inhalation breaths, 30 inhalation breaths, or 20 inhalation breaths. In some cases, inhalation of the antiarrhythmic pharmaceutical agent is performed with deep lung breath that lasts for longer than 1 second, 2 seconds, 3 seconds, or 4 seconds. In some cases, inhalation of the pharmaceutical agent for treatment of pulmonary diseases is performed with deep lung breath that lasts for about 1 second, 2 seconds, 3 seconds, or 4 seconds.

In some embodiments, during inhalational delivery of the pharmaceutical agent for treatment of pulmonary diseases, the subject takes, or is instructed to take, a break between two inhalations. In such embodiments, the break between two inhalations lasts for about 0.1 to 10 minutes, such as, 0.2 to 5, 1 to 5, 1.5 to 5, 2 to 5, 3 to 5, 4 to 5, 1 to 1.5, 1 to 2, 1 to 2.5, 1 to 3, 1 to 3.5, 1 to 4, 1.5 to 2, 1.5 to 2.5, or 1.5 to 3 minutes. In some cases, the subject takes, or is instructed to take, a break for about 1 minute between two inhalations. In some cases, the inhalation pattern for delivery of a single dose goes as follows: a first inhalation for about 4 to 4.5 minutes, a break for about 1 minute, and a second inhalation for about 4 to 4.5 minutes; a first inhalation for about 4 to 4.5 minutes, a break for about 30 seconds, and a second inhalation for about 4 to 4.5 minutes; a first inhalation for about 4 to 4.5 minutes, a first break for about 1 minute, and a second inhalation for about 4 to 4.5 minutes; a second break for about 1 minutes, and a third inhalation for about 4 to 4.5 minutes; or a first inhalation for about 4 to 4.5 minutes, a first break for about 30 seconds, and a second inhalation for about 4 to 4.5 minutes; a second break for about 30 seconds, and a third inhalation for about 4 to 4.5 minutes.

In one version, the pharmaceutical composition described herein can be administered daily. In this case, the daily dosage of the imatinib ranges from about 0.1 mg to about 600 mg, such as about 0.5 mg to about 500 mg, about 1 mg to about 400 mg, about 2 mg to about 300 mg, and about 3 mg to about 200 mg.

In some cases, the therapy provided herein is provided to a subject for more than once on an as-needed basis. For instance, the present disclosure can involve a follow-up inhalation if symptoms of the pulmonary disease have not subsided and occurs after an initial inhalation. In some instances, if symptoms of the pulmonary disease have not subsided within 30 minutes of the initial inhalation, the follow-up dosage is higher or the same as the initial dosage.

In another version, the pharmaceutical composition is administered prophylactically to a subject who is likely to develop a pulmonary disease. For example, a patient who has a history of pulmonary diseases can be prophylactically treated with a pharmaceutical composition comprising imatinib to reduce the likelihood of developing a pulmonary disease.

The pharmaceutical composition can be administered to a patient in any regimen which is effective to prevent a pulmonary disease. Illustrative prophylactic regimes include administering a pharmaceutical agent for treating pulmonary diseases as described herein 1 to 21 times per week.

The amount of the imatinib that is delivered to the subject (e.g., approximately the amount of the imatinib free base exiting a mouthpiece when being inhaled by the subject) for the treatment of a pulmonary disease can be from about 20 mg to about 500 mg, such as 20 mg to 30 mg, 20 mg to 40 mg, 20 mg to 50 mg, 20 mg to 60 mg, 20 mg to 70 mg, 20 mg to 80 mg, 20 mg to 90 mg, 20 mg to 100 mg, 20 mg to 110 mg, 20 mg to 120 mg, 20 mg to 130 mg, 20 mg to 140 mg, 20 mg to 150 mg, 20 mg to 160 mg, 20 mg to 170 mg, 20 mg to 180 mg, 20 mg to 200 mg, 20 mg to 250 mg, 20 mg to 300 mg, 20 mg to 350 mg, 20 mg to 400 mg, 20 mg to 500 mg, 50 mg to 60 mg, 50 mg to 70 mg, 50 mg to 80 mg, 50 mg to 90 mg, 50 mg to 100 mg, 50 mg to 120 mg, 50 mg to 150 mg, 50 mg to 200 mg, 50 mg to 250 mg, 50 mg to 300 mg, 50 mg to 350 mg, 50 mg to 400 mg, 50 mg to 450 mg, 50 mg to 500 mg, 100 mg to 120 mg, 100 mg to 150 mg, 100 mg to 200 mg, 100 mg to 250 mg, 100 mg to 300 mg, 100 mg to 350 mg, 100 mg to 400 mg, 100 mg to 450 mg, 100 mg to 500 mg, 150 mg to 200 mg, 150 mg to 250 mg, 150 mg to 300 mg, 150 mg to 350 mg, 150 mg to 400 mg, 150 mg to 450 mg, 150 mg to 500 mg, 200 mg to 250 mg, 200 mg to 300 mg, 200 mg to 350 mg, 200 mg to 400 mg, 200 mg to 450 mg, 200 mg to 500 mg, 40 mg to 150 mg, 50 mg to 150 mg, 60 mg to 150 mg, 70 mg to 150 mg, 80 mg to 150 mg, 90 mg to 150 mg, 100 mg to 150 mg, 110 mg to 150 mg, 120 mg to 150 mg, 130 mg to 150 mg, 140 mg to 150 mg, 30 mg to 140 mg, 40 mg to 130 mg, 50 mg to 120 mg, 60 mg to 110 mg, 70 mg to 110 mg, or 80 mg to 100 mg.

In one version, the amount of the imatinib that is delivered to the subject (e.g., approximately the amount of the imatinib exiting the aerosolization device when being inhaled by the subject) for the treatment of pulmonary diseases, is at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 470 mg, or at least about 500 mg.

In one version, the amount of the imatinib that is delivered to the subject (e.g., approximately the amount of the imatinib exiting a mouthpiece when being inhaled by the subject) for the treatment of pulmonary diseases is at most about 20 mg, at most about 30 mg, at most about 40 mg, at most about 50 mg, at most about 60 mg, at most about 70 mg, at most about 80 mg, at most about 90 mg, at most about 100 mg, at most about 110 mg, at most about 120 mg, at most about 130 mg, at most about 140 mg, at most about 150 mg, at most about 160 mg, at most about 170 mg, at most about 180 mg, at most about 190 mg, at most about 200 mg, at most about 225 mg, at most about 250 mg, at most about 275 mg, at most about 300 mg, at most about 325 mg, at most about 350 mg, at most about 375 mg, at most about 400 mg, at most about 425 mg, at most about 450 mg, at most about 475 mg, or at most about 500 mg.

In some cases, the amount of the imatinib that is delivered to the subject (e.g., approximately the amount of the imatinib exiting a mouthpiece when being inhaled by the subject) for the treatment of pulmonary diseases is about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

Kits and Systems

In one aspect, also provided herein are kits for treatment of pulmonary diseases via inhalation. The kits can include one or more pharmaceutical agents, for instance, a salt of imatinib, or some additional active agent(s) as described herein. In some cases, the kits include container for the pharmaceutical agents or compositions. In some cases, unit doses of the pharmaceutical agents as discussed above are provided in the kits. In some cases, the kits also include containers/receptacles for containing the pharmaceutical agents.

The pharmaceutical composition according to one or more embodiments of the disclosure may, if desired, contain a combination of pharmaceutical agent for treatment of pulmonary diseases (e.g., imatinib free base, imatinib salt) and one or more additional active agents. The pharmaceutical compositions can be aerosolized prior to administration or can be presented to a user in the form of an aerosol.

In some cases, all the starting materials are sterilized by established technologies that meet the standards for medical use. Typically, manufacturing equipment is sterilized before use. Some or all of other additional pharmaceutically acceptable carrier or excipient, solubilizer, or other additional ingredients of the pharmaceutical composition (e.g., cyclodextrin, e.g., SBEβCD or HPβCD; e.g., acids, e.g., acetic acid, hydrochloric acid, nitric acid, or citric acid; e.g., saccharin, e.g., saccharin sodium, e.g., lipid or fatty acid, e.g., co-solvent) can added into a suitable container.

In some cases, the kits include separate containers/receptacles for containing the pharmaceutical composition as described herein. In some other cases, the kits include a single container for containing the pharmaceutical composition. The kits can further include instructions for methods of using the kit. The instructions can be presented in the form of a data sheet, a manual, in a piece of paper, printed on one or more containers or devices of the kit. Alternatively, the instructions can be provided in electronic form, for instance, available in a disc or online with a weblink available from the kit. The instructions for use of the kit can comprise instructions for use of the pharmaceutical composition and the aerosolization device (e.g., a nebulizer) to treat any applicable indication, e.g., pulmonary disease. The instructions for use of the kit can comprise instructions for use of the pharmaceutical composition and the aerosolization device (e.g., a nebulizer) to treat a pulmonary disease. In some cases, the kits include a nose clip. A nose clip can be used to hinder passage of air through a nose of a subject during inhalation and increase the proportion of a total inhaled volume that is the aerosol issued by the a nebulizer.

Unit doses of the pharmaceutical compositions can be placed in a container. The container can be inserted into an aerosolization device. The container can be of a suitable shape, size, and material to contain the pharmaceutical composition and to provide the pharmaceutical composition in a usable condition. For example, the container can comprise a wall which comprises a material that does not adversely react with the pharmaceutical composition. In addition, the wall can comprise a material that allows the capsule to be opened to allow the pharmaceutical composition to be aerosolized.

In some cases, the pharmaceutical composition in the container is stable in reduced temperatures (e.g., 2-8° C.) for an extended period of time and may prolong the stability of the pharmaceutical composition.

### TERMINOLOGY

As used herein, the singular forms “a,” “an,” and “the” can include plural referents unless the context clearly dictates otherwise. Thus, for example, reference to “pharmaceutical agent for treatment of pulmonary diseases” can include not only a single active agent but also a combination or mixture of two or more different active agents.

Reference herein to “one embodiment,” “one version,” or “one aspect” can include one or more such embodiments, versions or aspects, unless otherwise clear from the context.

As used herein, the term “pharmaceutically acceptable solvate” can refer to a solvate that retains one or more of the biological activities and/or properties of the pharmaceutical agent and that is not biologically or otherwise undesirable. Examples of pharmaceutically acceptable solvates include, but are not limited to, pharmaceutical agents for treatment of pulmonary diseases in combination with water, isopropanol, ethanol, methanol, DMSO, ethyl acetate, acetic acid, ethanolamine, or combinations thereof.

As used herein, the term “salt” is equivalent to the term “pharmaceutically acceptable salt,” and can refer to those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfates, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, sulfonates, xylenesulfonates, phenylacetates, phenyipropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, the terms “treating” and “treatment” can refer to reduction in severity and/or frequency of symptoms, elimination of symptoms and/or underlying cause, reduction in likelihood of the occurrence of symptoms and/or underlying cause, and/or remediation of damage. Thus, “treating” a patient with an active agent as provided herein can include prevention of a particular condition, disease, or disorder in a susceptible individual as well as treatment of a clinically symptomatic individual.

As used herein, “nominal amount” can refer to the amount contained within the unit dose receptacle(s) that are administered.

As used herein, “effective amount” can refer to an amount covering both therapeutically effective amounts and prophylactically effective amounts.

As used herein, a “therapeutically effective amount” of an active agent can refer to an amount that is effective to achieve a desired therapeutic result. A therapeutically effective amount of a given active agent can vary with respect to factors such as the type and severity of the disorder or disease being treated and the age, gender, and weight of the patient. In some cases, “inhalation” (e.g., “oral inhalation” or “nasal inhalation”) refers to inhalation delivery of a therapeutically effective amount of a pharmaceutical agent contained in one unit dose receptacle, which, in some instance, can require one or more breaths, like 1, 2, 3, 4, 5, 6, 7, 8, 9, or more breaths. For example, if the effective amount is 90 mg, and each unit dose receptacle contains 30 mg, the delivery of the effective amount can require 3 inhalations.

Unless otherwise specified, the term “therapeutically effective amount” can include a “prophylactically effective amount,” e.g., an amount of active agent that is effective to prevent the onset or recurrence of a particular condition, disease, or disorder in a susceptible individual.

As used herein, the phrase “minimum effective amount” can mean the minimum amount of a pharmaceutical agent necessary to achieve an effective amount.

As used herein, “mass median diameter” or “MMD” can refer to the median diameter of a plurality of droplets, typically in a polydisperse droplet population, e.g., consisting of a range of droplet sizes.

As used herein, “particle” can refer to “droplet” of aerosolized pharmaceutical composition.

As used herein, “geometric diameter” can refer to the diameter of a single droplet, as determined by microscopy, unless the context indicates otherwise.

As used herein, “mass median aerodynamic diameter” or “MMAD” can refer to the median aerodynamic size of a plurality of droplets or droplets, typically in a polydisperse population. The “aerodynamic diameter” can be the diameter of a unit density sphere having the same settling velocity, generally in air, as a powder and is therefore a useful way to characterize an aerosolized powder or other dispersed droplet or droplet formulation in terms of its settling behavior. The aerodynamic diameter encompasses droplet or droplet shape, density, and physical size of the droplet or droplet. As used herein, MMAD refers to the median of the aerodynamic droplet or droplet size distribution of aerosolized droplets determined by cascade impaction, unless the context indicates otherwise.

By a “pharmaceutically acceptable” component is meant a component that is not biologically or otherwise undesirable, e.g., the component can be incorporated into a pharmaceutical composition of the disclosure and administered to a patient as described herein without causing any significant undesirable biological effects or interacting in a deleterious manner with any of the other components of the formulation in which it is contained. When the term “pharmaceutically acceptable” is used to refer to an excipient, it can imply that the component has met the required standards of toxicological and manufacturing testing or that it is included on the Inactive Ingredient Guide prepared by the U.S. Food and Drug Administration.

As used herein, “active nebulizer” or “nebulizer” refers to an inhalation device that does not rely solely on a patient's inspiratory effort to disperse and aerosolize a pharmaceutical composition contained within the device in a reservoir or in a unit dose form and does include inhaler devices that comprise a means for providing energy to disperse and aerosolize the drug composition, Such as pressurized gas and vibrating or rotating elements.

As used herein, “room temperature” can refer to a temperature that is from 18° C. to 25° C.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art can alternatively be used.

### Example 1: Organoleptic Testing of Formulations in Solution

This example illustrates organoleptic properties of certain exemplary formulations of imatinib in liquid solution.

In one experiment, exemplary formulations of imatinib according to some embodiments of the present disclosure were prepared as shown in Table 1 and tested by volunteer subjects at a minimum volume for their organoleptic properties.

To prepare the exemplary formulations, imatinib mesylate or imatinib freebase was dissolved directly in sterile water or sterile aqueous solution that contains the designated excipient (e.g., propylene glycol, sodium saccharin, sodium chloride, lactose monohydrate, phosphate, dextrose anhydrous, or hydroxypropyl-β-cyclodextrin). The pH of the resultant solutions were measured, and when needed, water was added to make up the final solution. Each of the solutions were filtered through a 0.22 μm filter and the osmolality of the final solution was measured and determined.

Four individuals were instructed to conduct deep lung inhalation of a minimal volume (<1.5 mL) of one or more exemplary formulations at an interval of 1 to 2 minutes. Each individual was asked to describe the taste of the inhaled solution and record their cough reflex, if any, and their sensational feeling of the solution in throat and mouth both during and after inhalation. Table 2 is a summary of the observations made by the individuals. It was found that among the tested formulations, most of the formulations that were made of imatinib mesylate induced cough reflex of the tested individuals, and were reported to be irritative. In some cases, the testers could not complete the inhalation because of the strong cough reflex or irritation. In contrast, exemplary formulation 13 that was made of imatinib freebase did not induce cough reflex or induced very little cough or irritational sensation in the throat or mouth.

### Example 2: Exemplary Imatinib Formulations

Table 3 is list of exemplary imatinib formulations with various solubility enhancers according to certain embodiments of the present disclosure.

TABLE 1
Exemplary imatinib formulations for organoleptic test

| | Formula- | | | | | | | | | | | | | |
|---|---|---|---|---|---|---|---|---|---|---|---|---|---|---|
| Component | tion 0 (F0) | F1 | F2 | F3 | F4 | F5 | F6 | F7 | F8 | F9 | F10 | F11 | F12 | F13 |
| Imatinib mesylate in | 5 | 40 | 40 | 40 | 40 | 40 | 40 | 40 | 20 | 40 | 40 | 40 | 40 | — |
| water (mg/mL) | | | | | | | | | | | | | | |
| imatinib freebase in | — | — | — | — | — | — | — | — | — | — | — | — | — | 7 |
| water (mg/mL) | | | | | | | | | | | | | | |
| Propylene glycol (mM) | — | — | 100 | — | 100 | 200 | 250 | 300 | 100 | — | — | — | — | — |
| Sodium Saccharin (mM) | — | — | — | 0.75 | 0.75 | 0.75 | 0.75 | 0.75 | 0.375 | — | — | 0.75 | — | — |
| Sodium Chloride (mM) | — | — | — | — | — | — | — | — | 77 | — | — | — | — | — |
| Lactose Monohydrate | — | — | — | — | — | — | — | — | — | 50 | 150 | 150 | 75 | — |
| (mg/mL) | | | | | | | | | | | | | | |
| Phosphate (mM) | — | — | — | — | — | — | — | — | — | 75 | — | — | — | — |
| Dextrose Anhydrous | — | — | — | — | — | — | — | — | — | — | — | — | 25 | — |
| (mg/mL) | | | | | | | | | | | | | | |
| Hydroxypropyl-b- | — | — | — | — | — | — | — | — | — | — | — | — | — | 40 |
| cyclodextrin (% w/v) | | | | | | | | | | | | | | |
| pH | 5.05 | 5.05 | 5.07 | 5.07 | 5.0 | 5 | 5 | 5 | 5 | 5 | 5 | 5 | 5 | 5 |
| Osmolality | 11 | 67.8 | 167.8 | 69.3 | 269 | 269 | 319 | 302 | 322.6 | 337 | 316 | 318 | 329 | 303 |

TABLE 2
Summary of organoleptic testing of exemplary formulations

| Formulation | | F0 | F1 | F4 | F4 | F6 | F7 |
|---|---|---|---|---|---|---|---|
| Tester | | Tester 1 | T1 | T1 | T3 | T1 | T1 |
| | | (T1) | | | | | |
| Inhalation | Taste | No | No | No | No | No | No |
| | Cough reflex | Yes | Strong urge | Strong urge to | Yes | Strong urge | No |
| | | | to cough | cough after two | | to cough | |
| | | | immediately | inhalations | | after about | |
| | | | during first | | | a minute of | |
| | | | deep | | | inhalation | |
| | | | inhalation | | | (four or | |
| | | | | | | five deep | |
| | | | | | | breaths) | |
| | Cough? | Yes - 35 s into | Yes, | Yes, | Intense and | Yes, | No |
| | | inhalation, not | persistent, | persistent, | sustained | persistent, | |
| | | severe | strong | strong | | strong | |
| | Sensation in | No | No | No | no irritation | No | No |
| | the throat | | | | no burning | | |
| | (Irritation, | | | | no numbness | | |
| | Tingling, | | | | Tingling? | | |
| | Numbness, | | | | | | |
| | Burning) | | | | | | |
| | Mouthfeel | No | No | No | n/a | No | No |
| | (smooth? | | | | | | |
| | Astringent?) | | | | | | |
| | Other? | | | | | | |
| Post- | Taste | none | No | No | none | No | No |
| inhalation | | | | | | | |
| (for any of the | | | | | | | |
| observations | | | | | | | |
| above) | | | | | | | |
| | Cough reflex | Slight | Persisted for | Persisted for | | Persisted for | No |
| | | | about 30 | about 5 to | | about 5 | |
| | | | minutes | 10 minutes, | | minutes, | |
| | | | | ameliorated | | stopped upon | |
| | | | | by inhaling | | inhaling | |
| | | | | saline | | saline | |
| | Cough? | None | Persisted for | Persisted for | cough | Persisted for | No |
| | | | about 30 | about 5 to | continued | about 5 | |
| | | | minutes | 10 minutes, | for ~5 min | minutes, | |
| | | | | ameliorated | | stopped upon | |
| | | | | by inhaling | | inhaling | |
| | | | | saline | | saline | |
| | Sensation in the | | Persisted for | Persisted for | none | Persisted for | No |
| | throat | | about 30 | about 5 to | | about 5 | |
| | (Irritation, | | minutes | 10 minutes, | | minutes, | |
| | Tingling, | | | ameliorated | | stopped upon | |
| | Numbness, or | | | by inhaling | | inhaling | |
| | Burning) | | | saline | | saline | |
| | Mouthfeel | | No | No | none | No | No |
| | (Astringent?) | | | | | | |
| | Other feeling? | | | | | | |
| How long | | NA | About 30 | Persisted for | ~10 min | Persisted for | NA |
| does it take | | | minutes | about 5 to 10 | | about 5 | |
| for the throat | | | | minutes, | | minutes, | |
| to feel | | | | ameliorated | | stopped upon | |
| normal | | | | by inhaling | | inhaling | |
| again? | | | | saline | | saline | |

| Formulation | | F8 | F8 | F9 | F12 | F13 | F13 |
|---|---|---|---|---|---|---|---|
| Tester | | T1 | T2 | T4 | T1 | T1 | T4 |
| Inhalation | Taste | No | Smell of vapor | No | Somewhat | No | No |
| | | | like “new | | Bitter | | |
| | | | plastic” | | | | |
| | Cough reflex | Strong urge | Yes, From lungs, | Yes after | After 1 | No | Slightly |
| | | to cough | not from throat. | 5-10 | minute, | | tickling in |
| | | after about a | | breaths | induced a | | throat |
| | | minute and | | | persistent | | after 7 |
| | | a half of | | | cough and | | breaths, |
| | | inhalation | | | strong mucous | | coughed |
| | | (More than | | | reaction | | and was |
| | | six breaths) | | | | | fine |
| | Cough? | Yes, | Yes | Yes, | Yes, | No | 1 or 2 |
| | | persistent, | | lasting 5- | persistent, | | |
| | | strong | | 10 secs | strong | | |
| | Sensation in | No | Can feel vapors | No | No | No | No |
| | the throat | | going down but, | | | | |
| | (Irritation, | | no throat | | | | |
| | Tingling, | | irritation during. | | | | |
| | Numbness, | | | | | | |
| | Burning) | | | | | | |
| | Mouthfeel | No | Mostly vapory | No | No | No | No |
| | (smooth? | | and slightly | | | | |
| | Astringent?) | | sweet after | | | | |
| | Other? | | Uncontrollable | | | | |
| | | | cough reflex | | | | |
| | | | after 5 deep | | | | |
| | | | inhalations (~30 | | | | |
| | | | sec); Came on | | | | |
| | | | almost | | | | |
| | | | instanteously, did | | | | |
| | | | not feel it | | | | |
| | | | building, but | | | | |
| | | | once set off, it | | | | |
| | | | could not be | | | | |
| | | | suppressed or | | | | |
| | | | controlled. | | | | |
| Post- | Taste | No | Little sweetness | No | No | No | No |
| inhalation | | | | | | | |
| (for any of the | | | | | | | |
| observations | | | | | | | |
| above) | | | | | | | |
| | Cough reflex | Persisted for | Continued deep | yes | Persisted, | No | No |
| | | about 5 | lung cough for 5 | | stopped upon | reaction | |
| | | minutes, | minutes, tapering | | inhaling saline | after 2 | |
| | | stopped upon | down. Still feel | | | mins | |
| | | inhaling | urge to cough up | | | | |
| | | saline | to 20 min after. | | | | |
| | Cough? | Persisted for | Yes, diminishing | mild | Persisted, | No | No |
| | | about 5 | after 5 min, but | | stopped upon | reaction | |
| | | minutes, | still residual up | | inhaling saline | after 2 | |
| | | stopped upon | to 20 min post | | | mins | |
| | | inhaling | completion. | | | | |
| | | saline | | | | | |
| | Sensation in the | Persisted for | None | No | Persisted, | No | No |
| | throat | about 5 | | | stopped upon | reaction | |
| | (Irritation, | minutes, | | | inhaling saline | after 2 | |
| | Tingling, | stopped upon | | | | mins | |
| | Numbness, or | inhaling | | | | | |
| | Burning) | saline | | | | | |
| | Mouthfeel | No | None | No | No | No | No |
| | (Astringent?) | | | | | | |
| | Other feeling? | | | | | | |
| How long | | Persisted for | NA | NA | NA | NA | NA |
| does it take | | about 5 | | | | | |
| for the throat | | minutes, | | | | | |
| to feel | | stopped upon | | | | | |
| normal | | inhaling | | | | | |
| again? | | saline | | | | | |

TABLE 3
Exemplary imatinib formulations in aqueous solution

| | Imatinib | | | | | | | PEG | PEG | | | | |
|---|---|---|---|---|---|---|---|---|---|---|---|---|---|
| | freebase | α-CD | β-CD | γ-CD | HPβCD | MβCD | SBEβCD | 300 | 400 | PG | Ethanol | | pH |
| Formulations | (mg/mL) | (% w/v) | (% w/v) | (% w/v) | (% w/v) | (% w/v) | (% w/v) | (% v/v) | (% v/v) | (% v/v) | (% v/v) | pH | buffer |
| 14 | 20 | 5 | | | | | | | | | | 5 | phosphate |
| 15 | 20 | 10 | | | | | | | | | | 5 | phosphate |
| 16 | 20 | 20 | | | | | | | | | | 5 | phosphate |
| 17 | 20 | 30 | | | | | | | | | | 5 | phosphate |
| 18 | 20 | | 5 | | | | | | | | | 5 | phosphate |
| 19 | 20 | | 10 | | | | | | | | | 5 | phosphate |
| 20 | 20 | | 20 | | | | | | | | | 5 | phosphate |
| 21 | 20 | | 30 | | | | | | | | | 5 | phosphate |
| 22 | 20 | | | 5 | | | | | | | | 5 | phosphate |
| 23 | 20 | | | 10 | | | | | | | | 5 | phosphate |
| 24 | 20 | | | 20 | | | | | | | | 5 | phosphate |
| 25 | 20 | | | 30 | | | | | | | | 5 | phosphate |
| 26 | 20 | | | | 5 | | | | | | | 5 | phosphate |
| 27 | 20 | | | | 10 | | | | | | | 5 | phosphate |
| 28 | 20 | | | | 20 | | | | | | | 5 | phosphate |
| 29 | 20 | | | | 30 | | | | | | | 5 | phosphate |
| 30 | 20 | | | | | 5 | | | | | | 5 | phosphate |
| 31 | 20 | | | | | 10 | | | | | | 5 | phosphate |
| 32 | 20 | | | | | 20 | | | | | | 5 | phosphate |
| 33 | 20 | | | | | 30 | | | | | | 5 | phosphate |
| 34 | 20 | | | | | | 5 | | | | | 5 | phosphate |
| 35 | 20 | | | | | | 10 | | | | | 5 | phosphate |
| 36 | 20 | | | | | | 20 | | | | | 5 | phosphate |
| 37 | 20 | | | | | | 30 | | | | | 5 | phosphate |
| 38 | 20 | | | | | | | 10 | | | | 5 | phosphate |
| 39 | 20 | | | | | | | 20 | | | | 5 | phosphate |
| 40 | 20 | | | | | | | 100 | | | | 5 | phosphate |
| 41 | 20 | | | | | | | | 10 | | | 5 | phosphate |
| 42 | 20 | | | | | | | | 20 | | | 5 | phosphate |
| 43 | 20 | | | | | | | | 100 | | | 5 | phosphate |
| 44 | 20 | | | | | | | | | 10 | | 5 | phosphate |
| 45 | 20 | | | | | | | | | 20 | | 5 | phosphate |
| 46 | 20 | | | | | | | | | 100 | | 5 | phosphate |
| 47 | 20 | | | | | | | | | | 5 | 5 | phosphate |
| 48 | 20 | | | | | | | | | | 10 | 5 | phosphate |
| 49 | 20 | | | | | | | | | | 20 | 5 | phosphate |
| 50 | 20 | | | | | | | | | | 100 | 5 | phosphate |
| 51 | 30 | 5 | | | | | | | | | | 5 | phosphate |
| 52 | 30 | 10 | | | | | | | | | | 5 | phosphate |
| 53 | 30 | 20 | | | | | | | | | | 5 | phosphate |
| 54 | 30 | 30 | | | | | | | | | | 5 | phosphate |
| 55 | 30 | | 5 | | | | | | | | | 5 | phosphate |
| 56 | 30 | | 10 | | | | | | | | | 5 | phosphate |
| 57 | 30 | | 20 | | | | | | | | | 5 | phosphate |
| 58 | 30 | | 30 | | | | | | | | | 5 | phosphate |
| 59 | 30 | | | 5 | | | | | | | | 5 | phosphate |
| 60 | 30 | | | 10 | | | | | | | | 5 | phosphate |
| 61 | 30 | | | 20 | | | | | | | | 5 | phosphate |
| 62 | 30 | | | 30 | | | | | | | | 5 | phosphate |
| 63 | 30 | | | | 5 | | | | | | | 5 | phosphate |
| 64 | 30 | | | | 10 | | | | | | | 5 | phosphate |
| 65 | 30 | | | | 20 | | | | | | | 5 | phosphate |
| 66 | 30 | | | | 30 | | | | | | | 5 | phosphate |
| 67 | 30 | | | | | 5 | | | | | | 5 | phosphate |
| 68 | 30 | | | | | 10 | | | | | | 5 | phosphate |
| 69 | 30 | | | | | 20 | | | | | | 5 | phosphate |
| 70 | 30 | | | | | 30 | | | | | | 5 | phosphate |
| 71 | 30 | | | | | | 5 | | | | | 5 | phosphate |
| 72 | 30 | | | | | | 10 | | | | | 5 | phosphate |
| 73 | 30 | | | | | | 20 | | | | | 5 | phosphate |
| 74 | 30 | | | | | | 30 | | | | | 5 | phosphate |
| 75 | 30 | | | | | | | 10 | | | | 5 | phosphate |
| 76 | 30 | | | | | | | 20 | | | | 5 | phosphate |
| 77 | 30 | | | | | | | 100 | | | | 5 | phosphate |
| 78 | 30 | | | | | | | | 10 | | | 5 | phosphate |
| 79 | 30 | | | | | | | | 20 | | | 5 | phosphate |
| 80 | 30 | | | | | | | | 100 | | | 5 | phosphate |
| 81 | 30 | | | | | | | | | 10 | | 5 | phosphate |
| 82 | 30 | | | | | | | | | 20 | | 5 | phosphate |
| 83 | 30 | | | | | | | | | 100 | | 5 | phosphate |
| 84 | 30 | | | | | | | | | | 5 | 5 | phosphate |
| 85 | 30 | | | | | | | | | | 10 | 5 | phosphate |
| 86 | 30 | | | | | | | | | | 20 | 5 | phosphate |
| 87 | 30 | | | | | | | | | | 100 | 5 | phosphate |
| 88 | 40 | 5 | | | | | | | | | | 5 | phosphate |
| 89 | 40 | 10 | | | | | | | | | | 5 | phosphate |
| 90 | 40 | 20 | | | | | | | | | | 5 | phosphate |
| 91 | 40 | 30 | | | | | | | | | | 5 | phosphate |
| 92 | 40 | | 5 | | | | | | | | | 5 | phosphate |
| 93 | 40 | | 10 | | | | | | | | | 5 | phosphate |
| 94 | 40 | | 20 | | | | | | | | | 5 | phosphate |
| 95 | 40 | | 30 | | | | | | | | | 5 | phosphate |
| 96 | 40 | | | 5 | | | | | | | | 5 | phosphate |
| 97 | 40 | | | 10 | | | | | | | | 5 | phosphate |
| 98 | 40 | | | 20 | | | | | | | | 5 | phosphate |
| 99 | 40 | | | 30 | | | | | | | | 5 | phosphate |
| 100 | 40 | | | | 5 | | | | | | | 5 | phosphate |
| 101 | 40 | | | | 10 | | | | | | | 5 | phosphate |
| 102 | 40 | | | | 20 | | | | | | | 5 | phosphate |
| 103 | 40 | | | | 30 | | | | | | | 5 | phosphate |
| 104 | 40 | | | | | 5 | | | | | | 5 | phosphate |
| 105 | 40 | | | | | 10 | | | | | | 5 | phosphate |
| 106 | 40 | | | | | 20 | | | | | | 5 | phosphate |
| 107 | 40 | | | | | 30 | | | | | | 5 | phosphate |
| 108 | 40 | | | | | | 5 | | | | | 5 | phosphate |
| 109 | 40 | | | | | | 10 | | | | | 5 | phosphate |
| 110 | 40 | | | | | | 20 | | | | | 5 | phosphate |
| 111 | 40 | | | | | | 30 | | | | | 5 | phosphate |
| 112 | 40 | | | | | | | 10 | | | | 5 | phosphate |
| 113 | 40 | | | | | | | 20 | | | | 5 | phosphate |
| 114 | 40 | | | | | | | 100 | | | | 5 | phosphate |
| 115 | 40 | | | | | | | | 10 | | | 5 | phosphate |
| 116 | 40 | | | | | | | | 20 | | | 5 | phosphate |
| 117 | 40 | | | | | | | | 100 | | | 5 | phosphate |
| 118 | 40 | | | | | | | | | 10 | | 5 | phosphate |
| 119 | 40 | | | | | | | | | 20 | | 5 | phosphate |
| 120 | 40 | | | | | | | | | 100 | | 5 | phosphate |
| 121 | 40 | | | | | | | | | | 5 | 5 | phosphate |
| 122 | 40 | | | | | | | | | | 10 | 5 | phosphate |
| 123 | 40 | | | | | | | | | | 20 | 5 | phosphate |
| 124 | 40 | | | | | | | | | | 100 | 5 | phosphate |
| 125 | 50 | | | | | | 10 | | | | | 5 | phosphate |
| 126 | 50 | | | | | | 20 | | | | | 5 | phosphate |
| 127 | 50 | | | | | | 30 | | | | | 5 | phosphate |
| 128 | 50 | | | | | | 40 | | | | | 5 | phosphate |
| 129 | 50 | | | | | | 50 | | | | | 5 | phosphate |
| 130 | 100 | | | | | | 20 | | | | | 5 | phosphate |
| 131 | 100 | | | | | | 30 | | | | | 5 | phosphate |
| 132 | 100 | | | | | | 40 | | | | | 5 | phosphate |
| 133 | 100 | | | | | | 50 | | | | | 5 | phosphate |
| 134 | 150 | | | | | | 20 | | | | | 5 | phosphate |
| 135 | 150 | | | | | | 30 | | | | | 5 | phosphate |
| 136 | 150 | | | | | | 40 | | | | | 5 | phosphate |
| 137 | 150 | | | | | | 50 | | | | | 5 | phosphate |
| 138 | 200 | | | | | | 30 | | | | | 5 | phosphate |
| 139 | 200 | | | | | | 40 | | | | | 5 | phosphate |
| 140 | 200 | | | | | | 50 | | | | | 5 | phosphate |
| 141 | 250 | | | | | | 30 | | | | | 5 | phosphate |
| 142 | 250 | | | | | | 40 | | | | | 5 | phosphate |
| 143 | 250 | | | | | | 50 | | | | | 5 | phosphate |
| 144 | 300 | | | | | | 30 | | | | | 5 | phosphate |
| 145 | 300 | | | | | | 40 | | | | | 5 | phosphate |
| 146 | 300 | | | | | | 50 | | | | | 5 | phosphate |
| 147 | 350 | | | | | | 40 | | | | | 5 | phosphate |
| 148 | 400 | | | | | | 50 | | | | | 5 | phosphate |
| 149 | 400 | | | | | | 40 | | | | | 5 | phosphate |
| 150 | 450 | | | | | | 50 | | | | | 5 | phosphate |
| 151 | 500 | | | | | | 50 | | | | | 5 | phosphate |
| 152 | 20 | 5 | | | | | | | | | | 6 | phosphate |
| 153 | 20 | 10 | | | | | | | | | | 6 | phosphate |
| 154 | 20 | 20 | | | | | | | | | | 6 | phosphate |
| 155 | 20 | 30 | | | | | | | | | | 6 | phosphate |
| 156 | 20 | | 5 | | | | | | | | | 6 | phosphate |
| 157 | 20 | | 10 | | | | | | | | | 6 | phosphate |
| 158 | 20 | | 20 | | | | | | | | | 6 | phosphate |
| 159 | 20 | | 30 | | | | | | | | | 6 | phosphate |
| 160 | 20 | | | 5 | | | | | | | | 6 | phosphate |
| 161 | 20 | | | 10 | | | | | | | | 6 | phosphate |
| 162 | 20 | | | 20 | | | | | | | | 6 | phosphate |
| 163 | 20 | | | 30 | | | | | | | | 6 | phosphate |
| 164 | 20 | | | | 5 | | | | | | | 6 | phosphate |
| 165 | 20 | | | | 10 | | | | | | | 6 | phosphate |
| 166 | 20 | | | | 20 | | | | | | | 6 | phosphate |
| 167 | 20 | | | | 30 | | | | | | | 6 | phosphate |
| 168 | 20 | | | | | 5 | | | | | | 6 | phosphate |
| 169 | 20 | | | | | 10 | | | | | | 6 | phosphate |
| 170 | 20 | | | | | 20 | | | | | | 6 | phosphate |
| 171 | 20 | | | | | 30 | | | | | | 6 | phosphate |
| 172 | 20 | | | | | | 5 | | | | | 6 | phosphate |
| 173 | 20 | | | | | | 10 | | | | | 6 | phosphate |
| 174 | 20 | | | | | | 20 | | | | | 6 | phosphate |
| 175 | 20 | | | | | | 30 | | | | | 6 | phosphate |
| 176 | 20 | | | | | | | 10 | | | | 6 | phosphate |
| 177 | 20 | | | | | | | 20 | | | | 6 | phosphate |
| 178 | 20 | | | | | | | 100 | | | | 6 | phosphate |
| 179 | 20 | | | | | | | | 10 | | | 6 | phosphate |
| 180 | 20 | | | | | | | | 20 | | | 6 | phosphate |
| 181 | 20 | | | | | | | | 100 | | | 6 | phosphate |
| 182 | 20 | | | | | | | | | 10 | | 6 | phosphate |
| 183 | 20 | | | | | | | | | 20 | | 6 | phosphate |
| 184 | 20 | | | | | | | | | 100 | | 6 | phosphate |
| 185 | 20 | | | | | | | | | | 5 | 6 | phosphate |
| 186 | 20 | | | | | | | | | | 10 | 6 | phosphate |
| 187 | 20 | | | | | | | | | | 20 | 6 | phosphate |
| 188 | 20 | | | | | | | | | | 100 | 6 | phosphate |
| 189 | 30 | 5 | | | | | | | | | | 6 | phosphate |
| 190 | 30 | 10 | | | | | | | | | | 6 | phosphate |
| 191 | 30 | 20 | | | | | | | | | | 6 | phosphate |
| 192 | 30 | 30 | | | | | | | | | | 6 | phosphate |
| 193 | 30 | | 5 | | | | | | | | | 6 | phosphate |
| 194 | 30 | | 10 | | | | | | | | | 6 | phosphate |
| 195 | 30 | | 20 | | | | | | | | | 6 | phosphate |
| 196 | 30 | | 30 | | | | | | | | | 6 | phosphate |
| 197 | 30 | | | 5 | | | | | | | | 6 | phosphate |
| 198 | 30 | | | 10 | | | | | | | | 6 | phosphate |
| 199 | 30 | | | 20 | | | | | | | | 6 | phosphate |
| 200 | 30 | | | 30 | | | | | | | | 6 | phosphate |
| 201 | 30 | | | | 5 | | | | | | | 6 | phosphate |
| 202 | 30 | | | | 10 | | | | | | | 6 | phosphate |
| 203 | 30 | | | | 20 | | | | | | | 6 | phosphate |
| 204 | 30 | | | | 30 | | | | | | | 6 | phosphate |
| 205 | 30 | | | | | 5 | | | | | | 6 | phosphate |
| 206 | 30 | | | | | 10 | | | | | | 6 | phosphate |
| 207 | 30 | | | | | 20 | | | | | | 6 | phosphate |
| 208 | 30 | | | | | 30 | | | | | | 6 | phosphate |
| 209 | 30 | | | | | | 5 | | | | | 6 | phosphate |
| 210 | 30 | | | | | | 10 | | | | | 6 | phosphate |
| 211 | 30 | | | | | | 20 | | | | | 6 | phosphate |
| 212 | 30 | | | | | | 30 | | | | | 6 | phosphate |
| 213 | 30 | | | | | | | 10 | | | | 6 | phosphate |
| 214 | 30 | | | | | | | 20 | | | | 6 | phosphate |
| 215 | 30 | | | | | | | 100 | | | | 6 | phosphate |
| 216 | 30 | | | | | | | | 10 | | | 6 | phosphate |
| 217 | 30 | | | | | | | | 20 | | | 6 | phosphate |
| 218 | 30 | | | | | | | | 100 | | | 6 | phosphate |
| 219 | 30 | | | | | | | | | 10 | | 6 | phosphate |
| 220 | 30 | | | | | | | | | 20 | | 6 | phosphate |
| 221 | 30 | | | | | | | | | 100 | | 6 | phosphate |
| 222 | 30 | | | | | | | | | | 5 | 6 | phosphate |
| 223 | 30 | | | | | | | | | | 10 | 6 | phosphate |
| 224 | 30 | | | | | | | | | | 20 | 6 | phosphate |
| 225 | 30 | | | | | | | | | | 100 | 6 | phosphate |
| 226 | 40 | 5 | | | | | | | | | | 6 | phosphate |
| 227 | 40 | 10 | | | | | | | | | | 6 | phosphate |
| 228 | 40 | 20 | | | | | | | | | | 6 | phosphate |
| 229 | 40 | 30 | | | | | | | | | | 6 | phosphate |
| 230 | 40 | | 5 | | | | | | | | | 6 | phosphate |
| 231 | 40 | | 10 | | | | | | | | | 6 | phosphate |
| 232 | 40 | | 20 | | | | | | | | | 6 | phosphate |
| 233 | 40 | | 30 | | | | | | | | | 6 | phosphate |
| 234 | 40 | | | 5 | | | | | | | | 6 | phosphate |
| 235 | 40 | | | 10 | | | | | | | | 6 | phosphate |
| 236 | 40 | | | 20 | | | | | | | | 6 | phosphate |
| 237 | 40 | | | 30 | | | | | | | | 6 | phosphate |
| 238 | 40 | | | | 5 | | | | | | | 6 | phosphate |
| 239 | 40 | | | | 10 | | | | | | | 6 | phosphate |
| 240 | 40 | | | | 20 | | | | | | | 6 | phosphate |
| 241 | 40 | | | | 30 | | | | | | | 6 | phosphate |
| 242 | 40 | | | | | 5 | | | | | | 6 | phosphate |
| 243 | 40 | | | | | 10 | | | | | | 6 | phosphate |
| 244 | 40 | | | | | 20 | | | | | | 6 | phosphate |
| 245 | 40 | | | | | 30 | | | | | | 6 | phosphate |
| 246 | 40 | | | | | | 5 | | | | | 6 | phosphate |
| 247 | 40 | | | | | | 10 | | | | | 6 | phosphate |
| 248 | 40 | | | | | | 20 | | | | | 6 | phosphate |
| 249 | 40 | | | | | | 30 | | | | | 6 | phosphate |
| 250 | 40 | | | | | | | 10 | | | | 6 | phosphate |
| 251 | 40 | | | | | | | 20 | | | | 6 | phosphate |
| 252 | 40 | | | | | | | 100 | | | | 6 | phosphate |
| 253 | 40 | | | | | | | | 10 | | | 6 | phosphate |
| 254 | 40 | | | | | | | | 20 | | | 6 | phosphate |
| 255 | 40 | | | | | | | | 100 | | | 6 | phosphate |
| 256 | 40 | | | | | | | | | 10 | | 6 | phosphate |
| 257 | 40 | | | | | | | | | 20 | | 6 | phosphate |
| 258 | 40 | | | | | | | | | 100 | | 6 | phosphate |
| 259 | 40 | | | | | | | | | | 5 | 6 | phosphate |
| 260 | 40 | | | | | | | | | | 10 | 6 | phosphate |
| 261 | 40 | | | | | | | | | | 20 | 6 | phosphate |
| 262 | 40 | | | | | | | | | | 100 | 6 | phosphate |
| 263 | 50 | | | | | | 10 | | | | | 6 | phosphate |
| 264 | 50 | | | | | | 20 | | | | | 6 | phosphate |
| 265 | 50 | | | | | | 30 | | | | | 6 | phosphate |
| 266 | 50 | | | | | | 40 | | | | | 6 | phosphate |
| 267 | 50 | | | | | | 50 | | | | | 6 | phosphate |
| 268 | 100 | | | | | | 20 | | | | | 6 | phosphate |
| 269 | 100 | | | | | | 30 | | | | | 6 | phosphate |
| 270 | 100 | | | | | | 40 | | | | | 6 | phosphate |
| 271 | 100 | | | | | | 50 | | | | | 6 | phosphate |
| 272 | 150 | | | | | | 20 | | | | | 6 | phosphate |
| 273 | 150 | | | | | | 30 | | | | | 6 | phosphate |
| 274 | 150 | | | | | | 40 | | | | | 6 | phosphate |
| 275 | 150 | | | | | | 50 | | | | | 6 | phosphate |
| 276 | 200 | | | | | | 30 | | | | | 6 | phosphate |
| 277 | 200 | | | | | | 40 | | | | | 6 | phosphate |
| 278 | 200 | | | | | | 50 | | | | | 6 | phosphate |
| 279 | 250 | | | | | | 30 | | | | | 6 | phosphate |
| 280 | 250 | | | | | | 40 | | | | | 6 | phosphate |
| 281 | 250 | | | | | | 50 | | | | | 6 | phosphate |
| 282 | 300 | | | | | | 30 | | | | | 6 | phosphate |
| 283 | 300 | | | | | | 40 | | | | | 6 | phosphate |
| 284 | 300 | | | | | | 50 | | | | | 6 | phosphate |
| 285 | 350 | | | | | | 40 | | | | | 6 | phosphate |
| 286 | 400 | | | | | | 50 | | | | | 6 | phosphate |
| 287 | 400 | | | | | | 40 | | | | | 6 | phosphate |
| 288 | 450 | | | | | | 50 | | | | | 6 | phosphate |
| 289 | 500 | | | | | | 50 | | | | | 6 | phosphate |
| 290 | 20 | 5 | | | | | | | | | | 5 | citrate |
| 291 | 20 | 10 | | | | | | | | | | 5 | citrate |
| 292 | 20 | 20 | | | | | | | | | | 5 | citrate |
| 293 | 20 | 30 | | | | | | | | | | 5 | citrate |
| 294 | 20 | | 5 | | | | | | | | | 5 | citrate |
| 295 | 20 | | 10 | | | | | | | | | 5 | citrate |
| 296 | 20 | | 20 | | | | | | | | | 5 | citrate |
| 297 | 20 | | 30 | | | | | | | | | 5 | citrate |
| 298 | 20 | | | 5 | | | | | | | | 5 | citrate |
| 299 | 20 | | | 10 | | | | | | | | 5 | citrate |
| 300 | 20 | | | 20 | | | | | | | | 5 | citrate |
| 301 | 20 | | | 30 | | | | | | | | 5 | citrate |
| 302 | 20 | | | | 5 | | | | | | | 5 | citrate |
| 303 | 20 | | | | 10 | | | | | | | 5 | citrate |
| 304 | 20 | | | | 20 | | | | | | | 5 | citrate |
| 305 | 20 | | | | 30 | | | | | | | 5 | citrate |
| 306 | 20 | | | | | 5 | | | | | | 5 | citrate |
| 307 | 20 | | | | | 10 | | | | | | 5 | citrate |
| 308 | 20 | | | | | 20 | | | | | | 5 | citrate |
| 309 | 20 | | | | | 30 | | | | | | 5 | citrate |
| 310 | 20 | | | | | | 5 | | | | | 5 | citrate |
| 311 | 20 | | | | | | 10 | | | | | 5 | citrate |
| 312 | 20 | | | | | | 20 | | | | | 5 | citrate |
| 313 | 20 | | | | | | 30 | | | | | 5 | citrate |
| 314 | 20 | | | | | | | 10 | | | | 5 | citrate |
| 315 | 20 | | | | | | | 20 | | | | 5 | citrate |
| 316 | 20 | | | | | | | 100 | | | | 5 | citrate |
| 317 | 20 | | | | | | | | 10 | | | 5 | citrate |
| 318 | 20 | | | | | | | | 20 | | | 5 | citrate |
| 319 | 20 | | | | | | | | 100 | | | 5 | citrate |
| 320 | 20 | | | | | | | | | 10 | | 5 | citrate |
| 321 | 20 | | | | | | | | | 20 | | 5 | citrate |
| 322 | 20 | | | | | | | | | 100 | | 5 | citrate |
| 323 | 20 | | | | | | | | | | 5 | 5 | citrate |
| 324 | 20 | | | | | | | | | | 10 | 5 | citrate |
| 325 | 20 | | | | | | | | | | 20 | 5 | citrate |
| 326 | 20 | | | | | | | | | | 100 | 5 | citrate |
| 327 | 30 | 5 | | | | | | | | | | 5 | citrate |
| 328 | 30 | 10 | | | | | | | | | | 5 | citrate |
| 329 | 30 | 20 | | | | | | | | | | 5 | citrate |
| 330 | 30 | 30 | | | | | | | | | | 5 | citrate |
| 331 | 30 | | 5 | | | | | | | | | 5 | citrate |
| 332 | 30 | | 10 | | | | | | | | | 5 | citrate |
| 333 | 30 | | 20 | | | | | | | | | 5 | citrate |
| 334 | 30 | | 30 | | | | | | | | | 5 | citrate |
| 335 | 30 | | | 5 | | | | | | | | 5 | citrate |
| 336 | 30 | | | 10 | | | | | | | | 5 | citrate |
| 337 | 30 | | | 20 | | | | | | | | 5 | citrate |
| 338 | 30 | | | 30 | | | | | | | | 5 | citrate |
| 339 | 30 | | | | 5 | | | | | | | 5 | citrate |
| 340 | 30 | | | | 10 | | | | | | | 5 | citrate |
| 341 | 30 | | | | 20 | | | | | | | 5 | citrate |
| 342 | 30 | | | | 30 | | | | | | | 5 | citrate |
| 343 | 30 | | | | | 5 | | | | | | 5 | citrate |
| 344 | 30 | | | | | 10 | | | | | | 5 | citrate |
| 345 | 30 | | | | | 20 | | | | | | 5 | citrate |
| 346 | 30 | | | | | 30 | | | | | | 5 | citrate |
| 347 | 30 | | | | | | 5 | | | | | 5 | citrate |
| 348 | 30 | | | | | | 10 | | | | | 5 | citrate |
| 349 | 30 | | | | | | 20 | | | | | 5 | citrate |
| 350 | 30 | | | | | | 30 | | | | | 5 | citrate |
| 351 | 30 | | | | | | | 10 | | | | 5 | citrate |
| 352 | 30 | | | | | | | 20 | | | | 5 | citrate |
| 353 | 30 | | | | | | | 100 | | | | 5 | citrate |
| 354 | 30 | | | | | | | | 10 | | | 5 | citrate |
| 355 | 30 | | | | | | | | 20 | | | 5 | citrate |
| 356 | 30 | | | | | | | | 100 | | | 5 | citrate |
| 357 | 30 | | | | | | | | | 10 | | 5 | citrate |
| 358 | 30 | | | | | | | | | 20 | | 5 | citrate |
| 359 | 30 | | | | | | | | | 100 | | 5 | citrate |
| 360 | 30 | | | | | | | | | | | 5 | citrate |
| 361 | 30 | | | | | | | | | | | 5 | citrate |
| 362 | 30 | | | | | | | | | | 5 | 5 | citrate |
| 363 | 30 | | | | | | | | | | 10 | 5 | citrate |
| 364 | 30 | | | | | | | | | | 20 | 5 | citrate |
| 365 | 30 | | | | | | | | | | 100 | 5 | citrate |
| 366 | 40 | 5 | | | | | | | | | | 5 | citrate |
| 367 | 40 | 10 | | | | | | | | | | 5 | citrate |
| 368 | 40 | 20 | | | | | | | | | | 5 | citrate |
| 369 | 40 | 30 | | | | | | | | | | 5 | citrate |
| 370 | 40 | | 5 | | | | | | | | | 5 | citrate |
| 371 | 40 | | 10 | | | | | | | | | 5 | citrate |
| 372 | 40 | | 20 | | | | | | | | | 5 | citrate |
| 373 | 40 | | 30 | | | | | | | | | 5 | citrate |
| 374 | 40 | | | 5 | | | | | | | | 5 | citrate |
| 375 | 40 | | | 10 | | | | | | | | 5 | citrate |
| 376 | 40 | | | 20 | | | | | | | | 5 | citrate |
| 377 | 40 | | | 30 | | | | | | | | 5 | citrate |
| 378 | 40 | | | | 5 | | | | | | | 5 | citrate |
| 379 | 40 | | | | 10 | | | | | | | 5 | citrate |
| 380 | 40 | | | | 20 | | | | | | | 5 | citrate |
| 381 | 40 | | | | 30 | | | | | | | 5 | citrate |
| 382 | 40 | | | | | 5 | | | | | | 5 | citrate |
| 383 | 40 | | | | | 10 | | | | | | 5 | citrate |
| 384 | 40 | | | | | 20 | | | | | | 5 | citrate |
| 385 | 40 | | | | | 30 | | | | | | 5 | citrate |
| 386 | 40 | | | | | | 5 | | | | | 5 | citrate |
| 387 | 40 | | | | | | 10 | | | | | 5 | citrate |
| 388 | 40 | | | | | | 20 | | | | | 5 | citrate |
| 389 | 40 | | | | | | 30 | | | | | 5 | citrate |
| 390 | 40 | | | | | | | 10 | | | | 5 | citrate |
| 391 | 40 | | | | | | | 20 | | | | 5 | citrate |
| 392 | 40 | | | | | | | 100 | | | | 5 | citrate |
| 393 | 40 | | | | | | | | 10 | | | 5 | citrate |
| 394 | 40 | | | | | | | | 20 | | | 5 | citrate |
| 395 | 40 | | | | | | | | 100 | | | 5 | citrate |
| 396 | 40 | | | | | | | | | 10 | | 5 | citrate |
| 397 | 40 | | | | | | | | | 20 | | 5 | citrate |
| 398 | 40 | | | | | | | | | 100 | | 5 | citrate |
| 399 | 40 | | | | | | | | | | 5 | 5 | citrate |
| 400 | 40 | | | | | | | | | | 10 | 5 | citrate |
| 401 | 40 | | | | | | | | | | 20 | 5 | citrate |
| 402 | 40 | | | | | | | | | | 100 | 5 | citrate |
| 403 | 50 | | | | | | 10 | | | | | 5 | citrate |
| 404 | 50 | | | | | | 20 | | | | | 5 | citrate |
| 405 | 50 | | | | | | 30 | | | | | 5 | citrate |
| 406 | 50 | | | | | | 40 | | | | | 5 | citrate |
| 407 | 50 | | | | | | 50 | | | | | 5 | citrate |
| 408 | 100 | | | | | | 20 | | | | | 5 | citrate |
| 409 | 100 | | | | | | 30 | | | | | 5 | citrate |
| 410 | 100 | | | | | | 40 | | | | | 5 | citrate |
| 411 | 100 | | | | | | 50 | | | | | 5 | citrate |
| 412 | 150 | | | | | | 20 | | | | | 5 | citrate |
| 413 | 150 | | | | | | 30 | | | | | 5 | citrate |
| 414 | 150 | | | | | | 40 | | | | | 5 | citrate |
| 415 | 150 | | | | | | 50 | | | | | 5 | citrate |
| 416 | 200 | | | | | | 30 | | | | | 5 | citrate |
| 417 | 200 | | | | | | 40 | | | | | 5 | citrate |
| 418 | 200 | | | | | | 50 | | | | | 5 | citrate |
| 419 | 250 | | | | | | 30 | | | | | 5 | citrate |
| 420 | 250 | | | | | | 40 | | | | | 5 | citrate |
| 421 | 250 | | | | | | 50 | | | | | 5 | citrate |
| 422 | 300 | | | | | | 30 | | | | | 5 | citrate |
| 423 | 300 | | | | | | 40 | | | | | 5 | citrate |
| 424 | 300 | | | | | | 50 | | | | | 5 | citrate |
| 425 | 350 | | | | | | 40 | | | | | 5 | citrate |
| 426 | 400 | | | | | | 50 | | | | | 5 | citrate |
| 427 | 400 | | | | | | 40 | | | | | 5 | citrate |
| 428 | 450 | | | | | | 50 | | | | | 5 | citrate |
| 429 | 500 | | | | | | 50 | | | | | 5 | citrate |
| 430 | 20 | 5 | | | | | | | | | | 6 | citrate |
| 431 | 20 | 10 | | | | | | | | | | 6 | citrate |
| 432 | 20 | 20 | | | | | | | | | | 6 | citrate |
| 433 | 20 | 30 | | | | | | | | | | 6 | citrate |
| 434 | 20 | | 5 | | | | | | | | | 6 | citrate |
| 435 | 20 | | 10 | | | | | | | | | 6 | citrate |
| 436 | 20 | | 20 | | | | | | | | | 6 | citrate |
| 437 | 20 | | 30 | | | | | | | | | 6 | citrate |
| 438 | 20 | | | 5 | | | | | | | | 6 | citrate |
| 439 | 20 | | | 10 | | | | | | | | 6 | citrate |
| 440 | 20 | | | 20 | | | | | | | | 6 | citrate |
| 441 | 20 | | | 30 | | | | | | | | 6 | citrate |
| 442 | 20 | | | | 5 | | | | | | | 6 | citrate |
| 443 | 20 | | | | 10 | | | | | | | 6 | citrate |
| 444 | 20 | | | | 20 | | | | | | | 6 | citrate |
| 445 | 20 | | | | 30 | | | | | | | 6 | citrate |
| 446 | 20 | | | | | 5 | | | | | | 6 | citrate |
| 447 | 20 | | | | | 10 | | | | | | 6 | citrate |
| 448 | 20 | | | | | 20 | | | | | | 6 | citrate |
| 449 | 20 | | | | | 30 | | | | | | 6 | citrate |
| 450 | 20 | | | | | | 5 | | | | | 6 | citrate |
| 451 | 20 | | | | | | 10 | | | | | 6 | citrate |
| 452 | 20 | | | | | | 20 | | | | | 6 | citrate |
| 453 | 20 | | | | | | 30 | | | | | 6 | citrate |
| 454 | 20 | | | | | | | 10 | | | | 6 | citrate |
| 455 | 20 | | | | | | | 20 | | | | 6 | citrate |
| 456 | 20 | | | | | | | 100 | | | | 6 | citrate |
| 457 | 20 | | | | | | | | 10 | | | 6 | citrate |
| 458 | 20 | | | | | | | | 20 | | | 6 | citrate |
| 459 | 20 | | | | | | | | 100 | | | 6 | citrate |
| 460 | 20 | | | | | | | | | 10 | | 6 | citrate |
| 461 | 20 | | | | | | | | | 20 | | 6 | citrate |
| 462 | 20 | | | | | | | | | 100 | | 6 | citrate |
| 463 | 20 | | | | | | | | | | 5 | 6 | citrate |
| 464 | 20 | | | | | | | | | | 10 | 6 | citrate |
| 465 | 20 | | | | | | | | | | 20 | 6 | citrate |
| 466 | 20 | | | | | | | | | | 100 | 6 | citrate |
| 467 | 30 | 5 | | | | | | | | | | 6 | citrate |
| 468 | 30 | 10 | | | | | | | | | | 6 | citrate |
| 469 | 30 | 20 | | | | | | | | | | 6 | citrate |
| 470 | 30 | 30 | | | | | | | | | | 6 | citrate |
| 471 | 30 | | 5 | | | | | | | | | 6 | citrate |
| 472 | 30 | | 10 | | | | | | | | | 6 | citrate |
| 473 | 30 | | 20 | | | | | | | | | 6 | citrate |
| 474 | 30 | | 30 | | | | | | | | | 6 | citrate |
| 475 | 30 | | | 5 | | | | | | | | 6 | citrate |
| 476 | 30 | | | 10 | | | | | | | | 6 | citrate |
| 477 | 30 | | | 20 | | | | | | | | 6 | citrate |
| 478 | 30 | | | 30 | | | | | | | | 6 | citrate |
| 479 | 30 | | | | 5 | | | | | | | 6 | citrate |
| 480 | 30 | | | | 10 | | | | | | | 6 | citrate |
| 481 | 30 | | | | 20 | | | | | | | 6 | citrate |
| 482 | 30 | | | | 30 | | | | | | | 6 | citrate |
| 483 | 30 | | | | | 5 | | | | | | 6 | citrate |
| 484 | 30 | | | | | 10 | | | | | | 6 | citrate |
| 485 | 30 | | | | | 20 | | | | | | 6 | citrate |
| 486 | 30 | | | | | 30 | | | | | | 6 | citrate |
| 487 | 30 | | | | | | 5 | | | | | 6 | citrate |
| 488 | 30 | | | | | | 10 | | | | | 6 | citrate |
| 489 | 30 | | | | | | 20 | | | | | 6 | citrate |
| 490 | 30 | | | | | | 30 | | | | | 6 | citrate |
| 491 | 30 | | | | | | | 10 | | | | 6 | citrate |
| 492 | 30 | | | | | | | 20 | | | | 6 | citrate |
| 493 | 30 | | | | | | | 100 | | | | 6 | citrate |
| 494 | 30 | | | | | | | | 10 | | | 6 | citrate |
| 495 | 30 | | | | | | | | 20 | | | 6 | citrate |
| 496 | 30 | | | | | | | | 100 | | | 6 | citrate |
| 497 | 30 | | | | | | | | | 10 | | 6 | citrate |
| 498 | 30 | | | | | | | | | 20 | | 6 | citrate |
| 499 | 30 | | | | | | | | | 100 | | 6 | citrate |
| 500 | 30 | | | | | | | | | | | 6 | citrate |
| 501 | 30 | | | | | | | | | | | 6 | citrate |
| 502 | 30 | | | | | | | | | | 5 | 6 | citrate |
| 503 | 30 | | | | | | | | | | 10 | 6 | citrate |
| 504 | 30 | | | | | | | | | | 20 | 6 | citrate |
| 505 | 30 | | | | | | | | | | 100 | 6 | citrate |
| 506 | 40 | 5 | | | | | | | | | | 6 | citrate |
| 507 | 40 | 10 | | | | | | | | | | 6 | citrate |
| 508 | 40 | 20 | | | | | | | | | | 6 | citrate |
| 509 | 40 | 30 | | | | | | | | | | 6 | citrate |
| 510 | 40 | | 5 | | | | | | | | | 6 | citrate |
| 511 | 40 | | 10 | | | | | | | | | 6 | citrate |
| 512 | 40 | | 20 | | | | | | | | | 6 | citrate |
| 513 | 40 | | 30 | | | | | | | | | 6 | citrate |
| 514 | 40 | | | 5 | | | | | | | | 6 | citrate |
| 515 | 40 | | | 10 | | | | | | | | 6 | citrate |
| 516 | 40 | | | 20 | | | | | | | | 6 | citrate |
| 517 | 40 | | | 30 | | | | | | | | 6 | citrate |
| 518 | 40 | | | | 5 | | | | | | | 6 | citrate |
| 519 | 40 | | | | 10 | | | | | | | 6 | citrate |
| 520 | 40 | | | | 20 | | | | | | | 6 | citrate |
| 521 | 40 | | | | 30 | | | | | | | 6 | citrate |
| 522 | 40 | | | | | 5 | | | | | | 6 | citrate |
| 523 | 40 | | | | | 10 | | | | | | 6 | citrate |
| 524 | 40 | | | | | 20 | | | | | | 6 | citrate |
| 525 | 40 | | | | | 30 | | | | | | 6 | citrate |
| 526 | 40 | | | | | | 5 | | | | | 6 | citrate |
| 527 | 40 | | | | | | 10 | | | | | 6 | citrate |
| 528 | 40 | | | | | | 20 | | | | | 6 | citrate |
| 529 | 40 | | | | | | 30 | | | | | 6 | citrate |
| 530 | 40 | | | | | | | 10 | | | | 6 | citrate |
| 531 | 40 | | | | | | | 20 | | | | 6 | citrate |
| 532 | 40 | | | | | | | 100 | | | | 6 | citrate |
| 533 | 40 | | | | | | | | 10 | | | 6 | citrate |
| 534 | 40 | | | | | | | | 20 | | | 6 | citrate |
| 535 | 40 | | | | | | | | 100 | | | 6 | citrate |
| 536 | 40 | | | | | | | | | 10 | | 6 | citrate |
| 537 | 40 | | | | | | | | | 20 | | 6 | citrate |
| 538 | 40 | | | | | | | | | 100 | | 6 | citrate |
| 539 | 40 | | | | | | | | | | 5 | 6 | citrate |
| 540 | 40 | | | | | | | | | | 10 | 6 | citrate |
| 541 | 40 | | | | | | | | | | 20 | 6 | citrate |
| 542 | 40 | | | | | | | | | | 100 | 6 | citrate |
| 543 | 50 | | | | | | 10 | | | | | 6 | citrate |
| 544 | 50 | | | | | | 20 | | | | | 6 | citrate |
| 545 | 50 | | | | | | 30 | | | | | 6 | citrate |
| 546 | 50 | | | | | | 40 | | | | | 6 | citrate |
| 547 | 50 | | | | | | 50 | | | | | 6 | citrate |
| 548 | 100 | | | | | | 20 | | | | | 6 | citrate |
| 549 | 100 | | | | | | 30 | | | | | 6 | citrate |
| 550 | 100 | | | | | | 40 | | | | | 6 | citrate |
| 551 | 100 | | | | | | 50 | | | | | 6 | citrate |
| 552 | 150 | | | | | | 20 | | | | | 6 | citrate |
| 553 | 150 | | | | | | 30 | | | | | 6 | citrate |
| 554 | 150 | | | | | | 40 | | | | | 6 | citrate |
| 555 | 150 | | | | | | 50 | | | | | 6 | citrate |
| 556 | 200 | | | | | | 30 | | | | | 6 | citrate |
| 557 | 200 | | | | | | 40 | | | | | 6 | citrate |
| 558 | 200 | | | | | | 50 | | | | | 6 | citrate |
| 559 | 250 | | | | | | 30 | | | | | 6 | citrate |
| 560 | 250 | | | | | | 40 | | | | | 6 | citrate |
| 561 | 250 | | | | | | 50 | | | | | 6 | citrate |
| 562 | 300 | | | | | | 30 | | | | | 6 | citrate |
| 563 | 300 | | | | | | 40 | | | | | 6 | citrate |
| 564 | 300 | | | | | | 50 | | | | | 6 | citrate |
| 565 | 350 | | | | | | 40 | | | | | 6 | citrate |
| 566 | 400 | | | | | | 50 | | | | | 6 | citrate |
| 567 | 400 | | | | | | 40 | | | | | 6 | citrate |
| 568 | 450 | | | | | | 50 | | | | | 6 | citrate |
| 569 | 500 | | | | | | 50 | | | | | 6 | citrate |
| 570 | 20 | 5 | | | | | | | | | | 5 | Tartrate |
| 571 | 20 | 10 | | | | | | | | | | 5 | Tartrate |
| 572 | 20 | 20 | | | | | | | | | | 5 | Tartrate |
| 573 | 20 | 30 | | | | | | | | | | 5 | Tartrate |
| 574 | 20 | | 5 | | | | | | | | | 5 | Tartrate |
| 575 | 20 | | 10 | | | | | | | | | 5 | Tartrate |
| 576 | 20 | | 20 | | | | | | | | | 5 | Tartrate |
| 577 | 20 | | 30 | | | | | | | | | 5 | Tartrate |
| 578 | 20 | | | 5 | | | | | | | | 5 | Tartrate |
| 579 | 20 | | | 10 | | | | | | | | 5 | Tartrate |
| 580 | 20 | | | 20 | | | | | | | | 5 | Tartrate |
| 581 | 20 | | | 30 | | | | | | | | 5 | Tartrate |
| 582 | 20 | | | | 5 | | | | | | | 5 | Tartrate |
| 583 | 20 | | | | 10 | | | | | | | 5 | Tartrate |
| 584 | 20 | | | | 20 | | | | | | | 5 | Tartrate |
| 585 | 20 | | | | 30 | | | | | | | 5 | Tartrate |
| 586 | 20 | | | | | 5 | | | | | | 5 | Tartrate |
| 587 | 20 | | | | | 10 | | | | | | 5 | Tartrate |
| 588 | 20 | | | | | 20 | | | | | | 5 | Tartrate |
| 589 | 20 | | | | | 30 | | | | | | 5 | Tartrate |
| 590 | 20 | | | | | | 5 | | | | | 5 | Tartrate |
| 591 | 20 | | | | | | 10 | | | | | 5 | Tartrate |
| 592 | 20 | | | | | | 20 | | | | | 5 | Tartrate |
| 593 | 20 | | | | | | 30 | | | | | 5 | Tartrate |
| 594 | 20 | | | | | | | 10 | | | | 5 | Tartrate |
| 595 | 20 | | | | | | | 20 | | | | 5 | Tartrate |
| 596 | 20 | | | | | | | 100 | | | | 5 | Tartrate |
| 597 | 20 | | | | | | | | 10 | | | 5 | Tartrate |
| 598 | 20 | | | | | | | | 20 | | | 5 | Tartrate |
| 599 | 20 | | | | | | | | 100 | | | 5 | Tartrate |
| 600 | 20 | | | | | | | | | 10 | | 5 | Tartrate |
| 601 | 20 | | | | | | | | | 20 | | 5 | Tartrate |
| 602 | 20 | | | | | | | | | 100 | | 5 | Tartrate |
| 603 | 20 | | | | | | | | | | 5 | 5 | Tartrate |
| 604 | 20 | | | | | | | | | | 10 | 5 | Tartrate |
| 605 | 20 | | | | | | | | | | 20 | 5 | Tartrate |
| 606 | 20 | | | | | | | | | | 100 | 5 | Tartrate |
| 607 | 30 | 5 | | | | | | | | | | 5 | Tartrate |
| 608 | 30 | 10 | | | | | | | | | | 5 | Tartrate |
| 609 | 30 | 20 | | | | | | | | | | 5 | Tartrate |
| 610 | 30 | 30 | | | | | | | | | | 5 | Tartrate |
| 611 | 30 | | 5 | | | | | | | | | 5 | Tartrate |
| 612 | 30 | | 10 | | | | | | | | | 5 | Tartrate |
| 613 | 30 | | 20 | | | | | | | | | 5 | Tartrate |
| 614 | 30 | | 30 | | | | | | | | | 5 | Tartrate |
| 615 | 30 | | | 5 | | | | | | | | 5 | Tartrate |
| 616 | 30 | | | 10 | | | | | | | | 5 | Tartrate |
| 617 | 30 | | | 20 | | | | | | | | 5 | Tartrate |
| 618 | 30 | | | 30 | | | | | | | | 5 | Tartrate |
| 619 | 30 | | | | 5 | | | | | | | 5 | Tartrate |
| 620 | 30 | | | | 10 | | | | | | | 5 | Tartrate |
| 621 | 30 | | | | 20 | | | | | | | 5 | Tartrate |
| 622 | 30 | | | | 30 | | | | | | | 5 | Tartrate |
| 623 | 30 | | | | | 5 | | | | | | 5 | Tartrate |
| 624 | 30 | | | | | 10 | | | | | | 5 | Tartrate |
| 625 | 30 | | | | | 20 | | | | | | 5 | Tartrate |
| 626 | 30 | | | | | 30 | | | | | | 5 | Tartrate |
| 627 | 30 | | | | | | 5 | | | | | 5 | Tartrate |
| 628 | 30 | | | | | | 10 | | | | | 5 | Tartrate |
| 629 | 30 | | | | | | 20 | | | | | 5 | Tartrate |
| 630 | 30 | | | | | | 30 | | | | | 5 | Tartrate |
| 631 | 30 | | | | | | | 10 | | | | 5 | Tartrate |
| 632 | 30 | | | | | | | 20 | | | | 5 | Tartrate |
| 633 | 30 | | | | | | | 100 | | | | 5 | Tartrate |
| 634 | 30 | | | | | | | | 10 | | | 5 | Tartrate |
| 635 | 30 | | | | | | | | 20 | | | 5 | Tartrate |
| 636 | 30 | | | | | | | | 100 | | | 5 | Tartrate |
| 637 | 30 | | | | | | | | | 10 | | 5 | Tartrate |
| 638 | 30 | | | | | | | | | 20 | | 5 | Tartrate |
| 639 | 30 | | | | | | | | | 100 | | 5 | Tartrate |
| 640 | 30 | | | | | | | | | | | 5 | Tartrate |
| 641 | 30 | | | | | | | | | | | 5 | Tartrate |
| 642 | 30 | | | | | | | | | | 5 | 5 | Tartrate |
| 643 | 30 | | | | | | | | | | 10 | 5 | Tartrate |
| 644 | 30 | | | | | | | | | | 20 | 5 | Tartrate |
| 645 | 30 | | | | | | | | | | 100 | 5 | Tartrate |
| 646 | 40 | 5 | | | | | | | | | | 5 | Tartrate |
| 647 | 40 | 10 | | | | | | | | | | 5 | Tartrate |
| 648 | 40 | 20 | | | | | | | | | | 5 | Tartrate |
| 649 | 40 | 30 | | | | | | | | | | 5 | Tartrate |
| 650 | 40 | | 5 | | | | | | | | | 5 | Tartrate |
| 651 | 40 | | 10 | | | | | | | | | 5 | Tartrate |
| 652 | 40 | | 20 | | | | | | | | | 5 | Tartrate |
| 653 | 40 | | 30 | | | | | | | | | 5 | Tartrate |
| 654 | 40 | | | 5 | | | | | | | | 5 | Tartrate |
| 655 | 40 | | | 10 | | | | | | | | 5 | Tartrate |
| 656 | 40 | | | 20 | | | | | | | | 5 | Tartrate |
| 657 | 40 | | | 30 | | | | | | | | 5 | Tartrate |
| 658 | 40 | | | | 5 | | | | | | | 5 | Tartrate |
| 659 | 40 | | | | 10 | | | | | | | 5 | Tartrate |
| 660 | 40 | | | | 20 | | | | | | | 5 | Tartrate |
| 661 | 40 | | | | 30 | | | | | | | 5 | Tartrate |
| 662 | 40 | | | | | 5 | | | | | | 5 | Tartrate |
| 663 | 40 | | | | | 10 | | | | | | 5 | Tartrate |
| 664 | 40 | | | | | 20 | | | | | | 5 | Tartrate |
| 665 | 40 | | | | | 30 | | | | | | 5 | Tartrate |
| 666 | 40 | | | | | | 5 | | | | | 5 | Tartrate |
| 667 | 40 | | | | | | 10 | | | | | 5 | Tartrate |
| 668 | 40 | | | | | | 20 | | | | | 5 | Tartrate |
| 669 | 40 | | | | | | 30 | | | | | 5 | Tartrate |
| 670 | 40 | | | | | | | 10 | | | | 5 | Tartrate |
| 671 | 40 | | | | | | | 20 | | | | 5 | Tartrate |
| 672 | 40 | | | | | | | 100 | | | | 5 | Tartrate |
| 673 | 40 | | | | | | | | 10 | | | 5 | Tartrate |
| 674 | 40 | | | | | | | | 20 | | | 5 | Tartrate |
| 675 | 40 | | | | | | | | 100 | | | 5 | Tartrate |
| 676 | 40 | | | | | | | | | 10 | | 5 | Tartrate |
| 677 | 40 | | | | | | | | | 20 | | 5 | Tartrate |
| 678 | 40 | | | | | | | | | 100 | | 5 | Tartrate |
| 679 | 40 | | | | | | | | | | 5 | 5 | Tartrate |
| 680 | 40 | | | | | | | | | | 10 | 5 | Tartrate |
| 681 | 40 | | | | | | | | | | 20 | 5 | Tartrate |
| 682 | 40 | | | | | | | | | | 100 | 5 | Tartrate |
| 683 | 50 | | | | | | 10 | | | | | 5 | Tartrate |
| 684 | 50 | | | | | | 20 | | | | | 5 | Tartrate |
| 685 | 50 | | | | | | 30 | | | | | 5 | Tartrate |
| 686 | 50 | | | | | | 40 | | | | | 5 | Tartrate |
| 687 | 50 | | | | | | 50 | | | | | 5 | Tartrate |
| 688 | 100 | | | | | | 20 | | | | | 5 | Tartrate |
| 689 | 100 | | | | | | 30 | | | | | 5 | Tartrate |
| 690 | 100 | | | | | | 40 | | | | | 5 | Tartrate |
| 691 | 100 | | | | | | 50 | | | | | 5 | Tartrate |
| 692 | 150 | | | | | | 20 | | | | | 5 | Tartrate |
| 693 | 150 | | | | | | 30 | | | | | 5 | Tartrate |
| 694 | 150 | | | | | | 40 | | | | | 5 | Tartrate |
| 695 | 150 | | | | | | 50 | | | | | 5 | Tartrate |
| 696 | 200 | | | | | | 30 | | | | | 5 | Tartrate |
| 697 | 200 | | | | | | 40 | | | | | 5 | Tartrate |
| 698 | 200 | | | | | | 50 | | | | | 5 | Tartrate |
| 699 | 250 | | | | | | 30 | | | | | 5 | Tartrate |
| 700 | 250 | | | | | | 40 | | | | | 5 | Tartrate |
| 701 | 250 | | | | | | 50 | | | | | 5 | Tartrate |
| 702 | 300 | | | | | | 30 | | | | | 5 | Tartrate |
| 703 | 300 | | | | | | 40 | | | | | 5 | Tartrate |
| 704 | 300 | | | | | | 50 | | | | | 5 | Tartrate |
| 705 | 350 | | | | | | 40 | | | | | 5 | Tartrate |
| 706 | 400 | | | | | | 50 | | | | | 5 | Tartrate |
| 707 | 400 | | | | | | 40 | | | | | 5 | Tartrate |
| 708 | 450 | | | | | | 50 | | | | | 5 | Tartrate |
| 709 | 500 | | | | | | 50 | | | | | 5 | Tartrate |
| 710 | 20 | 5 | | | | | | | | | | 6 | Tartrate |
| 711 | 20 | 10 | | | | | | | | | | 6 | Tartrate |
| 712 | 20 | 20 | | | | | | | | | | 6 | Tartrate |
| 713 | 20 | 30 | | | | | | | | | | 6 | Tartrate |
| 714 | 20 | | 5 | | | | | | | | | 6 | Tartrate |
| 715 | 20 | | 10 | | | | | | | | | 6 | Tartrate |
| 716 | 20 | | 20 | | | | | | | | | 6 | Tartrate |
| 717 | 20 | | 30 | | | | | | | | | 6 | Tartrate |
| 718 | 20 | | | 5 | | | | | | | | 6 | Tartrate |
| 719 | 20 | | | 10 | | | | | | | | 6 | Tartrate |
| 720 | 20 | | | 20 | | | | | | | | 6 | Tartrate |
| 721 | 20 | | | 30 | | | | | | | | 6 | Tartrate |
| 722 | 20 | | | | 5 | | | | | | | 6 | Tartrate |
| 723 | 20 | | | | 10 | | | | | | | 6 | Tartrate |
| 724 | 20 | | | | 20 | | | | | | | 6 | Tartrate |
| 725 | 20 | | | | 30 | | | | | | | 6 | Tartrate |
| 726 | 20 | | | | | 5 | | | | | | 6 | Tartrate |
| 727 | 20 | | | | | 10 | | | | | | 6 | Tartrate |
| 728 | 20 | | | | | 20 | | | | | | 6 | Tartrate |
| 729 | 20 | | | | | 30 | | | | | | 6 | Tartrate |
| 730 | 20 | | | | | | 5 | | | | | 6 | Tartrate |
| 731 | 20 | | | | | | 10 | | | | | 6 | Tartrate |
| 732 | 20 | | | | | | 20 | | | | | 6 | Tartrate |
| 733 | 20 | | | | | | 30 | | | | | 6 | Tartrate |
| 734 | 20 | | | | | | | 10 | | | | 6 | Tartrate |
| 735 | 20 | | | | | | | 20 | | | | 6 | Tartrate |
| 736 | 20 | | | | | | | 100 | | | | 6 | Tartrate |
| 737 | 20 | | | | | | | | 10 | | | 6 | Tartrate |
| 738 | 20 | | | | | | | | 20 | | | 6 | Tartrate |
| 739 | 20 | | | | | | | | 100 | | | 6 | Tartrate |
| 740 | 20 | | | | | | | | | 10 | | 6 | Tartrate |
| 741 | 20 | | | | | | | | | 20 | | 6 | Tartrate |
| 742 | 20 | | | | | | | | | 100 | | 6 | Tartrate |
| 743 | 20 | | | | | | | | | | 5 | 6 | Tartrate |
| 744 | 20 | | | | | | | | | | 10 | 6 | Tartrate |
| 745 | 20 | | | | | | | | | | 20 | 6 | Tartrate |
| 746 | 20 | | | | | | | | | | 100 | 6 | Tartrate |
| 747 | 30 | 5 | | | | | | | | | | 6 | Tartrate |
| 748 | 30 | 10 | | | | | | | | | | 6 | Tartrate |
| 749 | 30 | 20 | | | | | | | | | | 6 | Tartrate |
| 750 | 30 | 30 | | | | | | | | | | 6 | Tartrate |
| 751 | 30 | | 5 | | | | | | | | | 6 | Tartrate |
| 752 | 30 | | 10 | | | | | | | | | 6 | Tartrate |
| 753 | 30 | | 20 | | | | | | | | | 6 | Tartrate |
| 754 | 30 | | 30 | | | | | | | | | 6 | Tartrate |
| 755 | 30 | | | 5 | | | | | | | | 6 | Tartrate |
| 756 | 30 | | | 10 | | | | | | | | 6 | Tartrate |
| 757 | 30 | | | 20 | | | | | | | | 6 | Tartrate |
| 758 | 30 | | | 30 | | | | | | | | 6 | Tartrate |
| 759 | 30 | | | | 5 | | | | | | | 6 | Tartrate |
| 760 | 30 | | | | 10 | | | | | | | 6 | Tartrate |
| 761 | 30 | | | | 20 | | | | | | | 6 | Tartrate |
| 762 | 30 | | | | 30 | | | | | | | 6 | Tartrate |
| 763 | 30 | | | | | 5 | | | | | | 6 | Tartrate |
| 764 | 30 | | | | | 10 | | | | | | 6 | Tartrate |
| 765 | 30 | | | | | 20 | | | | | | 6 | Tartrate |
| 766 | 30 | | | | | 30 | | | | | | 6 | Tartrate |
| 767 | 30 | | | | | | 5 | | | | | 6 | Tartrate |
| 768 | 30 | | | | | | 10 | | | | | 6 | Tartrate |
| 769 | 30 | | | | | | 20 | | | | | 6 | Tartrate |
| 770 | 30 | | | | | | 30 | | | | | 6 | Tartrate |
| 771 | 30 | | | | | | | 10 | | | | 6 | Tartrate |
| 772 | 30 | | | | | | | 20 | | | | 6 | Tartrate |
| 773 | 30 | | | | | | | 100 | | | | 6 | Tartrate |
| 774 | 30 | | | | | | | | 10 | | | 6 | Tartrate |
| 775 | 30 | | | | | | | | 20 | | | 6 | Tartrate |
| 776 | 30 | | | | | | | | 100 | | | 6 | Tartrate |
| 777 | 30 | | | | | | | | | 10 | | 6 | Tartrate |
| 778 | 30 | | | | | | | | | 20 | | 6 | Tartrate |
| 779 | 30 | | | | | | | | | 100 | | 6 | Tartrate |
| 780 | 30 | | | | | | | | | | | 6 | Tartrate |
| 781 | 30 | | | | | | | | | | | 6 | Tartrate |
| 782 | 30 | | | | | | | | | | 5 | 6 | Tartrate |
| 783 | 30 | | | | | | | | | | 10 | 6 | Tartrate |
| 784 | 30 | | | | | | | | | | 20 | 6 | Tartrate |
| 785 | 30 | | | | | | | | | | 100 | 6 | Tartrate |
| 786 | 40 | 5 | | | | | | | | | | 6 | Tartrate |
| 787 | 40 | 10 | | | | | | | | | | 6 | Tartrate |
| 788 | 40 | 20 | | | | | | | | | | 6 | Tartrate |
| 789 | 40 | 30 | | | | | | | | | | 6 | Tartrate |
| 790 | 40 | | 5 | | | | | | | | | 6 | Tartrate |
| 791 | 40 | | 10 | | | | | | | | | 6 | Tartrate |
| 792 | 40 | | 20 | | | | | | | | | 6 | Tartrate |
| 793 | 40 | | 30 | | | | | | | | | 6 | Tartrate |
| 794 | 40 | | | 5 | | | | | | | | 6 | Tartrate |
| 795 | 40 | | | 10 | | | | | | | | 6 | Tartrate |
| 796 | 40 | | | 20 | | | | | | | | 6 | Tartrate |
| 797 | 40 | | | 30 | | | | | | | | 6 | Tartrate |
| 798 | 40 | | | | 5 | | | | | | | 6 | Tartrate |
| 799 | 40 | | | | 10 | | | | | | | 6 | Tartrate |
| 800 | 40 | | | | 20 | | | | | | | 6 | Tartrate |
| 801 | 40 | | | | 30 | | | | | | | 6 | Tartrate |
| 802 | 40 | | | | | 5 | | | | | | 6 | Tartrate |
| 803 | 40 | | | | | 10 | | | | | | 6 | Tartrate |
| 804 | 40 | | | | | 20 | | | | | | 6 | Tartrate |
| 805 | 40 | | | | | 30 | | | | | | 6 | Tartrate |
| 806 | 40 | | | | | | 5 | | | | | 6 | Tartrate |
| 807 | 40 | | | | | | 10 | | | | | 6 | Tartrate |
| 808 | 40 | | | | | | 20 | | | | | 6 | Tartrate |
| 809 | 40 | | | | | | 30 | | | | | 6 | Tartrate |
| 810 | 40 | | | | | | | 10 | | | | 6 | Tartrate |
| 811 | 40 | | | | | | | 20 | | | | 6 | Tartrate |
| 812 | 40 | | | | | | | 100 | | | | 6 | Tartrate |
| 813 | 40 | | | | | | | | 10 | | | 6 | Tartrate |
| 814 | 40 | | | | | | | | 20 | | | 6 | Tartrate |
| 815 | 40 | | | | | | | | 100 | | | 6 | Tartrate |
| 816 | 40 | | | | | | | | | 10 | | 6 | Tartrate |
| 817 | 40 | | | | | | | | | 20 | | 6 | Tartrate |
| 818 | 40 | | | | | | | | | 100 | | 6 | Tartrate |
| 819 | 40 | | | | | | | | | | 5 | 6 | Tartrate |
| 820 | 40 | | | | | | | | | | 10 | 6 | Tartrate |
| 821 | 40 | | | | | | | | | | 20 | 6 | Tartrate |
| 822 | 40 | | | | | | | | | | 100 | 6 | Tartrate |
| 823 | 50 | | | | | | 10 | | | | | 6 | Tartrate |
| 824 | 50 | | | | | | 20 | | | | | 6 | Tartrate |
| 825 | 50 | | | | | | 30 | | | | | 6 | Tartrate |
| 826 | 50 | | | | | | 40 | | | | | 6 | Tartrate |
| 827 | 50 | | | | | | 50 | | | | | 6 | Tartrate |
| 828 | 100 | | | | | | 20 | | | | | 6 | Tartrate |
| 829 | 100 | | | | | | 30 | | | | | 6 | Tartrate |
| 830 | 100 | | | | | | 40 | | | | | 6 | Tartrate |
| 831 | 100 | | | | | | 50 | | | | | 6 | Tartrate |
| 832 | 150 | | | | | | 20 | | | | | 6 | Tartrate |
| 833 | 150 | | | | | | 30 | | | | | 6 | Tartrate |
| 834 | 150 | | | | | | 40 | | | | | 6 | Tartrate |
| 835 | 150 | | | | | | 50 | | | | | 6 | Tartrate |
| 836 | 200 | | | | | | 30 | | | | | 6 | Tartrate |
| 837 | 200 | | | | | | 40 | | | | | 6 | Tartrate |
| 838 | 200 | | | | | | 50 | | | | | 6 | Tartrate |
| 839 | 250 | | | | | | 30 | | | | | 6 | Tartrate |
| 840 | 250 | | | | | | 40 | | | | | 6 | Tartrate |
| 841 | 250 | | | | | | 50 | | | | | 6 | Tartrate |
| 842 | 300 | | | | | | 30 | | | | | 6 | Tartrate |
| 843 | 300 | | | | | | 40 | | | | | 6 | Tartrate |
| 844 | 300 | | | | | | 50 | | | | | 6 | Tartrate |
| 845 | 350 | | | | | | 40 | | | | | 6 | Tartrate |
| 846 | 400 | | | | | | 50 | | | | | 6 | Tartrate |
| 847 | 400 | | | | | | 40 | | | | | 6 | Tartrate |
| 848 | 450 | | | | | | 50 | | | | | 6 | Tartrate |
| 849 | 500 | | | | | | 50 | | | | | 6 | Tartrate |
| 850 | 20 | 5 | | | | | | | | | | 5 | Lactate |
| 851 | 20 | 10 | | | | | | | | | | 5 | Lactate |
| 852 | 20 | 20 | | | | | | | | | | 5 | Lactate |
| 853 | 20 | 30 | | | | | | | | | | 5 | Lactate |
| 854 | 20 | | 5 | | | | | | | | | 5 | Lactate |
| 855 | 20 | | 10 | | | | | | | | | 5 | Lactate |
| 856 | 20 | | 20 | | | | | | | | | 5 | Lactate |
| 857 | 20 | | 30 | | | | | | | | | 5 | Lactate |
| 858 | 20 | | | 5 | | | | | | | | 5 | Lactate |
| 859 | 20 | | | 10 | | | | | | | | 5 | Lactate |
| 860 | 20 | | | 20 | | | | | | | | 5 | Lactate |
| 861 | 20 | | | 30 | | | | | | | | 5 | Lactate |
| 862 | 20 | | | | 5 | | | | | | | 5 | Lactate |
| 863 | 20 | | | | 10 | | | | | | | 5 | Lactate |
| 864 | 20 | | | | 20 | | | | | | | 5 | Lactate |
| 865 | 20 | | | | 30 | | | | | | | 5 | Lactate |
| 866 | 20 | | | | | 5 | | | | | | 5 | Lactate |
| 867 | 20 | | | | | 10 | | | | | | 5 | Lactate |
| 868 | 20 | | | | | 20 | | | | | | 5 | Lactate |
| 869 | 20 | | | | | 30 | | | | | | 5 | Lactate |
| 870 | 20 | | | | | | 5 | | | | | 5 | Lactate |
| 871 | 20 | | | | | | 10 | | | | | 5 | Lactate |
| 872 | 20 | | | | | | 20 | | | | | 5 | Lactate |
| 873 | 20 | | | | | | 30 | | | | | 5 | Lactate |
| 874 | 20 | | | | | | | 10 | | | | 5 | Lactate |
| 875 | 20 | | | | | | | 20 | | | | 5 | Lactate |
| 876 | 20 | | | | | | | 100 | | | | 5 | Lactate |
| 877 | 20 | | | | | | | | 10 | | | 5 | Lactate |
| 878 | 20 | | | | | | | | 20 | | | 5 | Lactate |
| 879 | 20 | | | | | | | | 100 | | | 5 | Lactate |
| 880 | 20 | | | | | | | | | 10 | | 5 | Lactate |
| 881 | 20 | | | | | | | | | 20 | | 5 | Lactate |
| 882 | 20 | | | | | | | | | 100 | | 5 | Lactate |
| 883 | 20 | | | | | | | | | | 5 | 5 | Lactate |
| 884 | 20 | | | | | | | | | | 10 | 5 | Lactate |
| 885 | 20 | | | | | | | | | | 20 | 5 | Lactate |
| 886 | 20 | | | | | | | | | | 100 | 5 | Lactate |
| 887 | 30 | 5 | | | | | | | | | | 5 | Lactate |
| 888 | 30 | 10 | | | | | | | | | | 5 | Lactate |
| 889 | 30 | 20 | | | | | | | | | | 5 | Lactate |
| 890 | 30 | 30 | | | | | | | | | | 5 | Lactate |
| 891 | 30 | | 5 | | | | | | | | | 5 | Lactate |
| 892 | 30 | | 10 | | | | | | | | | 5 | Lactate |
| 893 | 30 | | 20 | | | | | | | | | 5 | Lactate |
| 894 | 30 | | 30 | | | | | | | | | 5 | Lactate |
| 895 | 30 | | | 5 | | | | | | | | 5 | Lactate |
| 896 | 30 | | | 10 | | | | | | | | 5 | Lactate |
| 897 | 30 | | | 20 | | | | | | | | 5 | Lactate |
| 898 | 30 | | | 30 | | | | | | | | 5 | Lactate |
| 899 | 30 | | | | 5 | | | | | | | 5 | Lactate |
| 900 | 30 | | | | 10 | | | | | | | 5 | Lactate |
| 901 | 30 | | | | 20 | | | | | | | 5 | Lactate |
| 902 | 30 | | | | 30 | | | | | | | 5 | Lactate |
| 903 | 30 | | | | | 5 | | | | | | 5 | Lactate |
| 904 | 30 | | | | | 10 | | | | | | 5 | Lactate |
| 905 | 30 | | | | | 20 | | | | | | 5 | Lactate |
| 906 | 30 | | | | | 30 | | | | | | 5 | Lactate |
| 907 | 30 | | | | | | 5 | | | | | 5 | Lactate |
| 908 | 30 | | | | | | 10 | | | | | 5 | Lactate |
| 909 | 30 | | | | | | 20 | | | | | 5 | Lactate |
| 910 | 30 | | | | | | 30 | | | | | 5 | Lactate |
| 911 | 30 | | | | | | | 10 | | | | 5 | Lactate |
| 912 | 30 | | | | | | | 20 | | | | 5 | Lactate |
| 913 | 30 | | | | | | | 100 | | | | 5 | Lactate |
| 914 | 30 | | | | | | | | 10 | | | 5 | Lactate |
| 915 | 30 | | | | | | | | 20 | | | 5 | Lactate |
| 916 | 30 | | | | | | | | 100 | | | 5 | Lactate |
| 917 | 30 | | | | | | | | | 10 | | 5 | Lactate |
| 918 | 30 | | | | | | | | | 20 | | 5 | Lactate |
| 919 | 30 | | | | | | | | | 100 | | 5 | Lactate |
| 920 | 30 | | | | | | | | | | | 5 | Lactate |
| 921 | 30 | | | | | | | | | | | 5 | Lactate |
| 922 | 30 | | | | | | | | | | 5 | 5 | Lactate |
| 923 | 30 | | | | | | | | | | 10 | 5 | Lactate |
| 924 | 30 | | | | | | | | | | 20 | 5 | Lactate |
| 925 | 30 | | | | | | | | | | 100 | 5 | Lactate |
| 926 | 40 | 5 | | | | | | | | | | 5 | Lactate |
| 927 | 40 | 10 | | | | | | | | | | 5 | Lactate |
| 928 | 40 | 20 | | | | | | | | | | 5 | Lactate |
| 929 | 40 | 30 | | | | | | | | | | 5 | Lactate |
| 930 | 40 | | 5 | | | | | | | | | 5 | Lactate |
| 931 | 40 | | 10 | | | | | | | | | 5 | Lactate |
| 932 | 40 | | 20 | | | | | | | | | 5 | Lactate |
| 933 | 40 | | 30 | | | | | | | | | 5 | Lactate |
| 934 | 40 | | | 5 | | | | | | | | 5 | Lactate |
| 935 | 40 | | | 10 | | | | | | | | 5 | Lactate |
| 936 | 40 | | | 20 | | | | | | | | 5 | Lactate |
| 937 | 40 | | | 30 | | | | | | | | 5 | Lactate |
| 938 | 40 | | | | 5 | | | | | | | 5 | Lactate |
| 939 | 40 | | | | 10 | | | | | | | 5 | Lactate |
| 940 | 40 | | | | 20 | | | | | | | 5 | Lactate |
| 941 | 40 | | | | 30 | | | | | | | 5 | Lactate |
| 942 | 40 | | | | | 5 | | | | | | 5 | Lactate |
| 943 | 40 | | | | | 10 | | | | | | 5 | Lactate |
| 944 | 40 | | | | | 20 | | | | | | 5 | Lactate |
| 945 | 40 | | | | | 30 | | | | | | 5 | Lactate |
| 946 | 40 | | | | | | 5 | | | | | 5 | Lactate |
| 947 | 40 | | | | | | 10 | | | | | 5 | Lactate |
| 948 | 40 | | | | | | 20 | | | | | 5 | Lactate |
| 949 | 40 | | | | | | 30 | | | | | 5 | Lactate |
| 950 | 40 | | | | | | | 10 | | | | 5 | Lactate |
| 951 | 40 | | | | | | | 20 | | | | 5 | Lactate |
| 952 | 40 | | | | | | | 100 | | | | 5 | Lactate |
| 953 | 40 | | | | | | | | 10 | | | 5 | Lactate |
| 954 | 40 | | | | | | | | 20 | | | 5 | Lactate |
| 955 | 40 | | | | | | | | 100 | | | 5 | Lactate |
| 956 | 40 | | | | | | | | | 10 | | 5 | Lactate |
| 957 | 40 | | | | | | | | | 20 | | 5 | Lactate |
| 958 | 40 | | | | | | | | | 100 | | 5 | Lactate |
| 959 | 40 | | | | | | | | | | 5 | 5 | Lactate |
| 960 | 40 | | | | | | | | | | 10 | 5 | Lactate |
| 961 | 40 | | | | | | | | | | 20 | 5 | Lactate |
| 962 | 40 | | | | | | | | | | 100 | 5 | Lactate |
| 963 | 50 | | | | | | 10 | | | | | 5 | Lactate |
| 964 | 50 | | | | | | 20 | | | | | 5 | Lactate |
| 965 | 50 | | | | | | 30 | | | | | 5 | Lactate |
| 966 | 50 | | | | | | 40 | | | | | 5 | Lactate |
| 967 | 50 | | | | | | 50 | | | | | 5 | Lactate |
| 968 | 100 | | | | | | 20 | | | | | 5 | Lactate |
| 969 | 100 | | | | | | 30 | | | | | 5 | Lactate |
| 970 | 100 | | | | | | 40 | | | | | 5 | Lactate |
| 971 | 100 | | | | | | 50 | | | | | 5 | Lactate |
| 972 | 150 | | | | | | 20 | | | | | 5 | Lactate |
| 973 | 150 | | | | | | 30 | | | | | 5 | Lactate |
| 974 | 150 | | | | | | 40 | | | | | 5 | Lactate |
| 975 | 150 | | | | | | 50 | | | | | 5 | Lactate |
| 976 | 200 | | | | | | 30 | | | | | 5 | Lactate |
| 977 | 200 | | | | | | 40 | | | | | 5 | Lactate |
| 978 | 200 | | | | | | 50 | | | | | 5 | Lactate |
| 979 | 250 | | | | | | 30 | | | | | 5 | Lactate |
| 980 | 250 | | | | | | 40 | | | | | 5 | Lactate |
| 981 | 250 | | | | | | 50 | | | | | 5 | Lactate |
| 982 | 300 | | | | | | 30 | | | | | 5 | Lactate |
| 983 | 300 | | | | | | 40 | | | | | 5 | Lactate |
| 984 | 300 | | | | | | 50 | | | | | 5 | Lactate |
| 985 | 350 | | | | | | 40 | | | | | 5 | Lactate |
| 986 | 400 | | | | | | 50 | | | | | 5 | Lactate |
| 987 | 400 | | | | | | 40 | | | | | 5 | Lactate |
| 988 | 450 | | | | | | 50 | | | | | 5 | Lactate |
| 989 | 500 | | | | | | 50 | | | | | 5 | Lactate |
| 990 | 20 | 5 | | | | | | | | | | 6 | Tartrate |
| 991 | 20 | 10 | | | | | | | | | | 6 | Tartrate |
| 992 | 20 | 20 | | | | | | | | | | 6 | Tartrate |
| 993 | 20 | 30 | | | | | | | | | | 6 | Tartrate |
| 994 | 20 | | 5 | | | | | | | | | 6 | Tartrate |
| 995 | 20 | | 10 | | | | | | | | | 6 | Tartrate |
| 996 | 20 | | 20 | | | | | | | | | 6 | Tartrate |
| 997 | 20 | | 30 | | | | | | | | | 6 | Tartrate |
| 998 | 20 | | | 5 | | | | | | | | 6 | Tartrate |
| 999 | 20 | | | 10 | | | | | | | | 6 | Tartrate |
| 1000 | 20 | | | 20 | | | | | | | | 6 | Tartrate |
| 1001 | 20 | | | 30 | | | | | | | | 6 | Tartrate |
| 1002 | 20 | | | | 5 | | | | | | | 6 | Tartrate |
| 1003 | 20 | | | | 10 | | | | | | | 6 | Tartrate |
| 1004 | 20 | | | | 20 | | | | | | | 6 | Tartrate |
| 1005 | 20 | | | | 30 | | | | | | | 6 | Tartrate |
| 1006 | 20 | | | | | 5 | | | | | | 6 | Tartrate |
| 1007 | 20 | | | | | 10 | | | | | | 6 | Tartrate |
| 1008 | 20 | | | | | 20 | | | | | | 6 | Tartrate |
| 1009 | 20 | | | | | 30 | | | | | | 6 | Tartrate |
| 1010 | 20 | | | | | | 5 | | | | | 6 | Tartrate |
| 1011 | 20 | | | | | | 10 | | | | | 6 | Tartrate |
| 1012 | 20 | | | | | | 20 | | | | | 6 | Tartrate |
| 1013 | 20 | | | | | | 30 | | | | | 6 | Tartrate |
| 1014 | 20 | | | | | | | 10 | | | | 6 | Tartrate |
| 1015 | 20 | | | | | | | 20 | | | | 6 | Tartrate |
| 1016 | 20 | | | | | | | 100 | | | | 6 | Tartrate |
| 1017 | 20 | | | | | | | | 10 | | | 6 | Tartrate |
| 1018 | 20 | | | | | | | | 20 | | | 6 | Tartrate |
| 1019 | 20 | | | | | | | | 100 | | | 6 | Tartrate |
| 1020 | 20 | | | | | | | | | 10 | | 6 | Tartrate |
| 1021 | 20 | | | | | | | | | 20 | | 6 | Tartrate |
| 1022 | 20 | | | | | | | | | 100 | | 6 | Tartrate |
| 1023 | 20 | | | | | | | | | | 5 | 6 | Tartrate |
| 1024 | 20 | | | | | | | | | | 10 | 6 | Tartrate |
| 1025 | 20 | | | | | | | | | | 20 | 6 | Tartrate |
| 1026 | 20 | | | | | | | | | | 100 | 6 | Tartrate |
| 1027 | 30 | 5 | | | | | | | | | | 6 | Tartrate |
| 1028 | 30 | 10 | | | | | | | | | | 6 | Tartrate |
| 1029 | 30 | 20 | | | | | | | | | | 6 | Tartrate |
| 1030 | 30 | 30 | | | | | | | | | | 6 | Tartrate |
| 1031 | 30 | | 5 | | | | | | | | | 6 | Tartrate |
| 1032 | 30 | | 10 | | | | | | | | | 6 | Tartrate |
| 1033 | 30 | | 20 | | | | | | | | | 6 | Tartrate |
| 1034 | 30 | | 30 | | | | | | | | | 6 | Tartrate |
| 1035 | 30 | | | 5 | | | | | | | | 6 | Tartrate |
| 1036 | 30 | | | 10 | | | | | | | | 6 | Tartrate |
| 1037 | 30 | | | 20 | | | | | | | | 6 | Tartrate |
| 1038 | 30 | | | 30 | | | | | | | | 6 | Tartrate |
| 1039 | 30 | | | | 5 | | | | | | | 6 | Tartrate |
| 1040 | 30 | | | | 10 | | | | | | | 6 | Tartrate |
| 1041 | 30 | | | | 20 | | | | | | | 6 | Tartrate |
| 1042 | 30 | | | | 30 | | | | | | | 6 | Tartrate |
| 1043 | 30 | | | | | 5 | | | | | | 6 | Tartrate |
| 1044 | 30 | | | | | 10 | | | | | | 6 | Tartrate |
| 1045 | 30 | | | | | 20 | | | | | | 6 | Tartrate |
| 1046 | 30 | | | | | 30 | | | | | | 6 | Tartrate |
| 1047 | 30 | | | | | | 5 | | | | | 6 | Tartrate |
| 1048 | 30 | | | | | | 10 | | | | | 6 | Tartrate |
| 1049 | 30 | | | | | | 20 | | | | | 6 | Tartrate |
| 1050 | 30 | | | | | | 30 | | | | | 6 | Tartrate |
| 1051 | 30 | | | | | | | 10 | | | | 6 | Tartrate |
| 1052 | 30 | | | | | | | 20 | | | | 6 | Tartrate |
| 1053 | 30 | | | | | | | 100 | | | | 6 | Tartrate |
| 1054 | 30 | | | | | | | | 10 | | | 6 | Tartrate |
| 1055 | 30 | | | | | | | | 20 | | | 6 | Tartrate |
| 1056 | 30 | | | | | | | | 100 | | | 6 | Tartrate |
| 1057 | 30 | | | | | | | | | 10 | | 6 | Tartrate |
| 1058 | 30 | | | | | | | | | 20 | | 6 | Tartrate |
| 1059 | 30 | | | | | | | | | 100 | | 6 | Tartrate |
| 1060 | 30 | | | | | | | | | | | 6 | Tartrate |
| 1061 | 30 | | | | | | | | | | | 6 | Tartrate |
| 1062 | 30 | | | | | | | | | | 5 | 6 | Tartrate |
| 1063 | 30 | | | | | | | | | | 10 | 6 | Tartrate |
| 1064 | 30 | | | | | | | | | | 20 | 6 | Tartrate |
| 1065 | 30 | | | | | | | | | | 100 | 6 | Tartrate |
| 1066 | 40 | 5 | | | | | | | | | | 6 | Tartrate |
| 1067 | 40 | 10 | | | | | | | | | | 6 | Tartrate |
| 1068 | 40 | 20 | | | | | | | | | | 6 | Tartrate |
| 1069 | 40 | 30 | | | | | | | | | | 6 | Tartrate |
| 1070 | 40 | | 5 | | | | | | | | | 6 | Tartrate |
| 1071 | 40 | | 10 | | | | | | | | | 6 | Tartrate |
| 1072 | 40 | | 20 | | | | | | | | | 6 | Tartrate |
| 1073 | 40 | | 30 | | | | | | | | | 6 | Tartrate |
| 1074 | 40 | | | 5 | | | | | | | | 6 | Tartrate |
| 1075 | 40 | | | 10 | | | | | | | | 6 | Tartrate |
| 1076 | 40 | | | 20 | | | | | | | | 6 | Tartrate |
| 1077 | 40 | | | 30 | | | | | | | | 6 | Tartrate |
| 1078 | 40 | | | | 5 | | | | | | | 6 | Tartrate |
| 1079 | 40 | | | | 10 | | | | | | | 6 | Tartrate |
| 1080 | 40 | | | | 20 | | | | | | | 6 | Tartrate |
| 1081 | 40 | | | | 30 | | | | | | | 6 | Tartrate |
| 1082 | 40 | | | | | 5 | | | | | | 6 | Tartrate |
| 1083 | 40 | | | | | 10 | | | | | | 6 | Tartrate |
| 1084 | 40 | | | | | 20 | | | | | | 6 | Tartrate |
| 1085 | 40 | | | | | 30 | | | | | | 6 | Tartrate |
| 1086 | 40 | | | | | | 5 | | | | | 6 | Tartrate |
| 1087 | 40 | | | | | | 10 | | | | | 6 | Tartrate |
| 1088 | 40 | | | | | | 20 | | | | | 6 | Tartrate |
| 1089 | 40 | | | | | | 30 | | | | | 6 | Tartrate |
| 1090 | 40 | | | | | | | 10 | | | | 6 | Tartrate |
| 1091 | 40 | | | | | | | 20 | | | | 6 | Tartrate |
| 1092 | 40 | | | | | | | 100 | | | | 6 | Tartrate |
| 1093 | 40 | | | | | | | | 10 | | | 6 | Tartrate |
| 1094 | 40 | | | | | | | | 20 | | | 6 | Tartrate |
| 1095 | 40 | | | | | | | | 100 | | | 6 | Tartrate |
| 1096 | 40 | | | | | | | | | 10 | | 6 | Tartrate |
| 1097 | 40 | | | | | | | | | 20 | | 6 | Tartrate |
| 1098 | 40 | | | | | | | | | 100 | | 6 | Tartrate |
| 1099 | 40 | | | | | | | | | | 5 | 6 | Tartrate |
| 1100 | 40 | | | | | | | | | | 10 | 6 | Tartrate |
| 1101 | 40 | | | | | | | | | | 20 | 6 | Tartrate |
| 1102 | 40 | | | | | | | | | | 100 | 6 | Tartrate |
| 1103 | 50 | | | | | | 10 | | | | | 6 | Tartrate |
| 1104 | 50 | | | | | | 20 | | | | | 6 | Tartrate |
| 1105 | 50 | | | | | | 30 | | | | | 6 | Tartrate |
| 1106 | 50 | | | | | | 40 | | | | | 6 | Tartrate |
| 1107 | 50 | | | | | | 50 | | | | | 6 | Tartrate |
| 1108 | 100 | | | | | | 20 | | | | | 6 | Tartrate |
| 1109 | 100 | | | | | | 30 | | | | | 6 | Tartrate |
| 1110 | 100 | | | | | | 40 | | | | | 6 | Tartrate |
| 1111 | 100 | | | | | | 50 | | | | | 6 | Tartrate |
| 1112 | 150 | | | | | | 20 | | | | | 6 | Tartrate |
| 1113 | 150 | | | | | | 30 | | | | | 6 | Tartrate |
| 1114 | 150 | | | | | | 40 | | | | | 6 | Tartrate |
| 1115 | 150 | | | | | | 50 | | | | | 6 | Tartrate |
| 1116 | 200 | | | | | | 30 | | | | | 6 | Tartrate |
| 1117 | 200 | | | | | | 40 | | | | | 6 | Tartrate |
| 1118 | 200 | | | | | | 50 | | | | | 6 | Tartrate |
| 1119 | 250 | | | | | | 30 | | | | | 6 | Tartrate |
| 1120 | 250 | | | | | | 40 | | | | | 6 | Tartrate |
| 1121 | 250 | | | | | | 50 | | | | | 6 | Tartrate |
| 1122 | 300 | | | | | | 30 | | | | | 6 | Tartrate |
| 1123 | 300 | | | | | | 40 | | | | | 6 | Tartrate |
| 1124 | 300 | | | | | | 50 | | | | | 6 | Tartrate |
| 1125 | 350 | | | | | | 40 | | | | | 6 | Tartrate |
| 1126 | 400 | | | | | | 50 | | | | | 6 | Tartrate |
| 1127 | 400 | | | | | | 40 | | | | | 6 | Tartrate |
| 1128 | 450 | | | | | | 50 | | | | | 6 | Tartrate |
| 1129 | 500 | | | | | | 50 | | | | | 6 | Tartrate |

### Example 3: Solubility Studies with Hydroxypropyl β Cyclodextrin (HPβCD)

This example illustrates solubility properties of certain formulations of imatinib free base in liquid solution.

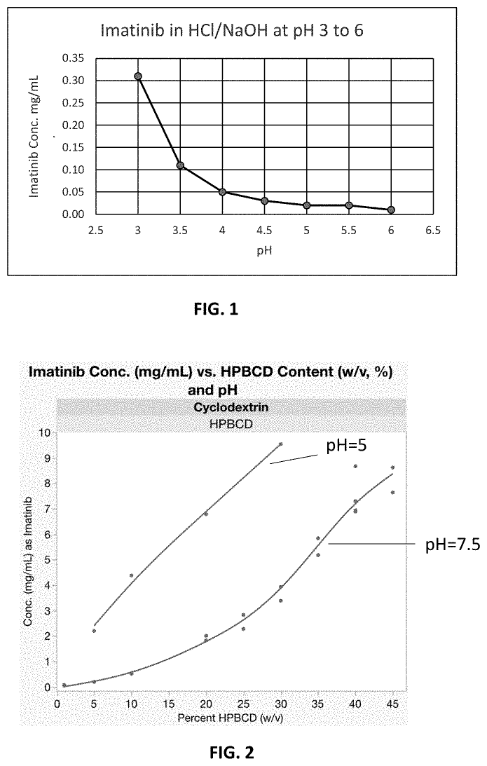

Imatinib free base was poorly soluble in water at physiologically relevant pH range of 4-8 (FIG. 1). FIG. 1 displays the maximum concentration of imatinib free base (mg/mL) as a function of pH. As seen in FIG. 1, a lower pH improved the solubility of imatinib free base. At pH of 3, the concentration of imatinib free base was around 0.30 mg/mL. At pH of 6, the concentration of imatinib free base was <0.01 mg/mL.

### Example 4: Solubility Studies with HPβCD and pH

This example illustrates solubility properties of certain formulations of imatinib free base in liquid solution.

FIG. 2 displays the maximum concentration of imatinib free base as a function of percent HPβCD at pH of 5 and 7.5. An improved solubility of imatinib free base was shown at a pH of 7.5. At 45% HPβCD (w/v), the amount of imatinib free base dissolved was 8.7 mg/mL. The addition of HPβCD improved solubility, for instance, the maximum concentration of imatinib free base was increased from <0.01 mg/mL at 0% HPβCD to 8.7 mg/mL to 45% HPβCD, respectively. At a pH of 5 and about 5% HPβCD (w/v), the concentration of imatinib free base dissolved was around 2 mg/mL. At a pH of 5 and between 25-30% HPβCD (w/v), the concentration of imatinib free base dissolved was 9.5 mg/mL. Decreasing the pH improved the solubility of imatinib free base for all concentrations tested. The decrease of pH from 7.5 to 5.0 provided 2.5 to 5.8 mg/mL concentration increase with HPβCD. The maximum imatinib free base concentration observed was 9.5 mg/mL.

### Example 5: Solubility Studies with HPβCD or Sulfobutylether βCyclodextrin (SBEβCD) and pH

This example illustrates solubility properties of certain exemplary formulations of imatinib free base in liquid solution.

FIG. 3A displays the maximum concentration of imatinib free base as a function of percent cyclodextrin (HPβCD or sulfobutylether β cyclodextrin (SBEβCD)) at different pH levels. The relationship between solubility of imatinib free base and the concentration of HPβCD (w/v) in the solution was tested at a pH of 5 and 7.5. The relationship between solubility of imatinib free base and the concentration of SBEβCD (w/v) in the solution was tested at a pH of 5. At a pH of 5 and about 5% HPβCD (w/v), the maximum concentration of imatinib free base dissolved was around 2 mg/mL. At a pH of 5 and between 25-30% HPβCD (w/v), the concentration of imatinib free base dissolved was 9.5 mg/mL. At a pH of 5 and 10% SBEβCD (w/v), the concentration of imatinib free base dissolved was about 70 mg/mL. The results showed about 3500× solubility increase compared to imatinib free base in pH 5 water, which was 0.02 mg/mL. The data showed about 16× solubility increase compared to 10% HPβCD (w/v) at pH 5, which was 4.4 mg/mL. The molar ratio of (imatinib/SBEβCD) in the solution was 3.1. At a pH of 5 and 20% SBEβCD (w/v), the concentration of imatinib free base dissolved was about 160 mg/mL. The results showed at least about 8000× solubility increase compared to imatinib free base in pH 5 water. The data showed about 25× solubility increase compared to 20% HPβCD (w/v) at pH 5, which was 6.8 mg/mL. The molar ratio of (imatinib/SBEβCD) in the solution was 3.5.

Further, at a pH of 5 and 25% SBEβCD (w/v), the concentration of imatinib free base dissolved was about 200 mg/mL. At a pH of 5 and 30% SBEβCD (w/v), the concentration of imatinib free base dissolved was about 225 mg/mL.

FIG. 3B shows pictures of exemplary imatinib solutions and suspension under different pH conditions. About 30 mg/mL imatinib free base was mixed with three different vehicles (30% SBβCD in 50 mM phosphate buffer adjusted at three different pH levels: 7, 5, and 3). As shown by the pictures, at pH=7, white suspension was obtained, while clear solutions were obtained under pH of 5 and pH of 3. This data suggest that solubility of imatinib free base in aqueous solution containing SBβCD can be pH dependent, e.g., the low the pH of the SBβCD aqueous solution is, the higher the solubility of imatinib free base in the solution.

### Example 6: Solubility Studies with SBEβCD and Dilution with Water

This example illustrates the effect of dilution with water on the solubility of imatinib free base in cyclodextrin-based formulation.

In one experiment, solubility of imatinib free base in an exemplary imatinib free base formulation upon dilution in water was examined. Briefly, the exemplary imatinib free base solution (50 mg/mL imatinib free base, 10% SBEβCD, pH 5.1) was diluted 40 times in water for injection (WFI). Upon dilution, imatinib precipitated. The pH of the final suspension after dilution was measured as 5.4. Since imatinib is still predominately protonated at pH of 5.4, the observed precipitation of imatinib may not be a pH-dependent effect.

In another experiment, another exemplary imatinib solution (100 mg/mL imatinib free base, 15% w/v SBEβCD, 50 mM phosphate buffer, pH 5.0) was diluted 20 times in water. Upon dilution, imatinib also precipitated. The resulting suspension was used in the intratracheal (IT) arm of the rat pharmacokinetic study shown in Example 9 below.

### Example 7: Solubility Studies with SBEβCD and Dilution

This example examines the effect of dilution while keeping pH constant on the solubility of imatinib free base in cyclodextrin-based formulation.

In one experiment, solubility of imatinib free base in exemplary imatinib free base formulations upon dilution while pH is kept constant is tested. Briefly, each of various exemplary imatinib free base solutions (100 mL in volume, saturated imatinib free base, different SBβCD concentrations, pH 5) is diluted with phosphate buffer (50 mM, pH 5) to arrive at a formulation with a final SBEβCD concentration of 1.5% (w/v). The solubility of imatinib free base in the SBEβCD solution is predicted to decrease upon dilution despite the pH being constant, based on the measurement as shown in Example 5 and FIG. 3A. Table 4 summarizes the predicted precipitation of imatinib free base in each solution upon dilution to yield a final SBEβCD concentration of 1.5% (w/v).

TABLE 4
Predicted precipitation of imatinib free base upon dilution

| | | | | Concentration | | | |
|---|---|---|---|---|---|---|---|
| | | Dissolved | | Imatinib | Dissolved | | |
| | | Imatinib | | Free Base | Imatinib | Precipitated | |
| Imatinib | Percent | Free Base | | Concentration | Free Base | Imatinib | Percent |
| Freebase | SBEβCD | Before Dilution | Dilution | Upon Dilution | Upon Dilution | Free Base | Precipitation |
| (mg/mL) | (w/v) | (mg) | Factor | (mg/mL) | (mg) | (mg) | (%)* |
| 5 | 1.5 | 500 | n/a | 5 | 500 | 0 | 0 |
| 17.5 | 5 | 1750 | 3.3 | | 1650 | 100 | 5.7 |
| 71.5 | 10 | 7150 | 6.7 | | 3350 | 3800 | 53 |
| 122 | 15 | 12200 | 10.0 | | 5000 | 7200 | 59 |
| 160 | 20 | 16000 | 13.3 | | 6650 | 9350 | 58 |
| 197 | 25 | 19700 | 16.7 | | 8350 | 11350 | 58 |
| 227 | 30 | 22700 | 20.0 | | 10000 | 12700 | 56 |

* Percent Precipitation = 100* (total imatinib − dissolved imatinib)/total imatinib

### Example 8: Taste Test of Sulfobutylether β Cyclodextrin (SBEβCD)-Based Formulation

This example illustrates organoleptic properties of certain exemplary formulations of imatinib free base in liquid solution.

Tester 5 (T5) tested inhaling two formulations:
a. 40 mg/mL imatinib, 10% w/v SBEBCD, 50 mM phosphate buffer (pH 5)
b. 80 mg/mL imatinib, 20% w/v SBEBCD, 50 mM phosphate buffer (pH 5).

T5 did not cough after inhalation of each of the two formulations for about 2 minutes (continuous inhalations). T5 did not experience any cough, any desire to cough, or throat irritation.

T5 stated the imatinib free base formulation with SBEβCD resulted in no coughing (adverse reaction). The same tester T5 also tasted imatinib mesylate formulations and coughed severely when inhaling the imatinib mesylate formulations.

### Example 9: Test of Exemplary Formulation in Rats

This example illustrates and compares pharmacokinetics of an exemplary imatinib free base/cyclodextrin formulation (“imatinib free base”) and imatinib mesylate formulation (“imatinib mesylate”) in rats following intravenously (IV) and intratracheal (IT) administration, respectively.

It was found that a concentration of imatinib free base compatible with IT rat dosing was 5 mg/mL. It was observed, however, exemplary imatinib free base formulation (100 mg/mL imatinib free base, 15% w/v sulfobutylether β cyclodextrin (SBEβCD), 50 mM phosphate buffer, pH 5.0) precipitated upon dilution, thus the 100 mg/mL imatinib freebase formulation was diluted 20-fold in water immediately prior to dosing and administered IT as a 5 mg/mL suspension. 6 mg/mL imatinib mesylate (equivalent to 5 mg/mL imatinib free base) solution formulation was used as a comparator in the IV arm of the study. Both groups received a single dose equivalent to 1 mg/kg dose imatinib free base. Each group had four animals euthanized at 6 time points post administration for sample collections 1-3, 5, 10, 20, 30, or 60 minutes. Lung tissue and blood were collected for analysis.

All animals survived to their scheduled necropsy. No abnormal clinical observations were noted throughout the study. No test article related changes in body weight were observed during the study. In general, animal necropsy results were grossly unremarkable.

Materials and Methods

TABLE 5
Test Article Formulation R1: Imatinib mesylate

| | |
|---|---|
| Identity: | Imatinib mesylate |
| Description: | 6 mg/mL imatinib mesylate (containing 5 mg/mL |
| | imatinib free base) solution, pH 5.0 |
| Preparation: | Not applicable. Test article used as received. |
| Supplier/Manufacturer: | Nitto Avecia Pharma Services |
| Lot: | 4389-166-2 |
| Storage Conditions: | Room temperature (ambient) |

TABLE 6
Test Article Formulation R2: Imatinib free base/cyclodextrin

| | |
|---|---|
| Identity: | Imatinib free base |
| Description: | 5 mg/mL imatinib free base suspension, 0.75% |
| | w/v sulfobutylether β cyclodextrin, 2.5 mM |
| | phosphate buffer, pH 5.0 |
| Preparation : | Test article was prepared as a suspension |
| | immediately prior to administration by |
| | diluting a 100 mg/mL imatinib free base, |
| | 15% w/v sulfobutylether-b-cyclodextrin, |
| | 50 mM phosphate buffer, pH 5.0 solution |
| | 20x in water. |
| Supplier/Manufacturer : | Nitto Avecia Pharma Services |
| Lot: | 4389-166-1 |
| Storage Conditions: | Room temperature (ambient) |

Test System: 72 male Sprague Dawley rats (Charles River Laboratories) were used for the study, 8 weeks old, body weight range at study start was 245.8-317 g.

Experimental Design and Execution: In brief, after the animals were transferred to study, all animals were randomly assigned to treatment groups per the study design below. Animals were treated either via intravenous (IV) administration of formulation R1, or via intratracheal (IT) administration for formulation R2 of the test article. Each group had four animals euthanized at 6 time points post administration for sample collection as outlined in Table 7.

TABLE 7
Experimental Design

| | Dose | | | Number | |
|---|---|---|---|---|---|
| Group | (mg/kg) | Treatment | Route | of Animals | Time points |
| 1 | 1 | Formulation | IV | 24 | 1-3 min, |
| | | R1: Imatinib | | | 5 min, |
| | | mesylate | | | 10 min, |
| 2 | 1 | Formulation | IT | 24 | 20 min, |
| | | R2: Imatinib | | | 30 min, |
| | | free base | | | 60 min |

Pharmacokinetics

Plasma and lung concentration vs. time data was analyzed using JMP version 15.2.0. Averages for each time point are reported for groups/time points that have a minimum of 2 animals. The following PK parameters were estimated using two compartment IV bolus dose model fit using the non-linear model fit platform in JMP version 15.2.0: maximum observed concentration (Cₘₐₓ), time at which Cₘₐₓ is observed (Tₘₐₓ) and area under the plasma concentration vs. time curve from zero through the last measured concentration (AUC_{60 min}). AUC_{60 min} was calculated from the modeled curves using the trapezoidal integration method. Results are shown in Table 8 below.

TABLE 8
PK parameter results from lung and plasma concentration vs time data

| | Route of | Sample | | | | |
|---|---|---|---|---|---|---|
| Treatment | Administration | Location | Cmax | | Tₘₐₓ (min) | AUC_{60 min} |
| Formulation R1: | IV | Lung | 4920 | ng/g | 5 | 153231 |
| Imatinib mesylate | | Plasma | 173 | ng/mL | 5 | 16913 |
| Formulation R2: | IT | Lung | 17925 | ng/g | 2 | 239806 |
| Imatinib free base | | Plasma | 808 | ng/mL | 2 | 6890 |
| Therapeutic | | | | | | 4 |
| Advantage (Rd) | | | | | | |

The Therapeutic Advantage (Rd) in Table 8 was calculated using the following equation:

$$R d = \frac{{( {AUC}_{lung} / {AUC}_{p l a s m a} )}_{IT}}{{( {AUC}_{lung} / {AUC}_{p l a s m a} )}_{IV}}$$

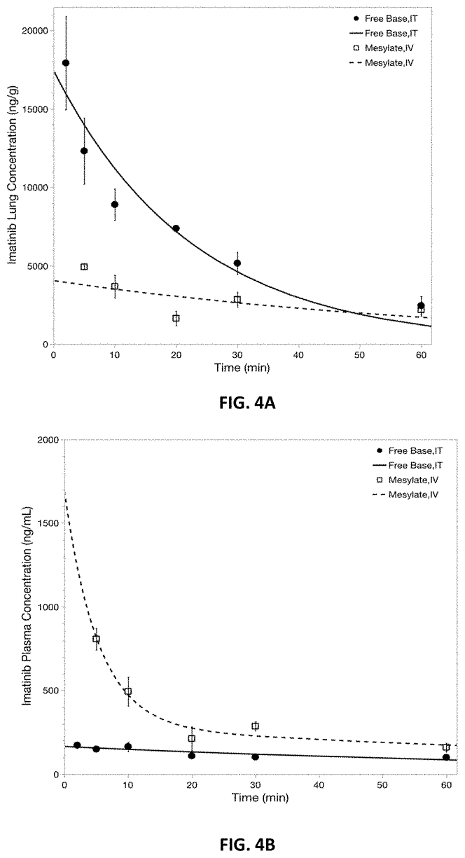

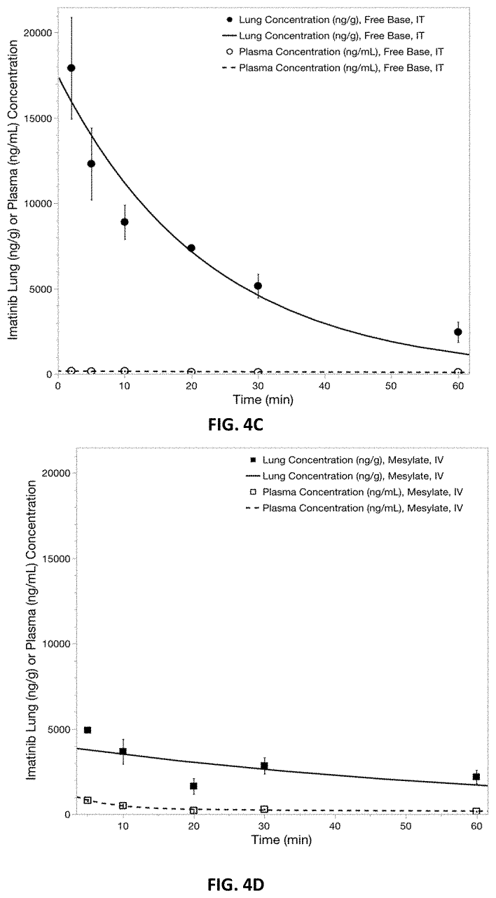

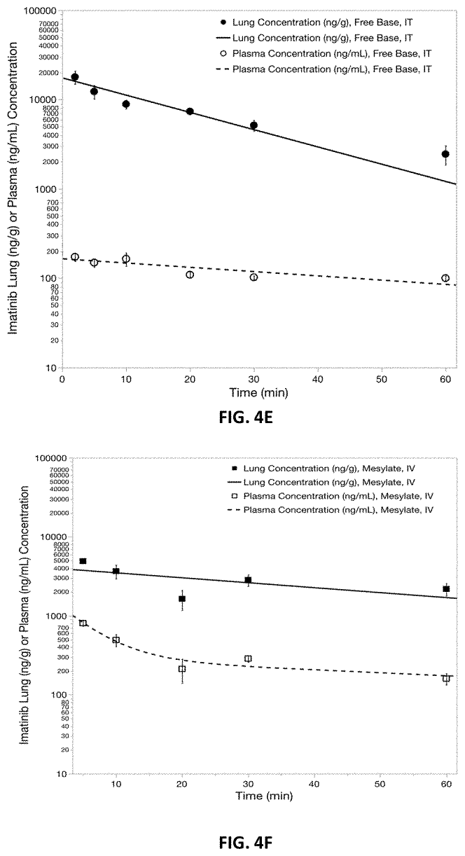

The IV data suggest that based on the high Log P (lipophilicity) of imatinib, the observed volume of distribution of imatinib (calculated as a ratio of amount of imatinib in the body versus plasma concentration of imatinib), and the cardiac output to the lungs, the sample collection period presented the distribution phase of the material preferentially accumulating in the lungs. This observation indicates direct administration of imatinib to the lungs can provide improved safety compared to systemic delivery via oral or IV routes. Since imatinib preferentially accumulates in the lung tissue direct delivery to the lungs via inhalation can minimize systemic exposure. Notably, direct delivery of imatinib to the lungs via IT administration of the imatinib free base/cyclodextrin formulation presents a 4 fold therapeutic advantage compared to IV administration of imatinib mesylate formulation (Rd=4). This therapeutic advantage can also be seen in FIGS. 4A-4F, which show plots summarizing concentration of imatinib in lung versus plasma over time after IT administration of imatinib free base/cyclodextrin formulation or IV administration of imatinib mesylate formulation.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A composition, comprising an aqueous solution or suspension that comprises imatinib or a derivative thereof, and a cyclodextrin or a pharmaceutically acceptable salt thereof, wherein said aqueous solution or suspension has said cyclodextrin at a concentration of from about 1% (w/v) to about 80% (w/v).

2. The composition of claim 1, wherein said cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, a sulfoalkylether-β-cyclodextrin, and a sulfoalkylether-γ-cyclodextrin.

3. The composition of claim 1, wherein said cyclodextrin is an anionic cyclodextrin.

4. The composition of claim 1, wherein said aqueous solution or suspension comprises a pharmaceutically acceptable salt of said cyclodextrin.

5. The composition of claim 1, wherein said cyclodextrin is sulfobutylether-β-cyclodextrin.

6. The composition of claim 1, wherein said aqueous solution or suspension comprises sulfobutylether-β-cyclodextrin sodium.

7. The composition of claim 1, wherein said aqueous solution or suspension further comprises a pH buffer.

8. The composition of claim 7, wherein said pH buffer is an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

9. The composition of claim 1, wherein said aqueous solution or suspension further comprises a surfactant.

10. The composition of claim 9, wherein said surfactant is Tween, sodium lauryl sulfate (SLS), or dipalmitoylphosphatidylcholine (DPPC).

11. The composition of claim 1, wherein said aqueous solution or suspension has a viscosity of at most 10 centipoise.

12. The composition of claim 1, wherein said aqueous solution or suspension has from 60 mg/mL to 120 mg/mL of said imatinib or derivative thereof.

13. The composition of claim 1, wherein said aqueous solution or suspension has about 80 mg/mL of said imatinib or derivative thereof.

14. The composition of claim 1, wherein said aqueous solution or suspension has a pH of 4 to 6.

15. The composition of claim 14, wherein said pH of said aqueous solution or suspension is about 5.0.

16. The composition of claim 1, wherein said aqueous solution or suspension has said cyclodextrin at a concentration of from 10% (w/v) to 20% (w/v).

17. The composition of claim 1, wherein said aqueous solution or suspension has said cyclodextrin at a concentration of from 25% (w/v) to 40% (w/v).

18. The composition of claim 1, wherein said composition comprises said aqueous solution.

19. The composition of claim 18, wherein said composition comprises less than 1 mg/mL imatinib mesylate.

20. The composition of claim 1, wherein said imatinib or derivative thereof is imatinib free base.

21. A pharmaceutical composition, comprising an aqueous solution that comprises cyclodextrin and a therapeutically effective amount of imatinib or a derivative thereof, wherein said aqueous solution is formulated for inhalatory administration.

22. A method of treating a subject having a pulmonary disease, comprising administering to said subject in need thereof via inhalation a pharmaceutical composition, wherein said pharmaceutical composition comprises an aqueous solution that comprises cyclodextrin and a therapeutically effective amount of imatinib or a derivative thereof.
